SEQUENCE LISTING (124 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 3 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - DNA, 3789 bp, synthetic construct
  misc_feature 1..3789: note = Synthetic construct
  source 1..3789: mol_type = other DNA
atgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtggagcacagatcatgatcggagcagcagccctcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggtagtagccagagctcccaatcgtcttacgggcagcagtcctcctatcctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatgggcggaagtgaccgtggtggcttcaataaatttggtggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaa

SEQ ID NO: 5 - protein, 1262 aa, synthetic construct
  REGION 1..1262: note = Synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAALMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 6 - DNA, 1770 bp, synthetic construct
  misc_feature 1..1770: note = Synthetic construct
  source 1..1770: mol_type = other DNA
atgggtaagcctatccctaaccctctcctcggtctcgattcaacaagtttgatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgggcagttcaggatcatccgggcacactctctacgccccaggcgggtacgatattatgggctggctgatccagatcatgaacaggcccaatccccaggtcgagctgggacccgtggatacttcatgtgcactggtgctgtgcgacctgaagcagaaggatacccccgtggtgtacgcatccgaagcgttcctctacatgaccgggtactcgaacgcggaggtgctgggtcgcaactgccggttcctgcagagccccgacggaatggtcaagcccaagtccactcggaaatacgtggattccaacaccatcaatactatgaggaaggctatcgataggaacgcggaagtccaagtggaggtggtgaacttcaagaagaatggccagagattcgtcaatttccttaccatgatccctgtgcgcgatgagactggagagtaccgatactcaatgggcttccagtgtgaaaccgagtgttaa

SEQ ID NO: 7 - protein, 589 aa, synthetic construct
  REGION 1..589: note = Synthetic construct
MGKPIPNPLLGLDSTSLMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMGSSGSSGHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEC

SEQ ID NO: 8 - DNA, 1824 bp, synthetic construct
  misc_feature 1..1824: note = Synthetic construct
  source 1..1824: mol_type = other DNA
atgcacactctctacgccccaggcgggtacgatattatgggctggctgatccagatcatgaacaggcccaatccccaggtcgagctgggacccgtggatacttcatgtgcactggtgctgtgtgaccttaagcagaaggacacccctgtggtgtacgcctccgaagcattcctgtacatgaccgggtactcgaacgccgaagtgctgggacggaactgccgcttcctgcaatccccggatggaatggtgaagcctaagtcaacccgcaaatacgtggactccaacactatcaacaccatgcgcaaggccattgaccgcaatgctgaggtgcaagtggaagtggtgaacttcaagaagaacggacagcgcttcgtcaacttcctgactatgattcccgtgcgggacgaaaccggcgaataccggtacagcatggggtttcagtgcgagactgagggctccggcggcagcggtggctccggaggcagcggaggtaccggcggcagtggagggtctggtggcagcggaatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgcttggtaccatgggtaagcctatccctaaccctctcctcggtctcgattcaacataa

SEQ ID NO: 9 - protein, 607 aa, synthetic construct
  REGION 1..607: note = Synthetic construct
MHTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGSGGSGGSGGSGGTGGSGGSGGSGMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLGTMGKPIPNPLLGLDST

SEQ ID NO: 10 - RNA, 24 nt, synthetic construct
  misc_feature 1..24: note = Synthetic construct
  source 1..24: mol_type = other RNA
tgtgtgtgtgtgtgtgtgtgtgtg

SEQ ID NO: 11 - DNA, 112 bp, synthetic construct
  misc_feature 1..112: note = Synthetic construct
  source 1..112: mol_type = other DNA
cmgmcmgmcmgmcmgmcmgmcmgmcmgmcmgmtmgmtmgmtmgmtmgmtmgmtmgmtmgmtmgmtmgmtmgmtmgmtmgmcmgmcmgmcmgmcmgmcmgmcmgmcmgmcmgm

SEQ ID NO: 12 - RNA, 56 nt, synthetic construct
  misc_feature 1..56: note = Synthetic construct
  source 1..56: mol_type = other RNA
cgcgcgcgcgcgcgcgtgtgtgtgtgtgtgtgtgtgtgtgcgcgcgcgcgcgcgcg

SEQ ID NO: 13 - RNA, 68 nt, synthetic construct
  misc_feature 1..68: note = Synthetic construct
  source 1..68: mol_type = other RNA
gmamgmamgmamgmcmgmcmgmtmgmcmamgmamgmamcmtmtmgmgmtmgmgmtmgmcmamtmamam

SEQ ID NO: 14 - RNA, 34 nt, synthetic construct
  misc_feature 1..34: note = Synthetic construct
  source 1..34: mol_type = other RNA
gagagagcgcgtgtgtgtgtgtggtggtgcataa

SEQ ID NO: 15 - DNA, 29 bp, unidentified
  misc_feature 1..29: note = natural sequence
  source 1..29: mol_type = other DNA
gttcttttgttttgttttttaacacttgt

SEQ ID NO: 16 - DNA, 33 bp, unidentified
  misc_feature 1..33: note = natural sequence
  source 1..33: mol_type = other DNA
ttaagtgagtttttgttcttagataacccacat

SEQ ID NO: 17 - DNA, 25 bp, unidentified
  misc_feature 1..25: note = natural sequence
  source 1..25: mol_type = other DNA
ttgtggtgtgctttgcaggaggact

SEQ ID NO: 18 - DNA, 37 bp, unidentified
  misc_feature 1..37: note = natural sequence
  source 1..37: mol_type = other DNA
ttaaataaaactgatttaaataatatgtgtctttgtt

SEQ ID NO: 19 - RNA, 29 nt, unidentified
  misc_feature 1..29: note = natural sequence
  source 1..29: mol_type = other RNA
gttcttttgttttgttttttaacacttgt

SEQ ID NO: 20 - RNA, 33 nt, unidentified
  misc_feature 1..33: note = natural sequence
  source 1..33: mol_type = other RNA
ttaagtgagtttttgttcttagataacccacat

SEQ ID NO: 21 - RNA, 25 nt, unidentified
  misc_feature 1..25: note = natural sequence
  source 1..25: mol_type = other RNA
ttgtggtgtgctttgcaggaggact

SEQ ID NO: 22 - RNA, 37 nt, unidentified
  misc_feature 1..37: note = natural sequence
  source 1..37: mol_type = other RNA
ttaaataaaactgatttaaataatatgtgtctttgtt

SEQ ID NO: 23 - DNA, 286 bp, unidentified
  misc_feature 1..286: note = natural sequence
  source 1..286: mol_type = other DNA
acccccatttccttaattatttcaggaataattctcccgaaatcaggtcaaccccatcaccaaccaaccaaccagttgagttccagattctatgtgaattaaaaagtcaatatatgtataattctgagatgacttaggttggacattcaatgttgtgctatgaatttcctctttatgcagagtatctgtttgcttgcagagtggctttctggcttgctgccagcctgtgcatggaccacgcttatgagttcaggatctacggcaatgtgaatcattcagatgttta

SEQ ID NO: 24 - DNA, 35 bp, unidentified
  misc_feature 1..35: note = natural sequence
  source 1..35: mol_type = other DNA
ttggacattcaatgttgtgctatgaatttcctctt

SEQ ID NO: 25 - RNA, 286 nt, unidentified
  misc_feature 1..286: note = natural sequence
  source 1..286: mol_type = other RNA
acccccatttccttaattatttcaggaataattctcccgaaatcaggtcaaccccatcaccaaccaaccaaccagttgagttccagattctatgtgaattaaaaagtcaatatatgtataattctgagatgacttaggttggacattcaatgttgtgctatgaatttcctctttatgcagagtatctgtttgcttgcagagtggctttctggcttgctgccagcctgtgcatggaccacgcttatgagttcaggatctacggcaatgtgaatcattcagatgttta

SEQ ID NO: 26 - RNA, 35 nt, unidentified
  misc_feature 1..35: note = natural sequence
  source 1..35: mol_type = other RNA
ttggacattcaatgttgtgctatgaatttcctctt

SEQ ID NO: 27 - DNA, 23 bp, unidentified
  misc_feature 1..23: note = natural sequence
  source 1..23: mol_type = other DNA
gattaaaagttgtgttccccaat

SEQ ID NO: 28 - RNA, 23 nt, unidentified
  misc_feature 1..23: note = natural sequence
  source 1..23: mol_type = other RNA
gattaaaagttgtgttccccaat

SEQ ID NO: 29 - DNA, 46 bp, unidentified
  misc_feature 1..46: note = natural sequence
  source 1..46: mol_type = other DNA
gagacctggttgtgtgtgtgtgagtggttgaccttcctccatcccc

SEQ ID NO: 30 - RNA, 46 nt, unidentified
  misc_feature 1..46: note = natural sequence
  source 1..46: mol_type = other RNA
gagacctggttgtgtgtgtgtgagtggttgaccttcctccatcccc

SEQ ID NO: 31 - DNA, 28 bp, unidentified
  misc_feature 1..28: note = natural sequence
  source 1..28: mol_type = other DNA
gtgtttgtgtgtgtgcgcgtgtgcgttc

SEQ ID NO: 32 - RNA, 28 nt, unidentified
  misc_feature 1..28: note = natural sequence
  source 1..28: mol_type = other RNA
gtgtttgtgtgtgtgcgcgtgtgcgttc

SEQ ID NO: 33 - DNA, 12 bp, synthetic construct
  misc_feature 1..12: note = Synthetic construct
  misc_feature 1..12: note = repeat_unit -
  source 1..12: mol_type = other DNA
atgtgtgtgtgt

SEQ ID NO: 34 - DNA, 12 bp, synthetic construct
  misc_feature 1..12: note = Synthetic construct
  misc_feature 1..12: note = repeat_unit -
  source 1..12: mol_type = other DNA
gtatgtgtgtgt

SEQ ID NO: 35 - DNA, 12 bp, synthetic construct
  misc_feature 1..12: note = Synthetic construct
  misc_feature 1..12: note = repeat_unit -
  source 1..12: mol_type = other DNA
gtgtatgtgtgt

SEQ ID NO: 36 - DNA, 12 bp, synthetic construct
  misc_feature 1..12: note = Synthetic construct
  misc_feature 1..12: note = repeat_unit -
  source 1..12: mol_type = other DNA
gtgtgtatgtgt

SEQ ID NO: 37 - DNA, 12 bp, synthetic construct
  misc_feature 1..12: note = Synthetic construct
  misc_feature 1..12: note = repeat_unit -
  source 1..12: mol_type = other DNA
gtgtgtgtgtat

SEQ ID NO: 38 - DNA, 12 bp, synthetic construct
  misc_feature 1..12: note = Synthetic construct
  misc_feature 1..12: note = repeat_unit -
  source 1..12: mol_type = other DNA
tatgtgtgtgtg

SEQ ID NO: 39 - DNA, 12 bp, synthetic construct
  misc_feature 1..12: note = Synthetic construct
  misc_feature 1..12: note = repeat_unit -
  source 1..12: mol_type = other DNA
tgtatgtgtgtg

SEQ ID NO: 40 - DNA, 12 bp, synthetic construct
  misc_feature 1..12: note = Synthetic construct
  misc_feature 1..12: note = repeat_unit -
  source 1..12: mol_type = other DNA
tgtgtatgtgtg

SEQ ID NO: 41 - DNA, 12 bp, synthetic construct
  misc_feature 1..12: note = Synthetic construct
  misc_feature 1..12: note = repeat_unit -
  source 1..12: mol_type = other DNA
tgtgtatgtgtg

SEQ ID NO: 42 - DNA, 12 bp, synthetic construct
  misc_feature 1..12: note = Synthetic construct
  misc_feature 1..12: note = repeat_unit -
  source 1..12: mol_type = other DNA
tgtgtgtatgtg

SEQ ID NO: 43 - DNA, 12 bp, synthetic construct
  misc_feature 1..12: note = Synthetic construct
  misc_feature 1..12: note = repeat_unit -
  source 1..12: mol_type = other DNA
tgtgtgtgtatg

SEQ ID NO: 44 - DNA, 12 bp, synthetic construct
  misc_feature 1..12: note = Synthetic construct
  misc_feature 1..12: note = repeat_unit -
  source 1..12: mol_type = other DNA
tgtgtgtgtgta

SEQ ID NO: 45 - RNA, 12 nt, synthetic construct
  misc_feature 1..12: note = Synthetic construct
  misc_feature 1..12: note = repeat_unit -
  source 1..12: mol_type = other RNA
atgtgtgtgtgt

SEQ ID NO: 46 - RNA, 12 nt, synthetic construct
  misc_feature 1..12: note = Synthetic construct
  misc_feature 1..12: note = repeat_unit -
  source 1..12: mol_type = other RNA
gtatgtgtgtgt

SEQ ID NO: 47 - RNA, 12 nt, synthetic construct
  misc_feature 1..12: note = Synthetic construct
  misc_feature 1..12: note = repeat_unit -
  source 1..12: mol_type = other RNA
gtgtatgtgtgt

SEQ ID NO: 48 - RNA, 12 nt, synthetic construct
  misc_feature 1..12: note = Synthetic construct
  misc_feature 1..12: note = repeat_unit -
  source 1..12: mol_type = other RNA
gtgtgtatgtgt

SEQ ID NO: 49 - RNA, 12 nt, synthetic construct
  misc_feature 1..12: note = Synthetic construct
  misc_feature 1..12: note = repeat_unit -
  source 1..12: mol_type = other RNA
gtgtgtgtgtat

SEQ ID NO: 50 - RNA, 12 nt, synthetic construct
  misc_feature 1..12: note = Synthetic construct
  misc_feature 1..12: note = repeat_unit -
  source 1..12: mol_type = other RNA
tatgtgtgtgtg

SEQ ID NO: 51 - RNA, 12 nt, synthetic construct
  misc_feature 1..12: note = Synthetic construct
  misc_feature 1..12: note = repeat_unit -
  source 1..12: mol_type = other RNA
tgtatgtgtgtg

SEQ ID NO: 52 - RNA, 12 nt, synthetic construct
  misc_feature 1..12: note = Synthetic construct
  misc_feature 1..12: note = repeat_unit -
  source 1..12: mol_type = other RNA
tgtgtatgtgtg

SEQ ID NO: 53 - RNA, 12 nt, synthetic construct
  misc_feature 1..12: note = Synthetic construct
  misc_feature 1..12: note = repeat_unit -
  source 1..12: mol_type = other RNA
tgtgtatgtgtg

SEQ ID NO: 54 - RNA, 12 nt, synthetic construct
  misc_feature 1..12: note = Synthetic construct
  misc_feature 1..12: note = repeat_unit -
  source 1..12: mol_type = other RNA
tgtgtgtatgtg

SEQ ID NO: 55 - RNA, 12 nt, synthetic construct
  misc_feature 1..12: note = Synthetic construct
  misc_feature 1..12: note = repeat_unit -
  source 1..12: mol_type = other RNA
tgtgtgtgtatg

SEQ ID NO: 56 - RNA, 12 nt, synthetic construct
  misc_feature 1..12: note = Synthetic construct
  misc_feature 1..12: note = repeat_unit -
  source 1..12: mol_type = other RNA
tgtgtgtgtgta

SEQ ID NO: 57 - DNA, 70 bp, unidentified
  misc_feature 1..70: note = natural sequence
  source 1..70: mol_type = other DNA
ggtccagaagcgtgtgtgtgtatgtgtgtgtgtgtgtgtgtgtgtgtgtgtgtgtgtgtgtgtttgtgtg

SEQ ID NO: 58 - RNA, 70 nt, unidentified
  misc_feature 1..70: note = natural sequence
  source 1..70: mol_type = other RNA
ggtccagaagcgtgtgtgtgtatgtgtgtgtgtgtgtgtgtgtgtgtgtgtgtgtgtgtgtgtttgtgtg

SEQ ID NO: 59 - RNA, 34 nt, unidentified
  misc_feature 1..34: note = natural sequence
  source 1..34: mol_type = other RNA
gagagagcgcgtgcagagacttggtggtgcataa

SEQ ID NO: 60 - RNA, 34 nt, unidentified
  misc_feature 1..34: note = natural sequence
  source 1..34: mol_type = other RNA
taggagggtcagaactgagagtggtacatcgcgg

SEQ ID NO: 61 - DNA, 56 bp, synthetic construct
  misc_feature 1..56: note = Synthetic construct
  source 1..56: mol_type = other DNA
cgcgcgcgcgcgcgcgtgtgtgtgtgtgtgtgtgtgtgtgcgcgcgcgcgcgcgcg

SEQ ID NO: 62 - DNA, 20 bp, synthetic construct
  misc_feature 1..20: note = Synthetic construct
  source 1..20: mol_type = other DNA
tgaatgtgtgtgtgtgttgt

SEQ ID NO: 63 - RNA, 20 nt, synthetic construct
  misc_feature 1..20: note = Synthetic construct
  source 1..20: mol_type = other RNA
tgaatgtgtgtgtgtgttgt

SEQ ID NO: 64 - RNA, 36 nt, synthetic construct
  misc_feature 1..36: note = Synthetic construct
  source 1..36: mol_type = other RNA
tgtgtgtgtgtgtgtgtgtgtgtgtgtgtgtgtgtg

SEQ ID NO: 65 - RNA, 32 nt, synthetic construct
  misc_feature 1..32: note = Synthetic construct
  source 1..32: mol_type = other RNA
tgtgtgtgtgtgtgtgtgtgtgtgtgtgtgtg

SEQ ID NO: 66 - RNA, 28 nt, synthetic construct
  misc_feature 1..28: note = Synthetic construct
  source 1..28: mol_type = other RNA
tgtgtgtgtgtgtgtgtgtgtgtgtgtg

SEQ ID NO: 67 - RNA, 22 nt, synthetic construct
  misc_feature 1..22: note = Synthetic construct
  source 1..22: mol_type = other RNA
tgtgtgtgtgtgtgtgtgtgtg

SEQ ID NO: 68 - RNA, 20 nt, synthetic construct
  misc_feature 1..20: note = Synthetic construct
  source 1..20: mol_type = other RNA
tgtgtgtgtgtgtgtgtgtg

SEQ ID NO: 69 - RNA, 18 nt, synthetic construct
  misc_feature 1..18: note = Synthetic construct
  source 1..18: mol_type = other RNA
tgtgtgtgtgtgtgtgtg

SEQ ID NO: 70 - RNA, 16 nt, synthetic construct
  misc_feature 1..16: note = Synthetic construct
  source 1..16: mol_type = other RNA
tgtgtgtgtgtgtgtg

SEQ ID NO: 71 - RNA, 14 nt, synthetic construct
  misc_feature 1..14: note = Synthetic construct
  source 1..14: mol_type = other RNA
tgtgtgtgtgtgtg

SEQ ID NO: 72 - RNA, 12 nt, synthetic construct
  misc_feature 1..12: note = Synthetic construct
  source 1..12: mol_type = other RNA
tgtgtgtgtgtg

SEQ ID NO: 73 - RNA, 10 nt, synthetic construct
  misc_feature 1..10: note = Synthetic construct
  source 1..10: mol_type = other RNA
tgtgtgtgtg

SEQ ID NO: 74 - DNA, 24 bp, synthetic construct
  misc_feature 1..24: note = Synthetic construct
  source 1..24: mol_type = other DNA
tgtgtgtgtgtgtgtgtgtgtgtg

SEQ ID NO: 75 - DNA, 36 bp, synthetic construct
  misc_feature 1..36: note = Synthetic construct
  source 1..36: mol_type = other DNA
tgtgtgtgtgtgtgtgtgtgtgtgtgtgtgtgtgtg

SEQ ID NO: 76 - DNA, 32 bp, synthetic construct
  misc_feature 1..32: note = Synthetic construct
  source 1..32: mol_type = other DNA
tgtgtgtgtgtgtgtgtgtgtgtgtgtgtgtg

SEQ ID NO: 77 - DNA, 28 bp, synthetic construct
  misc_feature 1..28: note = Synthetic construct
  source 1..28: mol_type = other DNA
tgtgtgtgtgtgtgtgtgtgtgtgtgtg

SEQ ID NO: 78 - DNA, 22 bp, synthetic construct
  misc_feature 1..22: note = Synthetic construct
  source 1..22: mol_type = other DNA
tgtgtgtgtgtgtgtgtgtgtg

SEQ ID NO: 79 - DNA, 20 bp, synthetic construct
  misc_feature 1..20: note = Synthetic construct
  source 1..20: mol_type = other DNA
tgtgtgtgtgtgtgtgtgtg

SEQ ID NO: 80 - DNA, 18 bp, synthetic construct
  misc_feature 1..18: note = Synthetic construct
  source 1..18: mol_type = other DNA
tgtgtgtgtgtgtgtgtg

SEQ ID NO: 81 - DNA, 16 bp, synthetic construct
  misc_feature 1..16: note = Synthetic construct
  source 1..16: mol_type = other DNA
tgtgtgtgtgtgtgtg

SEQ ID NO: 82 - DNA, 14 bp, synthetic construct
  misc_feature 1..14: note = Synthetic construct
  source 1..14: mol_type = other DNA
tgtgtgtgtgtgtg

SEQ ID NO: 83 - DNA, 12 bp, synthetic construct
  misc_feature 1..12: note = Synthetic construct
  source 1..12: mol_type = other DNA
tgtgtgtgtgtg

SEQ ID NO: 84 - DNA, 10 bp, synthetic construct
  misc_feature 1..10: note = Synthetic construct
  source 1..10: mol_type = other DNA
tgtgtgtgtg

SEQ ID NO: 85 - DNA, 41 bp, synthetic construct
  misc_feature 1..41: note = Synthetic construct
  source 1..41: mol_type = other DNA
gtgtgtgtgtgtgtgtgtgtgtgtgtgtgtgtgtgtgtatg

SEQ ID NO: 86 - DNA, 21 bp, synthetic construct
  misc_feature 1..21: note = Synthetic construct
  source 1..21: mol_type = other DNA
gtggttgtgtgtgtgtggtat

SEQ ID NO: 87 - RNA, 41 nt, synthetic construct
  misc_feature 1..41: note = Synthetic construct
  source 1..41: mol_type = other RNA
gtgtgtgtgtgtgtgtgtgtgtgtgtgtgtgtgtgtgtatg

SEQ ID NO: 88 - RNA, 21 nt, synthetic construct
  misc_feature 1..21: note = Synthetic construct
  source 1..21: mol_type = other RNA
gtggttgtgtgtgtgtggtat

SEQ ID NO: 89 - DNA, 28 bp, synthetic construct
  misc_feature 1..28: note = Synthetic construct
  source 1..28: mol_type = other DNA
cgcgcgcgcgcgcgcgcgcgcgcgcgcg

SEQ ID NO: 90 - DNA, 24 bp, synthetic construct
  misc_feature 1..24: note = Synthetic construct
  source 1..24: mol_type = other DNA
cgcgcgcgcgcgcgcgcgcgcgcg

SEQ ID NO: 91 - DNA, 20 bp, synthetic construct
  misc_feature 1..20: note = Synthetic construct
  source 1..20: mol_type = other DNA
cgcgcgcgcgcgcgcgcgcg

SEQ ID NO: 92 - DNA, 16 bp, synthetic construct
  misc_feature 1..16: note = Synthetic construct
  source 1..16: mol_type = other DNA
cgcgcgcgcgcgcgcg

SEQ ID NO: 93 - DNA, 14 bp, synthetic construct
  misc_feature 1..14: note = Synthetic construct
  source 1..14: mol_type = other DNA
cgcgcgcgcgcgcg

SEQ ID NO: 94 - DNA, 12 bp, synthetic construct
  misc_feature 1..12: note = Synthetic construct
  source 1..12: mol_type = other DNA
cgcgcgcgcgcg

SEQ ID NO: 95 - DNA, 10 bp, synthetic construct
  misc_feature 1..10: note = Synthetic construct
  source 1..10: mol_type = other DNA
cgcgcgcgcg

SEQ ID NO: 96 - RNA, 48 nt, unidentified
  misc_feature 1..48: note = natural sequence
  source 1..48: mol_type = other RNA
gaggtggctatggaggtggctatggaggtggctatggaggtggctatg

SEQ ID NO: 97 - RNA, 24 nt, unidentified
  misc_feature 1..24: note = natural sequence
  source 1..24: mol_type = other RNA
gactgaaaaaggtgggtttctttt

SEQ ID NO: 98 - RNA, 25 nt, unidentified
  misc_feature 1..25: note = natural sequence
  source 1..25: mol_type = other RNA
ctaggatggaggtggggaatggtac

SEQ ID NO: 99 - RNA, 48 nt, synthetic construct
  misc_feature 1..48: note = Synthetic construct
  source 1..48: mol_type = other RNA
attgaggagcagcagagaagttggagtgaaggcagagaggggttaagg

SEQ ID NO: 100 - RNA, 48 nt, synthetic construct
  misc_feature 1..48: note = Synthetic construct
  source 1..48: mol_type = other RNA
attgaggagcagcagagaagttggaaaaaaaaaaaaaaaaaaaaaaaa

SEQ ID NO: 101 - RNA, 48 nt, synthetic construct
  misc_feature 1..48: note = Synthetic construct
  source 1..48: mol_type = other RNA
aaaaaaaaaaaaaaaaaaaaaaaaagtgaaggcagagaggggttaagg

SEQ ID NO: 102 - RNA, 39 nt, unidentified
  misc_feature 1..39: note = natural sequence
  source 1..39: mol_type = other RNA
accatgatcacgaaggtggttttcccagggcgaggctta

SEQ ID NO: 103 - RNA, 43 nt, unidentified
  misc_feature 1..43: note = natural sequence
  source 1..43: mol_type = other RNA
ctccggatgtgctgacccctgcgatttccccaaatgtgggaaa

SEQ ID NO: 104 - RNA, 28 nt, unidentified
  misc_feature 1..28: note = natural sequence
  source 1..28: mol_type = other RNA
gaaaattaatgtgtgtgtgtggaaaatt

SEQ ID NO: 105 - RNA, 25 nt, unidentified
  misc_feature 1..25: note = natural sequence
  source 1..25: mol_type = other RNA
ttgtattttgagctagtttggtgat

SEQ ID NO: 106 - RNA, 24 nt, synthetic construct
  misc_feature 1..24: note = Synthetic construct
  source 1..24: mol_type = other RNA
tcagagacatcatcagagacatca

SEQ ID NO: 107 - RNA, 24 nt, synthetic construct
  misc_feature 1..24: note = Synthetic construct
  source 1..24: mol_type = other RNA
ggtgagcacagaggtgagcacaga

SEQ ID NO: 108 - RNA, 24 nt, synthetic construct
  misc_feature 1..24: note = Synthetic construct
  source 1..24: mol_type = other RNA
ccaatcttccttccaatcttcctt

SEQ ID NO: 109 - RNA, 24 nt, synthetic construct
  misc_feature 1..24: note = Synthetic construct
  source 1..24: mol_type = other RNA
gatggattccaggatggattccag

SEQ ID NO: 110 - RNA, 24 nt, synthetic construct
  misc_feature 1..24: note = Synthetic construct
  source 1..24: mol_type = other RNA
aaacggtctgataaacggtctgat

SEQ ID NO: 111 - RNA, 40 nt, synthetic construct
  misc_feature 1..40: note = Synthetic construct
  source 1..40: mol_type = other RNA
atgtcgcagaatgtcgcagaatgtcgcagaatgtcgcaga

SEQ ID NO: 112 - RNA, 40 nt, synthetic construct
  misc_feature 1..40: note = Synthetic construct
  source 1..40: mol_type = other RNA
aaccttcgtaaaccttcgtaaaccttcgtaaaccttcgta

SEQ ID NO: 113 - RNA, 24 nt, synthetic construct
  misc_feature 1..24: note = Synthetic construct
  source 1..24: mol_type = other RNA
cgctggcatccacgctggcatcca

SEQ ID NO: 114 - RNA, 24 nt, synthetic construct
  misc_feature 1..24: note = Synthetic construct
  source 1..24: mol_type = other RNA
aaagcggcgatgaaagcggcgatg

SEQ ID NO: 115 - RNA, 24 nt, synthetic construct
  misc_feature 1..24: note = Synthetic construct
  source 1..24: mol_type = other RNA
tattgatccggttattgatccggt

SEQ ID NO: 116 - RNA, 50 nt, synthetic construct
  misc_feature 1..50: note = Synthetic construct
  source 1..50: mol_type = other RNA
cmtmamgmgmamtmgmgmamgmgmtmgmgmgmgmamamtmgmgmtmamcm

SEQ ID NO: 117 - RNA, 24 nt, synthetic construct
  misc_feature 1..24: note = Synthetic construct
  source 1..24: mol_type = other RNA
gaggtggctatggaggtggctatg

SEQ ID NO: 118 - RNA, 24 nt, synthetic construct
  misc_feature 1..24: note = Synthetic construct
  source 1..24: mol_type = other RNA
gtggtggctatggaggtggctatg

SEQ ID NO: 119 - RNA, 48 nt, synthetic construct
  misc_feature 1..48: note = Synthetic construct
  source 1..48: mol_type = other RNA
tcagagacatcatcagagacatcatcagagacatcatcagagacatca

SEQ ID NO: 120 - DNA, 112 bp, synthetic construct
  misc_feature 1..112: note = Synthetic construct
  source 1..112: mol_type = other DNA
cmgmcmgmcmgmcmgmcmgmcmgmcmgmcmgmamcmamcmamcmamcmamcmamcmamcmamcmamcmamcmamcmamcmcmgmcmgmcmgmcmgmcmgmcmgmcmgmcmgm

SEQ ID NO: 121 - RNA, 24 nt, synthetic construct
  misc_feature 1..24: note = Synthetic construct
  source 1..24: mol_type = other RNA
tgtattttgagctagtttgctgat

SEQ ID NO: 122 - DNA, 27 bp, synthetic construct
  misc_feature 1..27: note = Probe
  variation 27: note = n is a, c, g, or t
  modified_base 27: mod_base = OTHER; note = nucleotide bound to label, Digoxigenin-label
  source 1..27: mol_type = other DNA
tttttttttttttttttttttttttvn

SEQ ID NO: 123 - DNA, 22 bp, synthetic construct
  misc_feature 1..22: note = Probe
  modified_base 1: mod_base = OTHER; note = nucleotide bound to label, Digoxigenin-label
  source 1..22: mol_type = other DNA
gtgtaacacgtctatacgccca

SEQ ID NO: 124 - protein, 14 aa, synthetic construct
  REGION 1..14: note = an amino acid sequence of viral RNA polymerase
GKPIPNPLLGLDST

(19) United States
(12) Patent Application Publication
(10) Pub. No.: US 2026/0263500 A1
(43) Pub. Date: Sep. 10, 2026

(54) NUCLEIC ACIDS AND NUCLEIC ACID ANALOGS FOR TREATING, PREVENTING, AND DISRUPTING PATHOLOGICAL POLYNUCLEOTIDE-BINDING PROTEIN INCLUSIONS

(71) Applicants: UNIVERSITY OF PITTSBURGH-OF THE COMMONWEALTH SYSTEM OF HIGHER EDUCATION, Pittsburgh, PA (US); THE TRUSTEES OF THE UNIVERSITY OF PENNSYLVANIA, Philadelphia, PA (US)

(72) Inventors: Christopher DONNELLY, Pittsburgh, PA (US); Jacob MANN, Pittsburgh, PA (US); James SHORTER, Philadelphia, PA (US); Lin GUO, Philadelphia, PA (US); Bede PORTZ, Philadelphia, PA (US)

(21) Appl. No.: 19/416,906

(22) Filed: Dec. 11, 2025

Related U.S. Application Data
(62) Division of application No. 17/268,654, filed on Aug. 16, 2019, now Pat. No. 12,521,412.
(60) Provisional application No. 62/764,828, filed on Aug. 16, 2018.

(51) Int. Cl. A61K 31/7088; A61P 25/28

(52) U.S. Cl. CPC A61K 31/7088; A61P 25/28

(57) ABSTRACT

Disclosed herein are methods to treat a neurodegenerative disease comprising administering to a subject with a neurodegenerative disease a therapeutically effective amount of a polynucleotide that binds a nucleic acid-binding polypeptide, wherein the nucleic acid-binding polypeptide can aggregate in cells and is associated with a neurodegenerative disease. Also disclosed are methods to inhibit protein aggregation in a cell comprising contacting the cell with a composition comprising a polynucleotide that binds a nucleic acid-binding polypeptide, wherein the nucleic acid-binding polypeptide can aggregate in cells and is associated with a neurodegenerative disease. Additionally disclosed are compositions comprising a polynucleotide that binds a nucleic acid-binding polypeptide, wherein the nucleic acid-binding polypeptide can aggregate in cells and is associated with a neurodegenerative disease.

### STATEMENT REGARDING FEDERALLY SPONSORED RESEARCH

This invention was made with government support under Grant No. NS105756 awarded by the National Institutes of Health. The government has certain rights in the invention.

### REFERENCE TO SEQUENCE LISTING

The Sequence Listing submitted May 18, 2026, as an .XML file titled “10504-029US2-ST26.xml”, created on Mar. 19, 2026, and having a size of 179,872 bytes is hereby incorporated by reference pursuant to 37 C.F.R. § 1.52(e)(5).

### FIELD

The disclosure generally relates to neurodegenerative diseases such as Amyotrophic Lateral Sclerosis, Frontotemporal Dementia, and Chronic Traumatic Encephalopathy, and methods and compositions comprising polynucleotides for treating neurodegenerative diseases.

### BACKGROUND

Amyotrophic Lateral Sclerosis (ALS) and Frontotemporal Dementia (FTD) are fatal neurodegenerative disorders characterized by the progressive loss of motor neurons of the spinal cord and motor cortex or cortical neurons of the frontal and temporal lobes, respectively. No effective treatments currently exist to halt ALS or FTD progression and the cause(s) of these disorders remain unknown. Significant overlap of clinical, genetic, and neuropathological features among patients suggests that ALS and FTD exist on a neurodegenerative disease spectrum (Ling, et al., (2013), Neuron 79, 416-438), and a number of familial ALS and/or FTD causing mutations have been identified (Nguyen, et al., (2018), Trends Genet. 34, 404-423). Despite this vast genetic heterogeneity, 97% of ALS patients and up to 45% of FTD patients exhibit a common neuropathological feature called TDP-43 proteinopathy. TDP-43 proteinopathy is characterized by the cytoplasmic deposition and nuclear clearance of the transactivation response element DNA-binding protein 43 kDa (TDP-43; TARDBP) (Neumann, et al., (2006), Science 314, 130-133).

TDP-43 is a ubiquitously-expressed, tightly-regulated, and predominantly nuclear DNA/RNA-binding protein that contains two RNA-Recognition Motifs (RRMs) and a C-terminal glycine-rich, low complexity domain (LCD) (Scotter, et al., (2015), Neurotherapeutics 12, 352-363). TDP-43 preferentially binds UG-rich RNA intronic sequences or 3′UTR stem loop structures and influences a variety of RNA processing events including alternative splicing, RNA trafficking, and RNA stability (Ling, et al., (2013), Neuron 79, 416-438). In ALS and FTD, the protein is found in cytoplasmic inclusions that are detergent-insoluble, hyperphosphorylated, p62-positive, and ubiquitinated (Scotter, et al., (2015), Neurotherapeutics 12, 352-363). Cytoplasmic inclusions also contain truncated TDP-43 species resulting from its N-terminal cleavage (Neumann, et al., (2007), J. Neuropathol.

Exp. Neurol. 66, 177-183). This neuropathological hallmark correlates well with regions of neurodegeneration in ALS/FTD patient tissue (Baloh, R. H. (2011), FEBS J. 278, 3539-3549) and is found in postmortem tissue from patients diagnosed with Alzheimer's Disease (60%) (Youmans, et al., (2012), Exp. Neurol. 237, 90-95), and Chronic Traumatic Encephalopathy (80%) (McKee, et al., (2010), J. Neuropathol. Exp. Neurol. 69, 918-929). Thus, TDP-43 proteinopathy might serve as a convergence point of pathogenesis despite the diverse upstream mechanisms responsible for disease etiology across several neurodegenerative disorders.

Over 50 ALS/FTD-causing missense mutations have been identified in the TARDBP gene (Harrison, et al., (2017), Biochem. J. 474, 1417-1438). While rare in the total patient population, these mutations occur more frequently in fALS patients (5%) as compared to patients with familial FTD (Ling, et al., (2013), Neuron 79, 416-438). The majority of known ALS/FTD-causing TARDBP mutations cluster within the TDP-43 LCD, although others have been identified within the RRMs (Harrison, et al., (2017), Biochem. J. 474, 1417-1438). The location and functional impact of these mutations likely reflects the importance of these regions in disease pathogenesis. LCDs are common in RNA-binding proteins (RBPs) and mediate protein and RNA interactions through a process termed liquid-liquid phase separation (LLPS). LLPS involves the condensation of molecules into liquid-like compartments and is driven by weak, transient interactions between LCD regions and other multivalent protein/nucleic acid interaction domains (Harrison, et al., (2017), Biochem. J. 474, 1417-1438). Following specific protein:protein, protein:RNA and/or RNA:RNA nucleating interactions, this de-mixing process allows for intracellular compartmentalization, as observed with membraneless organelles such as nucleoli, P-bodies, and stress granules (SGs) (Shin, et al., (2017), Science 357). Many of these biological condensates contain high local concentrations of LCD-containing proteins, which contribute to the properties of these structures through a combination of specific and non-specific heterotypic protein/nucleic acid interactions (Harrison, et al., (2017), Biochem. J. 474, 1417-1438; Shin, et al., (2017), Science 357). Interestingly, ALS-associated mutations in the TDP-43 LCD alter LLPS behavior and enhance aggregation of the protein (Conicella, et al., (2016), Structure 24, 1537-1549; Johnson, et al., (2009), J. Biol. Chem. 284, 20329-20339; Schmidt, et al., (2016), Cell Rep. 16, 1228-1236). Similar observations have been reported of other RBPs implicated in neurodegeneration, where disease-linked LCD mutations or aging of droplets promotes the maturation and fibrillization of initially reversible protein assemblies (Harrison, et al., (2017), Biochem. J. 474, 1417-1438). While the physical processes underlying droplet solidification is unknown, these findings suggest that aberrant phase transitions drive the formation of pathological inclusions of RNA-binding proteins observed in neurodegenerative disease.

The cellular pathway(s) that promote aberrant TDP-43 phase transitions remain unclear, but evidence suggests that altered SG homeostasis contributes to the seeding of pathological inclusions. SGs are membraneless organelles that assemble in the cytoplasm via LLPS during periods of cellular stress and may reversibly inhibit non-essential protein synthesis (Anderson, et al., (2008), Trends Biochem. Sci. 33, 141-150). In addition to mRNA, ribosomal subunits, and translation initiation factors, SGs sequester a number of RBPs mutated in fALS, including TDP-43 (Boeynaems, et al., (2016), Acta Neuropathol. 132, 159-173). The nucleation and phase separation of these various proteins and RNAs into liquid-like droplets is required for cellular compartmentalization of SGs and alterations of intermolecular dynamics may promote an irreversible gel-like state or fibrillization of prion-like protein components of these structures. The high local concentration of aggregate-prone proteins, like TDP-43, within SGs is thought to enhance protein self-interactions that subsequently mature into pathological inclusions (Harrison, et al., (2017), Biochem. J. 474, 1417-1438). Supporting the role of SGs in seeding TDP-43 proteinopathy, recent work has also revealed that antisense oligonucleotide-mediated depletion of SG components ameliorates neurotoxicity in a TDP-43 rodent model (Becker, et al., (2017), Nature 544, 367-371).

Modeling TDP-43 proteinopathy has proven challenging. Current cellular and animal models rely on enhanced expression of wildtype or rare mutant variants of TDP-43 to initiate pathological aggregation; however, this approach is unreliable and many models fail to develop inclusions that recapitulate ALS/FTD phenotypes (Philips, et al., (2015), Curr. Protoc. Pharmacol. 69, 5.67.1-21). Furthermore, the lack of control over intracellular TDP-43 interactions impedes the ability to test whether cytoplasmic phase separation precedes inclusion formation and whether this process, or these inclusions themselves, are neurotoxic.

The compositions and methods disclosed herein address these and other needs.

### SUMMARY

The disclosed subject matter relates to compositions and methods for treating neurodegenerative diseases. In one aspect, disclosed herein is a method to treat a neurodegenerative disease comprising administering to a subject with a neurodegenerative disease a therapeutically effective amount of a polynucleotide that binds a nucleic acid-binding polypeptide, wherein the nucleic acid-binding polypeptide can aggregate in cells and is associated with a neurodegenerative disease.

In some embodiments, the neurodegenerative disease is selected from Limbic-predominant Age-related TDP-43 Encephalopathy (LATE), Multisystem Proteinopathy, Amyotrophic Lateral Sclerosis, Frontotemporal Dementia, Alzheimer's Disease, Chronic Traumatic Encephalopathy, Traumatic Brain Injury, Cortical Basal Degeneration, and Huntington's Disease. In some embodiments, the polynucleotide is administered in a pharmaceutical composition comprising a pharmaceutically acceptable excipient. In some embodiments, the method reduces the rate of aggregation of the nucleic acid-binding polypeptide in the subject. In some embodiments, the method reduces the amount of aggregate of the nucleic acid-binding polypeptide in the subject. In some embodiments, the nucleic acid-binding polypeptide has an increased rate of aggregation when the concentration of free polynucleotides is reduced. In some embodiments, the nucleic acid-binding polypeptide is selected from TDP-43, FUS, HNRNPA1, HNRNPA2/B1, EWSR1, TIA-1, TTP, TAF15, MATR3, ATXN2, OPTN, Tau, HTT, and biologically active fragments and variants thereof. In some embodiments, the polynucleotide comprises a synthetic RNA. In some embodiments, the polynucleotide comprises at least 24 consecutive nucleotides comprising at least 60% uracil and guanine nucleotides. In some embodiments, the method further comprises administering one or more additional polynucleotides.

Also disclosed are methods to inhibit protein aggregation in a cell comprising contacting the cell with a composition comprising a polynucleotide that binds a nucleic acid-binding polypeptide, wherein the nucleic acid-binding polypeptide can aggregate in cells and is associated with a neurodegenerative disease.

In some embodiments, the method reduces the rate of aggregation of the nucleic acid-binding polypeptide in the cell compared to an untreated control. In some embodiments, the method reduces the amount of aggregate of the nucleic acid-binding polypeptide in the cell. In some embodiments, the cell is a neuronal cell.

Also disclosed are compositions comprising a polynucleotide that binds a nucleic acid-binding polypeptide, wherein the nucleic acid-binding polypeptide can aggregate in cells and is associated with a neurodegenerative disease.

In some embodiments, the nucleic acid-binding polypeptide is selected from TDP-43, FUS, HNRNPA1, HNRNPA2/B1, EWSR1, TIA-1, TTP, TAF15, MATR3, ATXN2, OPTN, Tau, HTT, and biologically active fragments and variants thereof. In some embodiments, the polynucleotide comprises at least 24 consecutive nucleotides comprising at least 60% uracil and guanine nucleotides. In some embodiments, the composition further comprises one or more additional polynucleotides.

Also disclosed are pharmaceutical compositions comprising a pharmaceutically acceptable excipient and a polynucleotide that binds a nucleic acid-binding polypeptide, wherein the nucleic acid-binding polypeptide can aggregate in cells and is associated with a neurodegenerative disease. Also disclosed are compositions comprising a polynucleotide having at least 70% identity with any one of SEQ ID NO:10-84. Also disclosed are compositions comprising a polynucleotide having at least 70% identity with any one of SEQ ID NO:96-119.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same element(s) throughout the figures.

FIG. 1(A-B) is a set of images and a graph showing optoTDP43 is a light-inducible model of TDP-43 proteinopathy. FIG. 1A shows images of HEK293 cells expressing optoTDP43 exposed to blue light stimulation (bottom row) (approximately 0.3 mW/cm², 465 nm) or darkness (top row). Images were obtained at the indicated time points (from left to right: 10 hrs, 12, hrs, 14 hrs, 24 hrs). Cell nuclei are circled. Arrows indicate optoTDP43 cytoplasmic inclusion formation with light treatment. FIG. 1B is a graph showing percentage of cells exhibiting optoTDP43 inclusions over time using automated longitudinal live-imaging. Solid line: optoTDP43 without light; dotted line: optoTDP43 with light; dashed line: TDP43 without light; dotted and dashed line: TDP43 with light. n=494-791 cells. Data shown are mean+/−S.E.M. ****, p<0.0001. Scale bars=10 μm.

FIG. 2 is a set of images showing optogenetic phase transitions of TDP-43 LCD drives inclusion formation. Images of repetitive light-induced phase transitions of WT and ALS-linked mutant (M337V, Q331K, A321V) optoLCD proteins during a cycling light stimulation protocol consisting of a single blue light pulse (1 sec, 1% laser power) every 10 min for 40 min. Pre-stim refers to images taken before any blue light exposure. Post-stim refers to images taken after the first 1 sec blue light pulse. C1, C2, C3, and C4 refer to Cycle 1, Cycle 2, Cycle 3, and Cycle 4, respectively, and are images taken 10 minutes after each 1 sec blue light pulse (1 sec pulse, 10 minute incubation in darkness, obtain image, repeat cycle). Images shown are the final images acquired per cycle prior to subsequent light stimulation. Scale bars=10 μm.

FIG. 3(A-D) is a set of graphs showing RNA-binding prevents light-induced phase separation and aggregation of TDP-43. FIG. 3(A-C) include data for HEK293 cells expressing photoreceptors. FIG. 3A shows granules observed per cell expressing the Cry2olig photoreceptor fused to the TDP-43 LCD (top solid line) or to the TDP-43 RNA-binding regions (RRMs) (bottom dotted line). FIG. 3B shows granules observed per cell expressing the Cry2olig photoreceptor fused to TDP-43 LCD and functional (WT) RRMs (top solid line) or fused to TDP-43 LCD and RNA-binding deficient (5FL) RRMs (bottom dotted line). FIG. 3C shows granules observed per cell expressing the Cry2olig photoreceptor fused to TDP-43 LCD and functional (WT) FUS RRM (top solid line) or fused to TDP-43 LCD and FUS RNA-binding deficient (4FL) RRM (bottom dotted line). Cells were exposed to acute light stimulation (1-8 sec, 10% laser power, 488 nm). n=33-59 (FIG. 3A), 24-36 (FIG. 3B) and 31-45 cells (FIG. 3C). Data shown are mean+/−S.E.M. FIG. 3D shows the percentage of optoTDP-43-expressing HEK293 cells having cytoplasmic inclusions after received mock (left) or RNA (right; 2.5 μg HEK293 total mRNA) treatment 4 hours into a 16 hour chronic blue light treatment. Data points correspond to individual fields of view. Data shown are mean+/−S.E.M. **, p<0.01.

FIG. 4(A-C) show TDP-43 LLPS and aggregation is inhibited by RNA-binding. FIG. 4A is a set of differential interference contrast (DIC) images of purified TDP43 WT or 5FL fused to maltose binding protein (MBP) following incubation with increasing concentrations (from left to right: 0 μg, 2.5 μg, 12.5 μg, 25 μg) of yeast total RNA. In the absence of RNA, C-terminal MBP-tagged TDP-43 WT and 5FL formed liquid-like droplets at physiological salt concentrations. However, in the presence of increasing concentrations of total RNA, WT TDP-43 (but not TDP-43 5FL) LLPS was inhibited in a dose-dependent manner. Scale bar=50 μm. FIG. 4B shows turbidity changes (normalized OD₃₉₅ readings) of TDP-43 WT (solid lines) and 5FL (dotted lines) proteins following TEV cleavage of MBP in the absence (open circles) or presence (solid circles) of yeast total RNA (15 μg). FIG. 4C shows turbidity changes (normalized OD₃₉₅ readings) of TDP-43 WT proteins following TEV cleavage of MBP in the presence of yeast total RNA-only (25 μg) (solid line) or yeast total RNA (25 μg) followed by RNase A addition (2.5 μg) at 90 min post-TEV cleavage (dotted line). Data shown are mean+/−S.E.M.

FIG. 5(A-D) is a set of graphs showing light-induced optoTDP43 phase transitions are neurotoxic. Lentiviral expression vectors were used in neuronal survival experiments. optoTDP43 or the Cry2-mCh photoreceptor alone were expressed along with a far-red fluorescent reporter (iRFP670) under the control of the human synapsin promoter (hSyn).

FIG. 5A shows survival curves of ReNcell cortical neurons during longitudinal toxicity screening in the presence or absence of light. Solid line: Cry2-mCH without light; dotted line: optoTDP43 without light; dashed line: Cry2-mCH with light; dotted and dashed line: optoTDP43 with light. n=74-89 cells. Data are presented as mean percent survival. FIG. 5B shows nuclear-cytoplasmic (N/C) ratios of optoTDP43 signal analyzed at baseline (prior to light exposure) and endpoints (last frame prior to cell death or conclusion of imaging session) in neurons exposed to light (open circles) or darkness (solid circles). n=25 cells per light condition. Data is presented as fold-changes in N/C ratios normalized to baseline values. Inset shows raw N/C values and data points represent individual neurons. Dotted lines indicate population mean at baseline. FIG. 5C shows survival curves of optoTDP43-expressing neurons exposed to light stimulation stratified by one of three optoTDP43 assembly phenotypes: inclusion (solid line), particles (dotted line), or diffuse nuclear phenotype (dashed line). n=13-28 cells. FIG. 5D shows time-to-death between event onset (particle or inclusion formation) and cell death analyzed between neurons showing either inclusion (solid line) or particle (dotted line) optoTDP43 assembly subtype. n=17-28 cells. Data shown are mean+/−S.E.M. ****, p<0.0001.

FIG. 6(A-D) is a set of graphs showing bait oligonucleotides (bONs) inhibit aberrant phase transitions of TDP-43 and rescue associated neurotoxicity. In FIG. 6A, HEK293 cells expressing optoTDP43 were pre-treated with non-targeting scrambled or targeting 2′OMe-modified Clip_34nt RNA oligonucleotides (SEQ ID NO:13) for 30 min prior to chronic blue light stimulation (8 hr, ~0.3 mW/cm², 465 nm). FIG. 6A shows quantification of percentage of cells having light-induced cytoplasmic optoTDP43 assemblies following the treatment with either scramble control RNA (left set of three bars in black) or Clip_34nt RNA (right set of three bars in gray) at the indicated concentrations (200 nM, 500 nM, or 1,000 nM). n=578 -943 cells. FIG. 6B-D show results for optoTDP43-expressing ReNcell neurons treated with non-targeting scrambled or targeting 2′OMe-modified Clip_34nt RNA oligonucleotides 4 hr prior to blue light exposure and automated longitudinal imaging. In FIG. 6B-6D, solid line: 500 nM Clip_34nt; dotted and dashed line: 1 μM Clip_34nt; dotted line: 500 nM scrambled; dashed line: 1 μM scrambled. FIG. 6B shows cumulative risk-of-death plots generated from Kaplan-Meier survival curves of ReNcell neurons over time following treatment with increasing doses of scrambled or targeting Clip_34nt oligonucleotides. n=78-121 cells. FIG. 6C shows percentage of ReNcell neurons having optoTDP43 assemblies (inclusions or particles) over time following the indicated oligonucleotide treatments. n=37-39 cells. FIG. 6D shows nuclear-cytoplasmic (N/C) ratios of optoTDP43 signal calculated over time in neurons exposed to the indicated oligonucleotide treatments. n=34-45 cells. *, p<0.05; **, p<0.01; ***, p<0.001; ****, p<0.0001. +indicate comparisons between 500 nM treatment groups; * indicate comparisons between 1000 nM treatment groups. Data shown are mean+/−S.E.M.

FIG. 7 is a graph showing poly UG RNA substrates (SEQ ID NO:10; solid line) prevent the formation of TDP-43 inclusions, as compared to mock RNA (dashed line).

FIG. 8 is a graph showing that the binding DNA oligonucleotide SL_TG24 (SEQ ID NO:11) by TDP-43 rescues TDP-43 aggregation toxicity in a dose dependent manner. Non-binding RNA AC24 was included as a control.

FIG. 9 is a graph showing quantification of normalized aggregation area observed in cells expressing VVD-TDP43-V5 inclusion formation after induction with 10 ng (left two bars) or 1,000 ng (right two bars) doxycycline and following exposure to darkness (light gray) or blue light (dark gray).

FIG. 10(A-B) is a set of graphs showing turbidity measurements each minute for 100 minutes when strong (FIG. 10A) and intermediate (FIG. 10B) RNA inhibitors were incubated with GST-TEV-FUS protein, TEV protease (to cleave the GST-TEV motif), and RNasin (to protect against RNA degradation). The tested samples in FIG. 10A included FUS alone (solid line), FUS+RNasin (dotted line), FUS+RNasin+RNA7 (SEQ ID NO:97), FUS+RNasin+RNA8 (SEQ ID NO:98), FUS+RNasin+RNA15 (SEQ ID NO:96), FUS+RNasin+RNA19 (SEQ ID NO:99), FUS+RNasin+RNA25 (SEQ ID NO:100), FUS+RNasin+RNA26 (SEQ ID NO:101), FUS+RNasin+RNA31 (SEQ ID NO:102), and FUS+RNasin+RNA32 (SEQ ID NO:103). While FUS alone and FUS+RNasin resulted in highly increased turbidity by 100 minutes, each of the strong inhibitors robustly prevented turbidity induction. The tested samples in FIG. 10B included FUS alone (solid line), FUS+RNasin (dotted line), FUS+RNasin+RNA1 (SEQ ID NO:104), FUS+RNasin+RNA2 (SEQ ID NO:105), FUS+RNasin+RNA9 (SEQ ID NO:106), FUS+RNasin+RNA10 (SEQ ID NO:107), FUS+RNasin+RNA11 (SEQ ID NO:108), FUS+RNasin+RNA13 (SEQ ID NO:109), FUS+RNasin+RNA14 (SEQ ID NO:110), FUS+RNasin+RNA17 (SEQ ID NO:111), FUS+RNasin+RNA27 (SEQ ID NO:112), FUS+RNasin+RNA28 (SEQ ID NO:113), FUS+RNasin+RNA29 (SEQ ID NO:114), and FUS+RNasin+RNA30 (SEQ ID NO:115). While FUS alone and FUS+RNasin resulted in highly increased turbidity by 100 minutes, each of the intermediate inhibitors prevented turbidity induction to an intermediate extent but not as well as the strong inhibitors. In order from most inhibition (least normalized turbidity) to least inhibition (most normalized turbidity) by 100 minutes, the intermediate inhibitors were ranked as RNA27, RNA17, RNA9≈RNA13≈RNA1≈RNA30≈RNA2 29≈RNA14≈RNA10, RNA30, RNA11.

FIG. 11 is a set of DIC images showing GST-TEV-FUS (5 μM) droplets formed in the presence of 20 μM of either RNA19 (upper left panel), RNA15 (upper middle panel), RNA9 (upper right panel), RNA8 (lower left panel), a control RNA (lower middle panel), or no RNA (lower right panel).

FIG. 12 is a set of DIC images showing GST-TEV-FUS (5 μM) droplets which were first formed, then subsequently exposed to 20 μM of either RNA19 (upper left panel), RNA15 (upper middle panel), RNA9 (upper right panel), RNA8 (lower left panel), a control RNA (lower middle panel), or no RNA (lower right panel).

FIG. 13(A-D) is a set of graphs showing RNA inhibitory and disaggregation activity depend on length and sequence of the RNA. GST-TEV-FUS protein was incubated with TEV protease (to cleave the GST-TEV motif), and 20 μM bONs were separately added at the beginning of the reaction (FIGS. 13A and 13B; prevention of aggregation) or at the end of the reaction (FIGS. 13C and 13D; disaggregation). Turbidity measurements were obtained over time. FUS with control RNA (solid line) served as a control in each experiment. In FIGS. 13A and 13C, the strong inhibitor RNA15 (SEQ ID NO:96; dotted line) was shortened from 48 nucleotides to 24 nucleotides to create RNA22 (SEQ ID NO:117; sometimes called “FUS+RNA15/2”; dashed line), then further modified to include an A-U point mutation to create RNA15/2(A-U) (SEQ ID NO:118; sometimes called “FUS+RNA15/2(A-U)”; dotted and dashed line). In FIGS. 13B and 13D, the 24 nucleotide RNA9 (SEQ ID NO:106; dotted line) was doubled to create the 48 nucleotide RNA9\hat{ }2 (SEQ ID NO: 119; sometimes called “FUS+RNA9\hat{ }2); dashed line).

FIG. 14(A-C) is a set of graphs showing optoFUS is a light-inducible model of FUS proteinopathy. FIG. 14A shows the nuclear/cytoplasmic ratio of optoFUS fluorescence signal in cells kept in darkness (left) or exposed to blue light (right). FIG. 14B shows the percentage of cells having cytoplasmic optoFUS assemblies in cells kept in darkness (left) or exposed to blue light (right). Cells having cytoplasmic optoFUS assemblies are represented by the dark gray segments of the bar graph, whereas cells having no cytoplasmic optoFUS assemblies are represented by the light gray segments of the bar graph. FIG. 14C shows that after exposing HEK294 cells expressing optoFUS to 8 h of either darkness (solid line) or blue light (dashed line) prior to fluorescence recovery after photobleaching (FRAP) analysis of optoFUS assemblies, light-induced optoFUS assemblies showed little fluorescence recovery following bleaching. Thus, FIG. 14C shows that optoFUS assemblies are static, immobile inclusions in cells and recapitulate pathological hallmarks.

FIG. 15 is a graph showing optoFUS inclusion formation observed in immunofluorescence experiments following treatment with control RNA oligonucleotides (left three bars) or RNAS1 (SEQ ID NO:98) (right three bars) at the indicated concentrations. Data were normalized to control RNA.

FIG. 16(A-B) is a set of graphs showing 2′OMe modified bait oligonucleotides (bONs) prevent optoFUS inclusions. HEK293 cells expressing optoFUS were pre-treated with unmodified non-binding control RNA (SEQ ID NO:121; gray bars), the unmodified RNAS1 (SEQ ID NO:98; black bars), the 2′OMe modified RNA2′S1 (SEQ ID NO:116; hatched bars), or the 2′OMe- and phosphorothioate bond (PS)-modified RNAPS-2′S1 (a PS-modified sequence of SEQ ID NO: 116; dotted bars) at the indicated concentrations prior to 8 hr exposure to blue light (FIG. 16A). Cells were then fixed and quantified for immunofluorescence. FIG. 16B shows the ratio of insoluble:soluble optoFUS protein identified in optoFUS-expressing HEK293 cells pre-treated with 1 μM of control RNA (left bar), unmodified RNAS1 (middle bar), or 2′OMe modified RNA2′S1 (right bar) prior to 8 hr blue light exposure. After the 8 hr exposure, cells were lysed, and cell lysates were separated into soluble and insoluble fractions. Fractions were separated on a gel, immunoblotted, and protein amounts were quantified by densitometry measurements.

FIG. 17 is a graph showing bait oligonucleotides (bONs) can disaggregate optoFUS inclusions. optoFUS-expressing HEK293 cells were exposed to 16 hr blue light, then treated with no RNA as a positive control (white bar), 1 μM unmodified non-binding control RNA (SEQ ID NO:121; gray bar), 1 μM unmodified RNAS1 (SEQ ID NO:98; black bar), or 1 μM 2′OMe modified RNA2′S1 (SEQ ID NO:116; hatched bar), then incubated in the dark for 6 hr. Cells were then fixed and quantified for immunofluorescence, and results were normalized to the positive control. Cells exposed to darkness throughout the experiment served as a negative control (dotted and hatched bar).

### DETAILED DESCRIPTION

The following description of the disclosure is provided as an enabling teaching of the disclosure in its best, currently known embodiments. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polynucleotide is disclosed and discussed and a number of modifications that can be made to the polynucleotide are discussed, specifically contemplated is each and every combination and permutation of the polynucleotide and the modifications that are possible unless specifically indicated to the contrary. Thus, if a class of polynucleotides A, B, and C are disclosed as well as a class of polynucleotides D, E, and F and an example of a combination polynucleotide, or, for example, a combination polynucleotide comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “can,” “may,” “optionally,” “can optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Use of the phrase “and/or” indicates that any one or any combination of a list of options can be used. For example, “A, B, and/or C” means “A”, or “B”, or “C”, or “A and B”, or “A and C”, or “B and C”, or “A and B and C”.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

A “control” is an alternative subject, sample, or set of values used in an experiment for comparison purposes. A control can be “positive” or “negative.” A control can also be a collection of values used as a standard applied to one or more subjects (e.g., a general number or average that is known and not identified in the method using a sample).

“Identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

As used herein, the terms “aggregate” or “aggregation” refer to a biological phenomenon in which a protein molecule attaches to another and accumulates to form a higher order complex such as a proteinaceous clump or fibril. Generally, protein aggregates contain a single type of protein, of which numerous copies attach and clump together. A protein aggregate can, but need not necessarily, deposit (e.g., on or in cells or tissue) as an insoluble complex. The term is intended to exclude small oligomeric clumps (10 copies of a protein molecule or less), as well as native hetero- and homo-polymeric proteins in which the polymeric form of the protein naturally contributes to the function of the protein (e.g., polymeric actin filaments). Incorrect three-dimensional protein folding (“misfolding”) is a known cause of protein aggregation. Numerous diseases, including many neurodegenerative diseases, are associated with protein aggregation. As used herein, the term “aggregate” and grammatical variations thereof, as it relates to proteins, is used interchangeably with the terms “inclusion,” “particle,” and grammatical variations thereof.

As used herein, the term “disaggregate” refers to the breaking down of one or more protein aggregates. As a protein aggregate contains numerous copies of a protein clumped together, disaggregation refers to a process of removing portions of the aggregated protein clump. Thus, as used herein, disaggregation refers to the removal of portions of an existing protein aggregate, such that after disaggregation, the result is a smaller protein aggregate clump or an absence of a protein aggregate clump altogether. Detection of aggregate size and changes thereto depend on the sensitivity of the equipment and techniques used to detect aggregate size. Thus, under one technique, a disaggregated clump may be undetectable, whereas under another technique, the same disaggregated clump may be detected as having a smaller size.

As used herein, the term “associated with a neurodegenerative disease,” as it relates to a nucleic acid-binding polypeptide, is intended to refer to the existence of a correlation between the nucleic acid-binding polypeptide and occurrence of the neurodegenerative disease which is sufficiently strong and researched, such that one of skill in the art would conclude that the nucleic acid-binding polypeptide (specifically, in vivo aggregation of the nucleic acid-binding polypeptide) likely plays a role in the risk, onset, progression, and/or exacerbation of the neurodegenerative disease. However, one of skill in the art would neither need to conclude that the nucleic acid-binding polypeptide plays the only role in the risk, onset, progression, and/or exacerbation of the neurodegenerative disease, nor that the particular neurodegenerative disease is always influenced or affected by the nucleic acid-binding protein.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Preventing” a disorder or unwanted physiological event in a subject refers specifically to the prevention of the occurrence of symptoms and/or their underlying cause, wherein the subject may or may not exhibit heightened susceptibility to the disorder or event. As used herein, preventing protein inclusion includes preventing or delaying the initiation of protein inclusion. The term further includes preventing a recurrence of one or more signs or symptoms of protein inclusion.

“Polynucleotide” refers to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (T) when the polynucleotide is RNA. Thus, a “polynucleotide” sequence can be represented by the sequential alphabetical representation of each base in a polynucleotide molecule.

“Peptide,” “protein,” and “polypeptide” are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

The term “TDP-43” refers herein to TAR DNA Binding Protein 43 kDa. TDP-43 is also known as TDP43, TARDBP, and ALS10. In some embodiments, the TDP-43 polypeptide or polynucleotide is that identified in one or more publicly available databases as follows: HGNC: 11571 Entrez Gene: 23435 Ensembl: ENSG00000120948 OMIM: 605078 UniProtKB: Q13148.

The term “Fus” refers herein to a FUS RNA binding protein. FUS is also known as “Heterogeneous Nuclear Ribonucleoprotein P2,” “Translocated In Liposarcoma Protein,” 75 KDa DNA-Pairing Protein,” “Fused In Sarcoma,” “Oncogene FUS,” “Oncogene TLS,” “POMP75,” and “TLS.” In some embodiments, the FUS polypeptide or polynucleotide is that identified in one or more publicly available databases as follows: HGNC: 4010 Entrez Gene: 2521 Ensembl: ENSG00000089280 OMIM: 137070 UniProtKB: P35637.

The term “Tau” refers herein to a Microtubule Associated Protein Tau. Tau is also known as “G Protein Betal/Gamm2 Subunit-Interacting Factor 1,” “Protein Phosphatase 1,” Neurofibrillary Tangle Protein,” “PPP1R103,” “FTDP-17,” “MTBT2,” “DDPAC,” “MSTD,” PPND,” “MAPTL,” and “MTBT1.” In some embodiments, the Tau polypeptide or polynucleotide is that identified in one or more publicly available databases as follows: HGNC: 6893 Entrez Gene: 4137 Ensembl: ENSG00000186868 OMIM: 157140 UniProtKB: P10636.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value” 10” is disclosed, then “about 10” is also disclosed.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The terms “treat,” “treating,” “treatment,” and grammatical variations thereof as used herein, include partially or completely delaying, curing, healing, alleviating, relieving, altering, remedying, ameliorating, improving, stabilizing, mitigating, and/or reducing the intensity or frequency of one or more diseases or conditions, symptoms of a disease or condition, or underlying causes of a disease or condition. Treatments according to the invention may be applied prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms.

### Compositions

It is understood that the polynucleotides of the present disclosure can be used in combination with the various compositions, methods, products, and applications disclosed herein.

The disclosure herein addresses needs in the art by providing for compositions and methods to treat one or more neurodegenerative diseases. To date, no currently approved drugs are available which can prevent protein aggregation or inclusion formation, disrupt existing protein aggregates or inclusions, and reduce the overall amount of protein aggregates or inclusions in a patient suffering from a neurodegenerative disease. The disclosed compositions and methods can achieve these outcomes by binding one or more polynucleotides to a neurodegenerative disease-associated nucleic acid-binding polypeptide to prevent aggregation or disrupt aggregates. Thus, the disclosed methods of using the polynucleotide compositions represent a major breakthrough in the advancement of neurodegenerative disease treatments.

Disclosed herein are compositions comprising a polynucleotide that binds a nucleic acid-binding polypeptide, wherein the nucleic acid-binding polypeptide can aggregate within or outside cells and is associated with a neurodegenerative disease.

The nucleic acid-binding polypeptide can be any polypeptide that can or is known to aggregate in cells and is associated with a neurodegenerative disease. Deposited aggregates can be toxic to cells and tissues. An aggregate generally comprises numerous molecules of the same protein, but can be comprised of a mixture of different protein molecules. The protein molecules of an aggregate are typically arranged in a regular three-dimensional structure and frequently are bonded together via hydrophobic interaction, although other intermolecular forces can further stabilize the interaction. A polypeptide can or is known to aggregate in cells when it is observed to form an aggregate in a subject (e.g., in a post-mortem tissue analysis), or in a cell- or tissue-based assay under physiological conditions. Generally, the cell or tissue used in the assay natively expresses the polypeptide in vivo. For instance, the polypeptide TDP-43 is a human polypeptide which can be natively expressed in human neuronal cells, and has been observed to form protein aggregates in both cell-based assays and in post-mortem brain autopsies. Typically, the polypeptide aggregate can deposit on cells of the nervous system such as glial cells and/or neuronal cells.

The nucleic acid-binding polypeptide can have a number of various attributes. In some embodiments, the nucleic acid-binding polypeptide comprises a RNA-binding domain (sometimes referred to as a RNA-recognition motifs or RRM). In some embodiments, the nucleic acid-binding polypeptide comprises a C-terminal glycine-rich, prion-like/low complexity domain (LCD; sometimes referred to as an intrinsically-disordered region (IDR)).

In some embodiments, the nucleic acid-binding polypeptide has an increased rate of aggregation when the concentration of free polynucleotides is reduced. As used herein, the term “free polynucleotide” (encompassing, for example, “free RNA,” and “free DNA”) when used in reference to intracellular polynucleotides, refers to polynucleotides present in the cell and readily available for binding (e.g., not sequestered or sterically blocked) to the nucleic acid-binding protein, but is not bound by the nucleic acid-binding protein. Free polynucleotides can be bound by other factors (e.g., other proteins) if the polynucleotides remain readily available for binding to the nucleic acid-binding protein. Conversely, a polynucleotide which is sequestered, sterically blocked from further binding, housed within a vacuole, incorporated into a large supramolecular complex, or otherwise incapable of readily binding to the nucleic-acid binding protein is not a free polynucleotide. Reduction of free polynucleotides can be below average cellular or physiological levels. Average cellular and physiological levels of free polynucleotides differ between organisms, tissue types, and cell types and are readily determinable by those skilled in the art, but in some embodiments can be roughly 7.5 μg/μL free RNA in the nucleus and roughly 0.2 μg/μL free RNA in the cytoplasm. Average levels of free polynucleotides can be determined empirically from a control sample or can be a collection of values used as a standard applied to one or more subjects (e.g., a general number or average that is known and not identified in the method using a sample). In some embodiments, the nucleic acid-binding polypeptide can aggregate intracellularly in the cytoplasm. In some embodiments, the nucleic acid-binding polypeptide can aggregate and deposit on cells such as neuronal cells. In some embodiments, aggregation of the nucleic acid-binding polypeptide can cause, exacerbate, and/or accelerate a neurodegenerative disease or a symptom of a neurodegenerative disease.

In some embodiments, binding of the polynucleotide to the nucleic acid-binding polypeptide results in a complex which associates with stress granules or stress granule components, whereas lack of binding of the polynucleotide to the nucleic acid-binding polypeptide results in aggregated nucleic acid-binding polypeptide which does not associate with stress granules or stress granule components. In some embodiments, binding of the polynucleotide to the nucleic acid-binding polypeptide results in a complex which is soluble under physiological conditions (e.g., no appreciable aggregation of the nucleic acid-binding polypeptide occurs upon binding to the polynucleotide). In some embodiments, binding of the polynucleotide to the nucleic acid-binding polypeptide prevents, delays, or reverses aggregation of the nucleic acid-binding polypeptide.

In some embodiments, the nucleic acid-binding polypeptide specifically binds nucleic acids. As used herein, the term “specifically binds” refers to a binding reaction in which a first molecule that “specifically binds” a second molecule has an affinity constant (Ka) greater than about 10² M⁻¹ (e.g., 10³ M⁻¹, 10⁴ M⁻¹, 10⁵ M⁻¹, 10⁶ M⁻¹, 10⁷ M⁻¹, 10⁸ M⁻¹, and 10⁹ M⁻¹ or more) with that second molecule.

In some embodiments, the nucleic acid-binding polypeptide preferentially binds UG-rich RNA. As used herein, the term “preferentially binds,” as used in reference to the binding of a nucleic acid-binding polypeptide and a nucleic acid, refers to a binding reaction in which the nucleic acid-binding polypeptide binds the nucleic acid comprising the stated characteristic with an affinity constant (Ka) which is greater compared to the affinity constant when binding to an RNA which lacks the stated characteristic.

In some embodiments, the nucleic acid-binding polypeptide can be any one or more of TDP-43, FUS, HNRNPA1, HNRNPA2/B1, EWSR1, TIA-1, TTP, TAF15, MATR3, ATXN2, OPTN, Tau, HTT, and biologically active fragments and variants thereof. In some embodiments, the nucleic acid-binding polypeptide comprises TDP-43 or a biologically active fragment or variant thereof, or FUS or a biologically active fragment or variant thereof.

TABLE 1
Example RNA and/or DNA
binding polypeptides which can be aggregated in
neurodegenerative diseases.

| RNA/DNA Binding Protein | Associated Disease |
|---|---|
| TDP-43 | ALS, FTD, CTE, AD, HD |
| FUS | ALS, FTD |
| HNRNPA1 | ALS, MSP |
| HNRNPA2/B1 | ALS, MSP |
| EWSR1 | ALS |
| TIA-1 | ALS |
| TTP | ALS, FTD, AD |
| TAF15 | ALS, FTD |
| ATXN2 | ALS |
| OPTN | ALS |
| MART3 | ALS |
| ADAR1 | ALS |
| ADAR2 | ALS |
| ADAR3 | ALS |
| Potential RNA Binding Proteins | Associated Disease |
| Tau | AD, TBI, FTD, CBD |
| HTT | HD |

ALS: Amyotrophic Lateral Sclerosis
MSP: Multisystem Proteinopathy
FTD: Frontotemporal Dementia
AD: Alzheimer's Disease
CTE: Chronic Traumatic Encephalopathy
TBI: Traumatic Brain Injury
CBD: Cortical Basal Degeneration
HD: Huntington's Disease

In some embodiments, the nucleic acid-binding polypeptide can comprise an amino acid sequence which is at least 70% identical to SEQ ID NO:1. In some embodiments, the nucleic acid-binding polypeptide is a polypeptide comprising an amino acid sequence which is at least 75%, at least 80%, at least 85%, at least 90%, at least 95%, at least 98%, or at least 99% identical to SEQ ID NO:1. In some embodiments, the nucleic acid-binding polypeptide is a polypeptide comprising SEQ ID NO:1.

In some embodiments, the nucleic acid-binding polypeptide can comprise an amino acid sequence which is at least 70% identical to SEQ ID NO:2. In some embodiments, the nucleic acid-binding polypeptide is a polypeptide comprising an amino acid sequence which is at least 75%, at least 80%, at least 85%, at least 90%, at least 95%, at least 98%, or at least 99% identical to SEQ ID NO:2. In some embodiments, the nucleic acid-binding polypeptide is a polypeptide comprising SEQ ID NO:2.

In some embodiments, the nucleic acid-binding polypeptide can comprise an amino acid sequence which is at least 70% identical to SEQ ID NO:3. In some embodiments, the nucleic acid-binding polypeptide is a polypeptide comprising an amino acid sequence which is at least 75%, at least 80%, at least 85%, at least 90%, at least 95%, at least 98%, or at least 99% identical to SEQ ID NO:3. In some embodiments, the nucleic acid-binding polypeptide is a polypeptide comprising SEQ ID NO:3.

The nucleic acid-binding polypeptide is associated with one or more neurodegenerative diseases. Neurodegenerative diseases include, but not limited to, Limbic-predominant Age-related TDP-43 Encephalopathy (LATE), Alzheimer's disease (AD), dementia, Parkinson's disease (PD), Prion disease, Motor neurone diseases (MND), Huntington's disease (HD), Spinocerebellar ataxia (SCA), Spinal muscular atrophy (SMA), Multisystem Proteinopathy, Amyotrophic Lateral Sclerosis (ALS), Frontotemporal Dementia, Chronic Traumatic Encephalopathy, Traumatic Brain Injury, Cortical Basal Degeneration, Friedreich's ataxia, Lewy body disease, Alper's Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Shy-Drager Syndrome, Opsoclonus Myoclonus, Progressive Multifocal Leukoencephalopathy, Striatonigral Degeneration, Spongiform Encephalopathy, Creutzfeldt-Jakob Disease, Primary Progressive Aphasia, and Progressive Supranuclear Palsy. In some embodiments, the neurodegenerative disease can comprise Multisystem Proteinopathy, Amyotrophic Lateral Sclerosis, Frontotemporal Dementia, Alzheimer's Disease, Chronic Traumatic Encephalopathy, Traumatic Brain Injury, Cortical Basal Degeneration, and Huntington's Disease. A patient may suffer from any one or more neurodegenerative diseases. In some embodiments, the neurodegenerative disease can comprise Limbic-predominant Age-related TDP-43 Encephalopathy (LATE), Alzheimer's disease (AD), Amyotrophic Lateral Sclerosis or Frontotemporal Dementia. In some embodiments, the neurodegenerative disease can comprise Amyotrophic Lateral Sclerosis or Frontotemporal Dementia.

An array of polynucleotide molecules can be compatible with the disclosed technology. In some embodiments, the polynucleotide in the composition comprises RNA, DNA, or combinations thereof. The polynucleotide can be either single-stranded or double-stranded. In some embodiments, a single-stranded polynucleotide is selected. In some embodiments, the polynucleotide comprises RNA. In some embodiments, the polynucleotide comprises DNA, particularly a modified DNA. In some embodiments, the polynucleotide can comprise a synthetic polynucleotide (e.g., a synthetic RNA). By “synthetic,” it is meant that the polynucleotide is non-naturally occurring and formed by laboratory, commercial, or industrial methods.

The polynucleotide can be a modified polynucleotide. The polynucleotide can contain one or more modifications, for example, methylation, deamination, thiolation, alkylation, pegylation, phosphorothioate bonds, 2′-O-methylation, and numerous others known in the art. Modifications can be present not only on synthetic polynucleotides, but also on polynucleotides from natural sources or derived from natural sources (thereby resulting in a “non-natural” polynucleotide).

In some embodiments, the composition further comprises one or more additional polynucleotides. In some embodiments, the composition can comprise a plurality of polynucleotides.

Generally, the polynucleotide is at least 2 nucleotides long. In some embodiments, the polynucleotide is at least 3, at least 4, at least 5, at least 6, at least 7, at least 8, at least 9, at least 10, at least 12, at least 15, at least 20, at least 25, at least 30, or at least 50 nucleotides long. In some embodiments, the polynucleotide is at least 100, at least 150, at least 200, at least 250, at least 300, at least 350, at least 400, at least 450, or at least 500 nucleotides long. In some embodiments, the polynucleotide is at least 24 nucleotides long.

In some embodiments, the polynucleotide comprises 500 nucleotides or less. In some embodiments, the polynucleotide comprises 400, 300, 250, 200, or 150 nucleotides or less. In some embodiments, the polynucleotide comprises 100 nucleotides or less. In some embodiments, the polynucleotide comprises 75 nucleotides or less. In some embodiments, the polynucleotide comprises 50 nucleotides or less.

The polynucleotide can have a variety of length ranges. For instance, the polynucleotide can be from 2 to 500 nucleotides long. In some embodiments, the polynucleotide can range from 5 to 250 nucleotides long, from 10 to 100 nucleotides long, from 15 to 75 nucleotides long, or from 20 to 50 nucleotides long. In some embodiments, the polynucleotide can range from 20 to 45 nucleotides long, from 21 to 44 nucleotides long, from 22 to 43 nucleotides long, from 23 to 42 nucleotides long, from 24 to 41 nucleotides long, or from 25 to 40 nucleotides long. In some embodiments, the polynucleotide can range from 5 to 20 nucleotides long, or from 10 to 20 nucleotides long, or from 10 to 15 nucleotides long.

In some embodiments, the polynucleotide comprises a uracil- and guanine-rich nucleic acid sequence, or a thymine- and guanine-rich nucleic acid sequence. In some embodiments, the uracil- and guanine-rich nucleic acid sequence or the thymine- and guanine-rich nucleic acid sequence is at least 24 bp long. In some embodiments, the uracil- and guanine-rich nucleic acid sequence comprises at least 55%, at least 60%, at least 70%, at least 80%, at least 90%, at least 95%, or at least 99% uracil and guanine nucleotides. In some embodiments, the thymine- and guanine-rich nucleic acid sequence comprises at least 55%, at least 60%, at least 70%, at least 80%, at least 90%, at least 95%, or at least 99% thymine and guanine nucleotides. In some embodiments, the polynucleotide comprises a GGUG or a GGTG sequence, which are known to be binding recognition sequences for nucleic acid-binding polypeptides. Other binding recognition sequences for nucleic acid-binding polypeptides include, but are not limited to, (TG)ₙ wherein n is an integer from 6 to 10 representing the number of repeats of the immediately preceding sequence in parentheses, and (TG)ₙTA(TG)ₘ wherein n and m are both integers separately ranging from 0 to 5 and represent the number of repeats of the immediately preceding sequence in parentheses (Colombrita et al., J. Biol. Chem., (2012), 287, 15635-15647. Further binding recognition sequences for nucleic acid-binding polypeptides include, but are not limited to, AGN(A/C)(A/G)NNNUG, G(A/C)UN(U/C)(G/C)A(A/G)UG, and GCU(U/G)C(C/G)A(G/C)C, wherein two nucleotides in parentheses separated by a “/” refer to selection of either one of the two nucleotides, and wherein N refers to any nucleotide (Choi et al., Biotechniques, (2017), 62:28-33).

The polynucleotide can comprise a single sequence, or alternatively can comprise a sequence which is repeated one or more times. Polynucleotide sequences which are well-suited for use singly or as repeated motifs in a polynucleotide that binds a nucleic acid-binding polypeptide include SEQ ID NO:33-56 and can be repeated “n” times, wherein the subscript “n” is an integer from 1 to 100. For example, the sequence (ATGTGTGTGTGT)ₙ (SEQ ID NO:33) can be used as a polynucleotide that binds a nucleic acid-binding polypeptide in the form of ATGTGTGTGTGT (n=1), ATGTGTGTGTGTATGTGTGTGTGT (n=2), ATGTGTGTGTGTATGTGTGTGTGTATGTGTGTGTGT (n=3), and so on. In some embodiments, the polynucleotide can comprise a sequence which is repeated once. In some embodiments, the polynucleotide can comprise a sequence which is repeated twice, three times, four times, or five times. In some embodiments, the polynucleotide can comprise a sequence which is repeated more than five times. In some embodiments, “n” can be any integer from 1 to 100, or from 1 to 50, or from 1 to 25, or from 1 to 10, from 1 to 5, from 1 to 4, from 1 to 3, or from 1 to 2.

In some embodiments, the polynucleotide can comprise a nucleic acid sequence which is at least 70% identical to any one or more of SEQ ID NO:10 through SEQ ID NO:84 or SEQ ID NO:96-119. In some embodiments, the polynucleotide can comprise a nucleic acid sequence which is at least 70% identical to any one or more of SEQ ID NO:10 through SEQ ID NO:84. In some embodiments, the polynucleotide can comprise a nucleic acid sequence 15 which is at least 70% identical to any one or more of SEQ ID NO:96 through SEQ ID NO:119. In some embodiments, the polynucleotide can comprise a nucleic acid sequence which is at least 75%, at least 80%, at least 85%, at least 90%, at least 95%, at least 98%, or at least 99% identical to any one or more of the sequences described in this paragraph. In some embodiments, the polynucleotide is a polynucleotide comprising any one or more of the sequences described in this paragraph.

In some embodiments, the selected polynucleotide comprises any one or more of SEQ ID NO:62 through SEQ ID NO:84. A polynucleotide comprising any one or more of SEQ ID NO:62 through SEQ ID NO:84 can further comprise any one or more of SEQ ID NO:85 through SEQ ID NO:95. SEQ ID NO:85 through SEQ ID NO:95 are useful for forming stem secondary structures, for instance in a stem loop. An array of combinations are possible, which include non-limiting examples such as a polynucleotide comprising any of the following:
SEQ ID NO:85, SEQ ID NO:62, and SEQ ID NO:86, in that order;
SEQ ID NO:87, SEQ ID NO:63, and SEQ ID NO:88, in that order;
SEQ ID NO:92, SEQ ID NO:64, and SEQ ID NO:92, in that order;
SEQ ID NO:92, SEQ ID NO:65, and SEQ ID NO:92, in that order;
SEQ ID NO:92, SEQ ID NO:66, and SEQ ID NO:92, in that order;
SEQ ID NO:92, SEQ ID NO:67, and SEQ ID NO:92, in that order;
SEQ ID NO:92, SEQ ID NO:68, and SEQ ID NO:92, in that order;
SEQ ID NO:92, SEQ ID NO:69, and SEQ ID NO:92, in that order;
SEQ ID NO:92, SEQ ID NO:70, and SEQ ID NO:92, in that order;
SEQ ID NO:92, SEQ ID NO:71, and SEQ ID NO:92, in that order;
SEQ ID NO:92, SEQ ID NO:72, and SEQ ID NO:92, in that order;
SEQ ID NO:92, SEQ ID NO:73, and SEQ ID NO:92, in that order;
SEQ ID NO:92, UGUGUGUG, and SEQ ID NO:92, in that order;
SEQ ID NO:92, UGUGUG, and SEQ ID NO:92, in that order;
SEQ ID NO:89, SEQ ID NO:74, and SEQ ID NO:89, in that order;
SEQ ID NO:90, SEQ ID NO:74, and SEQ ID NO:90, in that order;
SEQ ID NO:91, SEQ ID NO:74, and SEQ ID NO:91, in that order;
SEQ ID NO:92, SEQ ID NO:74, and SEQ ID NO:92, in that order;
SEQ ID NO:93, SEQ ID NO:74, and SEQ ID NO:93, in that order;
SEQ ID NO:94, SEQ ID NO:74, and SEQ ID NO:94, in that order;
SEQ ID NO:95, SEQ ID NO:74, and SEQ ID NO:95, in that order;
CGCGCGCG, SEQ ID NO:74, and CGCGCGCG, in that order;
CGCGCG, SEQ ID NO:74, and CGCGCG, in that order;
CGCG, SEQ ID NO:74, and CGCG, in that order;
CG, SEQ ID NO:74, and CG, in that order;
SEQ ID NO:92, SEQ ID NO:75, and SEQ ID NO:92, in that order;
SEQ ID NO:92, SEQ ID NO:76, and SEQ ID NO:92, in that order;
SEQ ID NO:92, SEQ ID NO:77, and SEQ ID NO:92, in that order;
SEQ ID NO:92, SEQ ID NO:78, and SEQ ID NO:92, in that order;
SEQ ID NO:92, SEQ ID NO:79, and SEQ ID NO:92, in that order;
SEQ ID NO:92, SEQ ID NO:80, and SEQ ID NO:92, in that order;
SEQ ID NO:92, SEQ ID NO:81, and SEQ ID NO:92, in that order;
SEQ ID NO:92, SEQ ID NO:82, and SEQ ID NO:92, in that order;
SEQ ID NO:92, SEQ ID NO:83, and SEQ ID NO:92, in that order;
SEQ ID NO:92, SEQ ID NO:84, and SEQ ID NO:92, in that order;
SEQ ID NO:92, TGTGTGTG, and SEQ ID NO:92, in that order; or
SEQ ID NO:92, TGTGTG, and SEQ ID NO:92, in that order.

Where the selected polynucleotide comprises two or more of the disclosed sequences, 100 or less intervening nucleotides can be between the two or more disclosed sequences. In some embodiments, 90 or less, 80 or less, 70 or less, 60 or less, 50 or less, 40 or less, 30 or less, 20 or less, or 10 or less intervening nucleotides can be between the two or more disclosed sequences. In some embodiments, 7 or less, 5 or less, 3 or less, 2 or less, 1, or 0 intervening nucleotides can be between the two or more disclosed sequences. Where the selected polynucleotide comprises three or more of the disclosed sequences, neither the number nor the sequence of the intervening nucleotides between the disclosed sequences need be the same, As a non-limiting example, the selected polynucleotide can comprise SEQ ID NO:85, followed by the intervening sequence “AGCG”, followed by SEQ ID NO:62, followed by the intervening sequence “GGGGCC”, and followed by SEQ ID NO:86, in that order.

In some embodiments, the selected polynucleotide comprises any one or more of SEQ ID NO:10 through SEQ ID NO:61. A polynucleotide comprising any one or more of SEQ ID NO:10 through SEQ ID NO:61 can further comprise any one or more of SEQ ID NO:85 through SEQ ID NO:95. An array of combinations are possible.

In some embodiments, the selected polynucleotide comprises any one or more of SEQ ID NO:10, SEQ ID NO:11, SEQ ID NO:12, SEQ ID NO:13, SEQ ID NO:14, and SEQ ID NO:59.

In some embodiments, the selected polynucleotide comprises any one or more of SEQ ID NO:96, SEQ ID NO:97, SEQ ID NO:98, SEQ ID NO:99, SEQ ID NO:100, SEQ ID NO:101, SEQ ID NO:102, or SEQ ID NO:103. In some embodiments, the selected polynucleotide comprises any one or more of SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:117, SEQ ID NO:118, and SEQ ID NO:119. In some embodiments, the selected polynucleotide comprises any one or more of SEQ ID NO:98 and SEQ ID NO:116.

The polynucleotide can have secondary structure or be further bound to additional components. For example, the polynucleotide can comprise a region of paired nucleic acids (base pairs), for instance in the stem portion of a stem loop. In some embodiments, the polynucleotide comprises a secondary structure. In some embodiments, the secondary structure (e.g., stem loop structure) facilitates binding of the polynucleotide to the nucleic acid-binding polypeptide.

Numerous polynucleotides which bind to the nucleic acid-binding polypeptides disclosed herein are known in the art. For example, polynucleotides which bind to the nucleic acid-binding polypeptide TDP-43 are disclosed in Sephton et al., J. Biol. Chem., (2011) 286(2):1204-1215.

Also disclosed herein are pharmaceutical compositions comprising a therapeutically effective amount of a pharmaceutically acceptable excipient and a polynucleotide that binds a nucleic acid-binding polypeptide, wherein the nucleic acid-binding polypeptide can aggregate in cells and is associated with a neurodegenerative disease. In some embodiments, the polynucleotide is present in a therapeutically effective amount to treat a neurodegenerative disease. Suitable excipients include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, antinfective agents, suspending agents, wetting agents, viscosity modifiers, tonicity agents, stabilizing agents, and other components and combinations thereof. Suitable pharmaceutically acceptable excipients are preferably selected from materials which are generally recognized as safe (GRAS), and may be administered to an individual without causing undesirable biological side effects or unwanted interactions. Suitable excipients and their formulations are described in Remington's Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions. Typically, the components of the formulation are dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol. In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers. In some embodiments, the formulation can be distributed or packaged in a liquid form, or alternatively, as a solid, obtained, for example by lyophilization of a suitable liquid formulation, which can be reconstituted with an appropriate carrier or diluent prior to administration. The pharmaceutical compositions comprise a polynucleotide that selectively binds a nucleic acid-binding polypeptide, wherein the nucleic acid-binding polypeptide is capable of intracellular aggregation and is associated with a neurodegenerative disease in a therapeutically effective amount sufficient to treat a neurodegenerative disease. The pharmaceutical compositions can be formulated for medical and/or veterinary use.

Also disclosed herein are polynucleotides comprising a nucleic acid sequence which is at least 70% identical to any one or more of SEQ ID NO:10 through SEQ ID NO:84 and SEQ ID NO:96-119. In some embodiments, the polynucleotide can comprise a nucleic acid sequence which is at least 75%, at least 80%, at least 85%, at least 90%, at least 95%, at least 98%, or at least 99% identical to any one or more of SEQ ID NO:10 through SEQ ID NO:84 and SEQ ID NO:96-119. In some embodiments, the polynucleotide is a polynucleotide comprising any one or more of SEQ ID NO:10 through SEQ ID NO:84 and SEQ ID NO:96-119.

In some embodiments, the polynucleotide can comprise a nucleic acid sequence which is at least 70% identical to any one or more of SEQ ID NO:10-14, 33-56, and 61-84. In some embodiments, the polynucleotide can comprise a nucleic acid sequence which is at least 75%, at least 80%, at least 85%, at least 90%, at least 95%, at least 98%, or at least 99% identical to any one or more of SEQ ID NO:10-14, 33-56, and 61-84. In some embodiments, the polynucleotide is a polynucleotide comprising any one or more of SEQ ID NO:10-14, 33-56, and 61-84.

Also disclosed herein are polynucleotides comprising a nucleic acid sequence which is at least 70% identical to any one or more of SEQ ID NO:10, SEQ ID NO:11, SEQ ID NO:12, SEQ ID NO:13, and SEQ ID NO:14. In some embodiments, the polynucleotide can comprise a nucleic acid sequence which is at least 75%, at least 80%, at least 85%, at least 90%, at least 95%, at least 98%, or at least 99% identical to any one or more of SEQ ID NO:10, SEQ ID NO:11, SEQ ID NO:12, SEQ ID NO:13, and SEQ ID NO:14. In some embodiments, the polynucleotide is a polynucleotide comprising any one or more of SEQ ID NO:10, SEQ ID NO:11, SEQ ID NO:12, SEQ ID NO:13, and SEQ ID NO:14.

Also disclosed herein are polynucleotides comprising a nucleic acid sequence which is at least 70% identical to any one or more of SEQ ID NO:96, SEQ ID NO:97, SEQ ID NO:98, SEQ ID NO:99, SEQ ID NO:100, SEQ ID NO:101, SEQ ID NO:102, or SEQ ID NO:103. In some embodiments, the polynucleotide can comprise a nucleic acid sequence which is at least 75%, at least 80%, at least 85%, at least 90%, at least 95%, at least 98%, or at least 99% identical to any one or more of SEQ ID NO:96, SEQ ID NO:97, SEQ ID NO:98, SEQ ID NO:99, SEQ ID NO:100, SEQ ID NO:101, SEQ ID NO:102, or SEQ ID NO:103. In some embodiments, the polynucleotide is a polynucleotide comprising any one or more of SEQ ID NO:96, SEQ ID NO:97, SEQ ID NO:98, SEQ ID NO:99, SEQ ID NO:100, SEQ ID NO:101, SEQ ID NO:102, or SEQ ID NO:103.

Also disclosed herein are polynucleotides comprising a nucleic acid sequence which is at least 70% identical to any one or more of SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:117, SEQ ID NO:118, and SEQ ID NO:119. In some embodiments, the polynucleotide can comprise a nucleic acid sequence which is at least 75%, at least 80%, at least 85%, at least 90%, at least 95%, at least 98%, or at least 99% identical to any one or more of SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:117, SEQ ID NO:118, and SEQ ID NO:119. In some embodiments, the polynucleotide is a polynucleotide comprising any one or more of SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:117, SEQ ID NO:118, and SEQ ID NO:119.

Also disclosed herein are polynucleotides comprising a nucleic acid sequence which is at least 70% identical to any one or more of SEQ ID NO:98 and SEQ ID NO:116. In some embodiments, the polynucleotide can comprise a nucleic acid sequence which is at least 75%, at least 80%, at least 85%, at least 90%, at least 95%, at least 98%, or at least 99% identical to any one or more of SEQ ID NO:98 and SEQ ID NO:116. In some embodiments, the polynucleotide is a polynucleotide comprising any one or more of SEQ ID NO:98 and SEQ ID NO:116.

Also disclosed herein are polynucleotides comprising a nucleic acid sequence which is at least 70% identical to any one or more of SEQ ID NO:99-101 and 106-119. In some embodiments, the polynucleotide can comprise a nucleic acid sequence which is at least 75%, at least 80%, at least 85%, at least 90%, at least 95%, at least 98%, or at least 99% identical to any one or more of SEQ ID NO:99-101 and 106-119. In some embodiments, the polynucleotide is a polynucleotide comprising any one or more of SEQ ID NO:99-101 and 106-119.

Also disclosed herein is a composition to model TDP-43 protein aggregation comprising a nucleotide sequence encoding a chimeric polypeptide, comprising: a first nucleotide sequence encoding a light-induced oligomerization domain and a second nucleotide sequence encoding a TDP-43 polypeptide. The TDP-43 polypeptide can be full-length TDP-43 polypeptide, or can in some embodiments comprise the TDP-43 Low Complexity Domain (LCD), the TDP-43 RNA-Recognition Motif (RRM), or both. The composition to model TDP-43 protein aggregation can comprise any of the TDP43 modeling constructs (e.g., optoTDP43) disclosed in PCT/US2018/021335, which is incorporated by reference herein in its entirety. In some embodiments, the light-induced oligomerization domain is selected from the group consisting of CRYPHR, CRY2OLIG, NcVVD, NcVVDY50W, NcLOV, and VfAU1LOV. In some embodiments, the first and second nucleotide sequences are comprised within an expression vector. Also disclosed is a cell comprising a nucleotide sequence encoding a chimeric polypeptide, comprising: a first nucleotide sequence encoding a light-induced oligomerization domain and a second nucleotide sequence encoding a TDP-43 polypeptide. In some embodiments, the TDP-43 polypeptide comprises a full-length TDP-43 polypeptide.

Also disclosed herein is a composition to model FUS protein aggregation comprising a nucleotide sequence encoding a chimeric polypeptide, comprising: a first nucleotide sequence encoding a light-induced oligomerization domain and a second nucleotide sequence encoding a FUS polypeptide. The FUS polypeptide can be full-length FUS polypeptide, or can comprise the FUS Low Complexity Domain (LCD), the FUS RNA-Recognition Motif (RRM), or both. In some embodiments, the light-induced oligomerization domain is selected from the group consisting of CRYPHR, CRY2OLIG, NcVVD, NcVVDY50W, NcLOV, and VfAU1LOV. In some embodiments, the first and second nucleotide sequences are comprised within an expression vector. Also disclosed is a cell comprising a nucleotide sequence encoding a chimeric polypeptide, comprising: a first nucleotide sequence encoding a light-induced oligomerization domain and a second nucleotide sequence encoding a FUS polypeptide.

In some embodiments, the FUS polypeptide is comprised within a full-length FUS polypeptide. In some embodiments, the FUS polypeptide is comprised within a sequence which is at least 70% identical to SEQ ID NO:5. In some embodiments, the FUS polypeptide is comprised within a sequence which is at least 75%, at least 80%, at least 85%, at least 90%, at least 95%, at least 98%, or at least 99% identical to SEQ ID NO:5. In some embodiments, the polynucleotide is a polypeptide comprising SEQ ID NO:5.

### Methods of Treatment

Also disclosed herein are methods to treat and/or prevent a neurodegenerative disease comprising administering to a subject with a neurodegenerative disease a therapeutically effective amount of a polynucleotide that binds a nucleic acid-binding polypeptide, wherein the nucleic acid-binding polypeptide can aggregate in cells and is associated with a neurodegenerative disease. The nucleic acid-binding polypeptide can be any herein disclosed nucleic acid-binding polypeptide. The polynucleotide can be any herein disclosed polynucleotide, but can also include, in some embodiments, a naturally occurring polynucleotide, or a polynucleotide derived from a natural source (e.g., cleaved from a naturally occurring polynucleotide).

The methods are useful for treating and/or preventing a variety of neurodegenerative diseases. For instance, a subject may suffer from, be diagnosed with, or be suspected of having any one or more neurodegenerative diseases including, but not limited to, Limbic-predominant Age-related TDP-43 Encephalopathy (LATE), Alzheimer's disease (AD), dementia, Parkinson's disease (PD), Prion disease, Motor neurone diseases (MND), Huntington's disease (HD), Spinocerebellar ataxia (SCA), Spinal muscular atrophy (SMA), Multisystem Proteinopathy, Amyotrophic Lateral Sclerosis (ALS), Frontotemporal Dementia, Chronic Traumatic Encephalopathy, Traumatic Brain Injury, Cortical Basal Degeneration, Friedreich's ataxia, Lewy body disease, Alper's Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Shy-Drager Syndrome, Opsoclonus Myoclonus, Progressive Multifocal Leukoencephalopathy, Striatonigral Degeneration, Spongiform Encephalopathy, Creutzfeldt-Jakob Disease, Primary Progressive Aphasia, and Progressive Supranuclear Palsy. In some embodiments, the neurodegenerative disease can comprise Multisystem Proteinopathy, Amyotrophic Lateral Sclerosis, Frontotemporal Dementia, Alzheimer's Disease, Chronic Traumatic Encephalopathy, Traumatic Brain Injury, Cortical Basal Degeneration, and Huntington's Disease. A patient may suffer from any one or more neurodegenerative diseases. In some embodiments, the neurodegenerative disease can comprise Limbic-predominant Age-related TDP-43 Encephalopathy (LATE), Alzheimer's disease (AD), Amyotrophic Lateral Sclerosis or Frontotemporal Dementia. In some embodiments, the neurodegenerative disease can comprise Amyotrophic Lateral Sclerosis or Frontotemporal Dementia.

The methods can improve a range of physical, mental, and emotional attributes of the treated subject. The subject can show an improvement in one or more symptoms of a neurodegenerative disease. Such improvements include, but are not limited to, improved physical abilities such as fine motor skills (e.g., writing and typing, grasping small objects, cutting, pointing, etc.), or gross motor skills (e.g., walking, balance, jumping, standing up, throwing); improved sensations such as decreased tingling and/or increased sensitivity in extremities, reduced sensation of muscle weakness or rigidity, and reduced tremors or pain; improved cognitive abilities such as increased alertness, reduced memory loss/improved memory recall, increased cognitive comprehension, improved speech and sleep, improved puzzle-solving abilities, increased focus; and improved behavioral performance such as decreased apathy, depression, agitation, or anxiety, and improved mood and general contentment.

In some embodiments, the methods treats or prevents a neurodegenerative disease by reducing the rate of aggregation of the nucleic acid-binding polypeptide in the subject (e.g., reducing the rate of formation of protein inclusions). In some embodiments, the methods treats a neurodegenerative disease by reducing the amount of aggregate of the nucleic acid-binding polypeptide in the subject (e.g., reducing the amount of protein inclusions). In some embodiments, the method prevents aggregation of the nucleic acid-binding polypeptide in the subject. Thus, the methods can reduce and/or prevent formation of pathological inclusions in cells of a subject. For instance, the methods can treat and/or prevent pathological phase separation and aggregation of one or more nucleic acid-binding polypeptides.

In some or further embodiments, the methods can disaggregate existing protein aggregates. Thus, the methods can reduce the amount of existing protein aggregates prior to beginning the methods. This can be important for patients experiencing neurodegenerative disease symptoms, as such patients are likely to have existing protein aggregates. Disaggregation of existing aggregates can be, but need not necessarily be, in addition to prevention or reduction of further aggregate formation.

The methods can generate neuroprotective results when performed in a subject. As used herein, the term “neuroprotective” refers to maintaining or improving existing neurological function in the target neurological organ or tissue (e.g., nerve, spinal cord), or can refer to maintaining or improving the rate or overall amount of neuronal cell death in target neuronal cells. For example, “neuroprotective” can refer to slowing the rate of nerve tissue destruction, deterioration, or malfunction, slowing the rate of neuronal cell death, reducing the rate at which nerve conduction speed slows, etc. In some embodiments, the methods can generate at least 5%, at least 10%, at least 20%, or at least 25% or more neuroprotective improvement, as compared to a control.

The subject can be any mammalian subject, for example a human, dog, cow, horse, mouse, rabbit, etc. In some embodiments, the subject is a primate, particularly a human. The subject can be a male or female of any age, race, creed, ethnicity, socio-economic status, or other general classifiers. The subject can be diagnosed as having, or suspected of having, one or more neurodegenerative diseases. In some embodiments, the subject comprises neuronal cells having a reduced concentration of free polynucleotides. Binding of the polynucleotide to the nucleic acid-binding polypeptide can treat and/or prevent neurodegenerative proteinopathies, and can further prevent cell death.

The administering step can include at least one, at least two, at least three, at least four, at least five, at least six, at least seven, at least eight, at least nine, or at least ten dosages. The administering step can be performed before the subject exhibits disease symptoms (e.g., prophylactically), or during or after disease symptoms occur. The administering step can be performed prior to, concurrent with, or subsequent to administration of other agents to the subject. In some embodiments, the administering step is performed prior to, concurrent with, or subsequent to the administration of one or more additional diagnostic or therapeutic agents. In some embodiments, the methods comprise administering one or more additional polynucleotides. In some embodiments, at least two, at least three, at least four, or at least five different polynucleotides are administered.

In some embodiments, a subsequent administration is provided at least one day after a prior administration, or at least two days, at least three days, at least four days, at least five days, or at least six days after a prior administration. In some embodiments, a subsequent administration is provided at least one week after a prior administration, or at least two weeks, at least three weeks, or at least four weeks after a prior administration. In some embodiments, a subsequent administration is provided at least one month, at least two months, at least three months, at least six months, or at least twelve months after a prior administration.

The amount of the disclosed compositions administered to a subject will vary from subject to subject, depending on the nature of the disclosed compositions and/or formulations, the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to reduce protein inclusions or to improve a symptom of a neurodegenerative disease). The dosage should not be so large as to outweigh benefits by causing extensive or severe adverse side effects, such as unwanted cross-reactions, anaphylactic reactions, and the like, although some adverse side effects may be expected. The dosage can be adjusted by the individual clinician in the event of any counterindications. Generally, the disclosed compositions and/or formulations are administered to the subject at a dosage of active component(s) ranging from 0.1 μg/kg body weight to 100 g/kg body weight. In some embodiments, the disclosed compositions and/or formulations are administered to the subject at a dosage of active component(s) ranging from 1 μg/kg to 10 g/kg, from 10 μg/kg to 1 g/kg, from 10 μg/kg to 500 mg/kg, from 10 μg/kg to 100 mg/kg, from 10 μg/kg to 10 mg/kg, from 10 μg/kg to 1 mg/kg, from 10 μg/kg to 500 μg/kg, or from 10 μg/kg to 100 μg/kg body weight. Dosages above or below the range cited above may be administered to the individual subject if desired. The compositions can be administered in any herein disclosed pharmaceutical composition comprising a pharmaceutically acceptable excipient.

In some embodiments, the subject comprises neuronal cells having a ratio of nucleic acid-binding polypeptide to free nucleic acids of at least 1:1, on a weight percent basis. In some embodiments, the subject comprises neuronal cells having a ratio of nucleic acid-binding polypeptide to free nucleic acids of from 1:1 to 100,000:1 or more, on a weight percent basis. In some embodiments, the subject comprises neuronal cells having a ratio of nucleic acid-binding polypeptide to free nucleic acids of at least 2:1, at least 3:1, at least 4:1, at least 5:1, at least 10:1, at least 50:1, at least 100:1, at least 500:1, at least 1,000:1, at least 10,000:1, or at least 100,000:1 on a weight percent basis.

### Methods of Inhibiting Aggregation

Also disclosed herein are methods to inhibit protein aggregation in a cell comprising contacting the cell with a composition comprising a polynucleotide that binds a nucleic acid-binding polypeptide, wherein the nucleic acid-binding polypeptide can aggregate in cells and is associated with a neurodegenerative disease. The nucleic acid-binding polypeptide can be any herein disclosed nucleic acid-binding polypeptide. The polynucleotide can be any herein disclosed polynucleotide, but can also include, in some embodiments, a naturally occurring polynucleotide, or a polynucleotide derived from a natural source (e.g., cleaved from a naturally occurring polynucleotide).

The cell can be any cell in which the nucleic acid-binding polypeptide can aggregate. In some embodiments, the cell is a cell of the nervous system (e.g., a glial cell or a neuronal cell). In some embodiments, the cell is a neuronal cell, optionally a human neuronal cell. In some embodiments, the cell is a muscular, connective, or epithelial cell. In some embodiments, the cell is a laboratory cell line (e.g., human embryonic kidney cells such as HEK293, HeLa cells, and osteosarcoma cells such as U2OS cells). The cell can be comprised within an isolated cell culture, or within a subject.

In some embodiments, the method reduces the rate of aggregation of the nucleic acid-binding polypeptide in the cell compared to an untreated control. In some embodiments, the method prevents aggregation of the nucleic acid-binding polypeptide in the cell compared to an untreated control.

The determined rates of aggregation can be compared to a control. In some embodiments, the rate of aggregation in the cell is at least 5% reduced compared to a control. In some embodiments, the rate or amount of aggregation in the cell is reduced by at least at least 10%, at least 25%, at least 30%, at least 40%, at least 50%, at least 60%, at least 70%, at least 80%, at least 90%, at least 95%, at least 98%, or at least 99% reduced compared to a control.

The control can comprise a biological sample, or alternatively, a collection of values used as a standard applied to one or more subjects (e.g., a general number or average that is known and not identified in the method using a sample). In some embodiments, the control can comprise an untreated biological sample of a subject having or suspected of having a neurodegenerative disease (e.g., a baseline sample). In some embodiments, the control can comprise untreated neuronal cells (e.g., from a subject having or suspected of having a neurodegenerative disease). In some embodiments, the control can be a treatment comprising a polynucleotide known not to bind to the nucleic acid-binding polypeptide (a non-targeting polynucleotide). For example, SEQ ID NO:120 is a non-targeting polynucleotide for TDP-43 because it is known not to bind to TDP-43. In some embodiments, the control can comprise a scrambled polynucleotide sequence.

In some embodiments, the cell comprises a ratio of nucleic acid-binding polypeptide (e.g., TDP-43, FUS, Tau) to free nucleic acids of at least 1:1, on a weight percent basis. In some embodiments, the cell comprises a ratio of nucleic acid-binding polypeptide to free nucleic acids of from 1:1 to 100,000:1 or more, on a weight percent basis. In some embodiments, the cell comprises a ratio of nucleic acid-binding polypeptide to free nucleic acids of at least 2:1, at least 3:1, at least 4:1, at least 5:1, at least 10:1, at least 50:1, at least 100:1, at least 500:1, at least 1,000:1, at least 10,000:1, or at least 100,000:1 on a weight percent basis.

In some embodiments, the methods comprise further administering a nuclease inhibitor, such as a RNase inhibitor, a DNase inhibitor, or combinations thereof. The nuclease inhibitor can be administered with the polynucleotide that binds a nucleic acid-binding polypeptide, wherein the nucleic acid-binding polypeptide can aggregate in cells and is associated with a neurodegenerative disease.

### Methods of Disaggregating Protein Aggregates

Also disclosed herein are methods to disaggregate protein aggregates in a cell comprising contacting the cell with a composition comprising a polynucleotide that binds a nucleic acid-binding polypeptide, wherein the nucleic acid-binding polypeptide has formed one or more aggregates in cells and is associated with a neurodegenerative disease. The nucleic acid-binding polypeptide can be any herein disclosed nucleic acid-binding polypeptide. The polynucleotide can be any herein disclosed polynucleotide, but can also include, in some embodiments, a naturally occurring polynucleotide, or a polynucleotide derived from a natural source (e.g., cleaved from a naturally occurring polynucleotide).

The cell can be any cell in which the nucleic acid-binding polypeptide has formed one or more aggregates (e.g., fibrils, inclusions) within the cell. In some embodiments, the cell is a cell of the nervous system (e.g., a glial cell or a neuronal cell). In some embodiments, the cell is a neuronal cell, optionally a human neuronal cell. In some embodiments, the cell is a muscular, connective, or epithelial cell. In some embodiments, the cell is a laboratory cell line (e.g., human embryonic kidney cells such as HEK293, HeLa cells, and osteosarcoma cells such as U2OS cells). The cell can be comprised within an isolated cell culture, or within a subject.

The method reduces the amount of pre-existing aggregates of the nucleic acid-binding polypeptide in the cell compared to an untreated control. In some further embodiments, the method also reduces the rate of further aggregation of the nucleic acid-binding polypeptide in the cell.

The determined amounts of protein aggregate can be compared to a control. In some embodiments, the amount of protein aggregate in the cell is at least 5% reduced compared to a control. In some embodiments, the amount of protein aggregate in the cell is reduced by at least 10%, at least 25%, at least 30%, at least 40%, at least 50%, at least 60%, at least 70%, at least 80%, at least 90%, at least 95%, at least 98%, or at least 99% reduced compared to a control.

The control can comprise a biological sample, or alternatively, a collection of values used as a standard applied to one or more subjects (e.g., a general number or average that is known and not identified in the method using a sample). In some embodiments, the control can comprise an untreated biological sample of a subject having or suspected of having a neurodegenerative disease (e.g., a baseline sample). In some embodiments, the control can comprise untreated neuronal cells (e.g., from a subject having or suspected of having a neurodegenerative disease). In some embodiments, the control can be a treatment comprising a polynucleotide known not to bind to the nucleic acid-binding polypeptide (a non-targeting polynucleotide). In some embodiments, the control can comprise a scrambled polynucleotide sequence.

In some embodiments, the cell comprises a ratio of nucleic acid-binding polypeptide (e.g., TDP-43, FUS, Tau) to free nucleic acids of at least 1:1, on a weight percent basis. In some embodiments, the cell comprises a ratio of nucleic acid-binding polypeptide to free nucleic acids of from 1:1 to 100,000:1 or more, on a weight percent basis. In some embodiments, the cell comprises a ratio of nucleic acid-binding polypeptide to free nucleic acids of at least 2:1, at least 3:1, at least 4:1, at least 5:1, at least 10:1, at least 50:1, at least 100:1, at least 500:1, at least 1,000:1, at least 10,000:1, or at least 100,000:1 on a weight percent basis.

In some embodiments, the methods comprise further administering a nuclease inhibitor, such as a RNase inhibitor, a DNase inhibitor, or combinations thereof. The nuclease inhibitor can be administered with the polynucleotide that binds a nucleic acid-binding polypeptide, wherein the nucleic acid-binding polypeptide has formed aggregates in cells and is associated with a neurodegenerative disease.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. RNA Binding Antagonizes Neurotoxic Phase Transitions of TDP-43

A novel optogenetic-based method to reliably induce TDP-43 proteinopathy with light was developed. The method was used to examine mechanisms that drive the formation of intracellular inclusions. The method demonstrated that light-induced phase separation (LIPS) seeds pathologically-relevant and neurotoxic TDP-43 inclusions. The pathobiology underlying this process was revealed and it was shown that aberrant homo-oligomerization of the TDP-43 LCD mediates intracellular phase separation interactions that can mature into pathological inclusions. Remarkably, the RNA-binding status of TDP-43 drives its homo-oligomerization and RNA treatment prevents TDP-43 aggregation. TDP-43 targeting to SGs, which contain high concentrations of RNA, requires RNA-binding and, in surprising contrast to existing theories, this recruitment prevents the formation of pathological TDP-43 inclusions.

### Results

### Optogenetic Modulation of TDP-43 Inclusions.

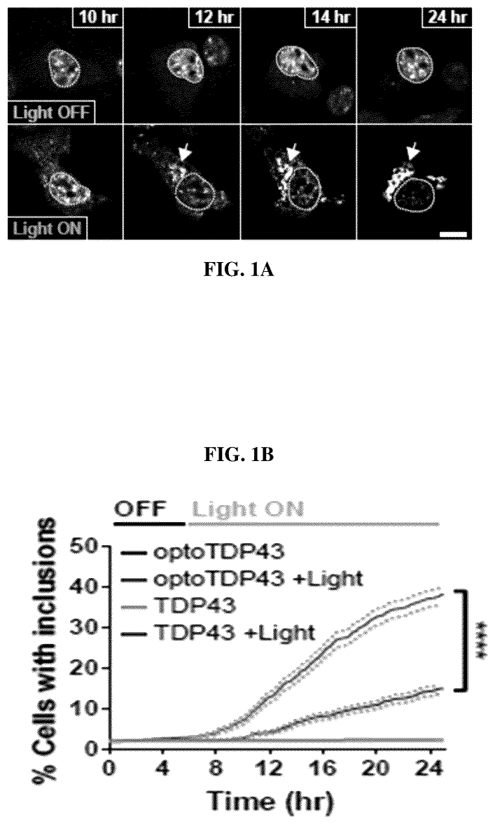

An optogenetic Cry2olig-TDP-43-mCherry expression construct (optoTDP43) was generated to selectively induce TDP-43 proteinopathy under the spatiotemporal control of light stimulation. Cry2olig is a variant of the Photolyase-Homologous Region (PHR) of the Cryptochrome 2 protein from Arabidopsis thaliana that undergoes reversible homo-oligomerization (in about 5 min) in response to blue light (Taslimi, et al., (2014), Nat. Commun. 5, 4925). N-terminal oligomerization is important in promoting higher-order assembly and LLPS of TDP-43 (Afroz, et al. (2017), Nat. Commun. 8, 45; Chang, et al., (2012), Biochem. Biophys. Res. Commun. 425, 219-224; Mompean, et al., (2017), J. Biol. Chem. 292, 11992-12006; Wang, et al., (2018), EMBO J. 37). Thus, the Cry2olig domain was positioned on the N-terminus of the full-length TDP-43 protein. It was first tested whether Cry2olig-mediated increases in focal concentrations of optoTDP43 protein leads to intracellular TDP-43 proteinopathy upon light exposure. To monitor this event, automated epifluorescence imaging was performed on live HEK293 cells expressing the mCherry-tagged optoTDP43 protein. Cells were intermittently imaged during a persistent blue light treatment using a 96-well LED array positioned within a stage-top incubator. optoTDP43-expressing cells exposed to blue light showed a progressive depletion of nuclear optoTDP43 signal and developed significantly more cytoplasmic inclusions relative to optoTDP43-expressing cells kept in the dark over a 24 hr live-imaging session (FIG. TA and FIG. 1B). TDP-43 was not mislocalized in cells expressing Cry2olig-mCh alone with or without light exposure, as assessed by immunofluorescence and subcellular fractionation/western blotting. TDP-43 inclusions were absent from TDP-43-mCh-expressing cells with or without light exposure during the imaging period indicating that optoTDP43 inclusion formation is selectively driven by light-induced oligomerization of TDP-43 and not TDP-43 overexpression (FIG. 1B). Consistent with this observation, size-exclusion chromatography confirmed that light treatment induced the formation of higher-order optoTDP43 protein oligomers. Together, these data show that light treatment initiates intracellular oligomerization of the optoTDP43 protein independent of overexpression-induced aggregation.

It was next evaluated whether the features of optoTDP43 inclusions recapitulate the biochemical and neuropathological hallmarks of TDP-43 proteinopathy seen in patient tissue, including detergent-insolubility, N-terminal cleavage, C-terminal hyperphosphorylation, p62 co-localization, and the formation of ubiquitin-enriched inclusions (Al-Sarraj, et al., (2011), Acta Neuropathol. 122, 691-702; Scotter, et al., (2015), Neurotherapeutics 12, 352-363). Fluorescence recovery after photo-bleaching (FRAP) analysis of light-induced optoTDP43 inclusions was performed to first assess the material state of light-induced optoTDP43 inclusions. While the Cry2-mCh signal recovered immediately, the optoTDP43 inclusions exhibited minimal signal recovery following photobleaching, showing the formation of immobile intracellular protein assemblies. In addition, detergent-solubility fractionation and immunoblotting for TDP-43 and mCherry proteins showed that light induced a dramatic shift in optoTDP43 to the insoluble fraction and promoted N-terminal cleavage, mimicking the biochemical profile observed in ALS/FTLD patient tissue. TDP-43-mCh-expressing cells did not display a similar shift in solubility or cleavage in the presence or absence of light when examined at the same timepoint. Endogenous TDP-43 was also detected in the insoluble fraction and exhibited enhanced N-terminal cleavage in optoTDP43-expressing cells exposed to light. Size-exclusion chromatography similarly revealed a shift of endogenous TDP-43 species to heavier fractions relative to cells in the dark. These combined data show that optoTDP43 sequesters non-optogenetic TDP-43 proteins into light-induced insoluble fraction. This phenomenon is likely due to TDP-43-specific interactions, as Cry2olig-mCh expression did not affect endogenous TDP-43 solubility or cleavage regardless of light exposure.

The ability of optoTDP43 to directly recruit other non-optogenetic TDP-43 species into light-induced inclusions was next confirmed by co-expressing an EGFP-tagged TDP-43 construct along with optoTDP43 or Cry2-mCh alone. It was found that EGFP-TDP-43 co-localized with the light activated optoTDP43 inclusions, while no such co-localization was observed with Cry2-mCh. Furthermore, optoTDP43 inclusions are p62-positive (82.9%), hyperphosphorylated in the C-terminal domain (85.7%), and colocalized with ubiquitin, as seen in patient tissue. Together, these data show that light-activated optoTDP43 inclusions exhibited the biochemical and pathological hallmarks of TDP-43 proteinopathy and could seed endogenous TDP-43 aggregation.

### Aberrant LCD Phase Transitions Drives the Formation of TDP-43 Inclusions.

Studies indicate that low complexity domains (LCDs) mediate the homo- and hetero-oligomerization of LCD-containing proteins through liquid-liquid phase separation (LLPS) (Elbaum-Garfinkle, et al., (2015), Proc. Natl. Acad. Sci. USA 112, 7189-7194; Molliex, et al., (2015), Cell 163, 123-133; Nott, et al., (2015), Mol. Cell 57, 936-947) and aberrant interactions between these low-complexity regions are hypothesized to promote the maturation/fibrillization of phase-separated droplets (Harrison, et al., (2017), Biochem. J. 474, 1417-1438). To determine whether the TDP-43 LCD mediates the formation of optoTDP43 inclusions, the optoDroplet approach (Shin, et al., (2017), Cell 168, 159-171.e14) was employed to study intracellular TDP-43 LCD phase separation behavior using the WT Cry2 PHR domain (Cry2 PHR). This photoreceptor has a higher saturation concentration as compared to the lower-threshold, and more potent, Cry2olig photoreceptor used to produce optoTDP43 inclusions, and facilitates detection of subtle changes in droplet properties. An optogenetic construct was generated containing the Cry2 PHR photoreceptor fused to the TDP-43 LCD (optoLCD). optoLCD proteins readily underwent reversible light-induced phase separation (LIPS) in response to brief pulses of blue light in HEK293 cells. These findings are consistent with studies reporting that purified TDP-43 LCD protein undergoes LLPS in vitro (Conicella, et al., (2016), Structure 24, 1537-1549; Sun, et al., (2017), Biochemistry 56, 809-823). Light-induced clustering of the Cry2 PHR photoreceptor alone did not induce droplet formation, showing that the TDP-43 LCD drives LIPS. Light-activated optoLCD granules also displayed classical properties of phase-separated liquid-like protein droplets (Brangwynne, et al., (2009), Science 324, 1729-1732; Brangwynne, et al., (2015), Nat. Phys. 11, 899-904), undergoing fusion upon granule contact and exhibiting concentration- (wherein a plot of fluorescence vs. number of granules per cell had a concentration-dependent r² of 0.902) and light-dependent droplet formation. These data are consistent with light-activated optoDroplet formation of the LCDs of other RNA-binding proteins, including FUS, HNRNPA1 and DDX4 (Shin, et al., (2017), Cell 168, 159-171.e14).

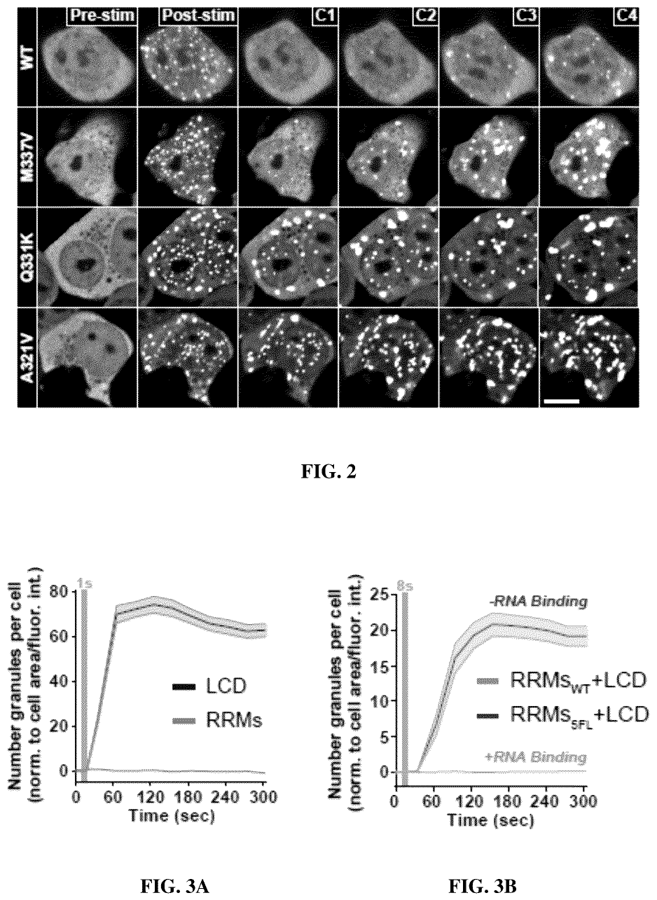

To determine whether aberrant LCD interactions promote the formation of intracellular inclusions, the effects of repetitive and chronic LIPS on optoLCD droplet dynamics were examined in live cells. The examined proteins included wildtype TDP-43 LCD and optoLCD proteins containing three ALS-causing mutations (M337V, Q331K, A321V), which alter LLPS properties and promote aggregation of TDP-43 in diverse in vitro and cellular models (Gopal, et al., (2017), Proc. Natl. Acad. Sci. USA 114, E2466-E2475; Johnson, et al., (2009), J. Biol. Chem. 284, 20329-20339; Schmidt, et al., (2016), Cell Rep. 16, 1228-1236). Using a 10-minute cycling blue light stimulation protocol to induce repetitive LIPS of the LCD, gradual maturation of optoLCD granules was observed over successive cycles of droplet formation (FIG. 2). optoLCD droplets exhibited a progressive increase in the number of persistent, irreversible optoLCD granules with each successive cycle, an effect that was exacerbated by ALS-linked mutations (FIG. 2). Mutant optoLCD proteins displayed more rapid conversion to stable droplets upon successive LIPS, as determined by an increased number and size of persistent granules, compared to WT optoLCD droplets despite similar protein level as determined by Western blot analysis. A chronic blue light stimulation paradigm was then used to investigate whether the WT optoLCD droplets matured into pathologically-relevant inclusions with prolonged LCD oligomerization. Interestingly, a 4 hr chronic blue light stimulation resulted in hyperphosphorylated and p62-positive optoLCD inclusions that were immobile as evaluated by quantitative FRAP analysis. These results show that TDP-43 LCD undergoes intracellular phase separation upon self-oligomerization and that maturation of LCD droplets, either through repetitive/chronic phase transitions or ALS-linked mutations, drives LCD inclusion formation.

### RNA-Binding Inhibits TDP-43 LCD Homo-Oligomerization.

While the TDP-43 LCD underwent intracellular LIPS with acute blue light treatment, the same light stimulation paradigm did not induce a phase transition of full-length optoTDP43, even when fused to the more potent homo-oligomerizing Cry2 photoreceptor domain (Cry2olig) (Taslimi, et al., (2014), Nat. Commun. 5, 4925). Given the ability of full-length purified TDP-43 to undergo rapid LLPS (McGurk, et al., (2018), Mol. Cell 71, 703-717.e9; Molliex, et al., (2015), Cell 163, 123-133; Wang, et al., (2018), EMBO J. 37), it was suspected there may be components within the intracellular environment which oppose TDP-43 LLPS in the model. In view of the presence of the prominent TDP-43 RNA-binding domains (or RNA-recognition motifs, RRMs), and along with reports of an increased aggregation propensity of C-terminal cleavage products lacking these domains (Zhang, et al., (2009), Proc. Natl. Acad. Sci. USA 106, 7607-7612), it was examined whether RNA-binding inhibits the ability of the full-length optoTDP43 protein to homo-oligomerize via its LCD.

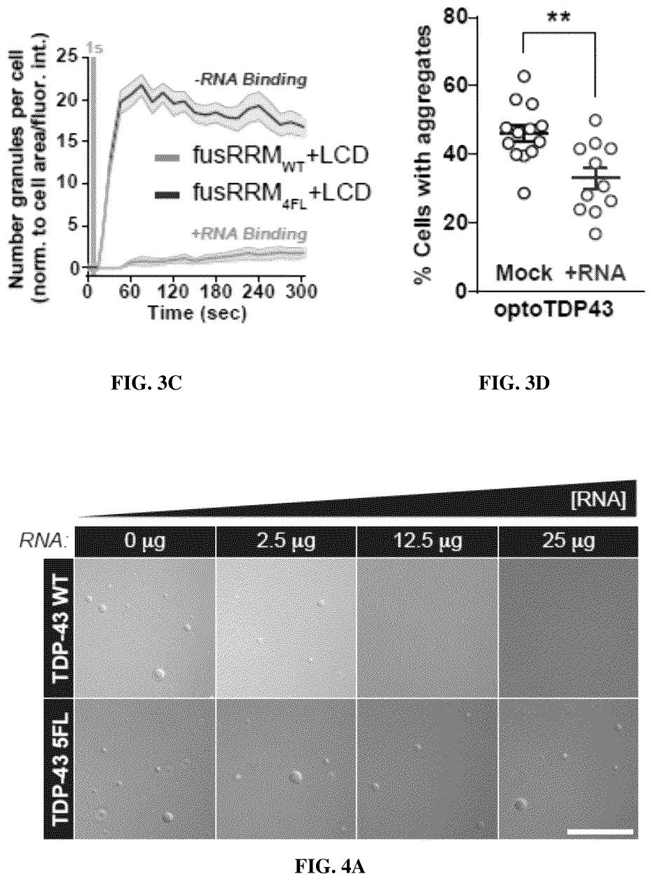

To test this notion, the Cry2olig photoreceptor domain was fused to the TDP-43 RRMs and the ability of the RRM domains to oligomerize in response to light was examined. The enhanced homo-oligomerization of the Cry2olig photoreceptor domain helped ensure that any observed inhibition of LIPS was likely a result of endogenous TDP-43 properties and not due to insufficient Cry2 activation. While the TDP-43 LCD fused to Cry2olig domain (Cry2olig-LCD) underwent LIPS in response to blue light and mimicked the droplet-like properties seen with optoLCD assemblies, light treatment did not stimulate TDP-43 RRM phase separation (FIG. 3A). Next, fusion proteins were generated containing both the TDP-43 RRMs and Cry2olig-LCD (RRMs+LCD WT) to test whether the RRM domains affect TDP-43 LCD LIPS. Strikingly, the RRMs completely inhibited LIPS of the LCD, even when the potent Cry2olig photoreceptor was employed (FIG. 3B). To test whether RNA binding to the RRMs inhibits TDP-43 LCD oligomerization, five point phenylalanine to leucine mutations were introduced within the RRM domains (5FL), which were previously shown to significantly impair, but not abolish, TDP-43 RNA-binding (RRMs+LCD 5FL) (Elden, et al., (2010), Nature 466, 1069-1075). Remarkably, robust induction of LIPS was observed following light stimulation (FIG. 3B). Since Cry2olig fusion proteins containing mutated RRMs alone (RRMs 5FL) did not show any light-induced droplet formation, recovery of LIPS in the RNA-binding deficient construct was mediated by the LCD. To ensure that RNA-binding activity, and not a conformational change in the construct, inhibited LIPS, it was assessed whether the TDP-43 LCD could drive phase separation of the FUS RNA-binding protein RRM (fusRRM WT+LCD). Like the TDP-43 RRMs, the FUS RRM fusion to the TDP-43 LCD failed to undergo LIPS (FIG. 3C). However, when similar phenylalanine to leucine point mutations that compromise the RNA-binding ability of the FUS RRM were introduced (Daigle, et al., (2013), Hum. Mol. Genet. 22, 1193-1205) (fusRRM 4FL+LCD), the FUS RRM+TDP-43 LCD fusion protein underwent rapid LIPS (FIG. 3C). These data show that RNA-binding dictates the ability of the TDP-43 LCD to homo-oligomerize and drive LIPS.

To determine whether RNA-binding also plays a role in the formation of pathological inclusions of full-length TDP-43, RNA fluorescent in situ hybridization (FISH) was performed using an RNA dye to assess whether RNA resides within the light-induced optoTDP43 inclusions. Interestingly, RNA was absent in light-induced optoTDP43 inclusions. Supporting the notion that RNA-binding dictates the ability of the LCD to drive pathological oligomerization of TDP-43, longitudinal imaging also revealed that RNA-deficient optoTDP43 5FL formed inclusions at a significantly enhanced rate and frequency than the RNA-binding competent optoTDP43 in live cells.

Since the obtained data showed that RNA-binding inhibits LCD-driven phase-transitions of TDP-43, it was tested whether addition of exogenous RNA substrates inhibit optoTDP43 inclusion formation. optoTDP43-expressing HEK293 cells were treated with blue light for 16 hr to induce inclusions and transfected cells with total HEK293 cell RNA 4 hr after illumination. Surprisingly, there was a measurable reduction (28%) of optoTDP43 inclusion formation in cells treated with purified RNA, as compared to mock-treated cells, following light stimulation (FIG. 3I). To determine whether mRNA colocalized with TDP-43 inclusions in patients, RNA FISH was performed using a poly-T probe in ALS/FTLD patient tissue and TDP-43/mRNA colocalization was examined. TDP-43 inclusions lacked mRNA signal in both sporadic ALS (sALS) spinal cord and FTLD hippocampus. Taken together, RNA-binding to the TDP-43 RRM domains inhibited LCD:LCD interactions, thus blocking aberrant TDP-43 phase transitions and the formation of pathological inclusions.

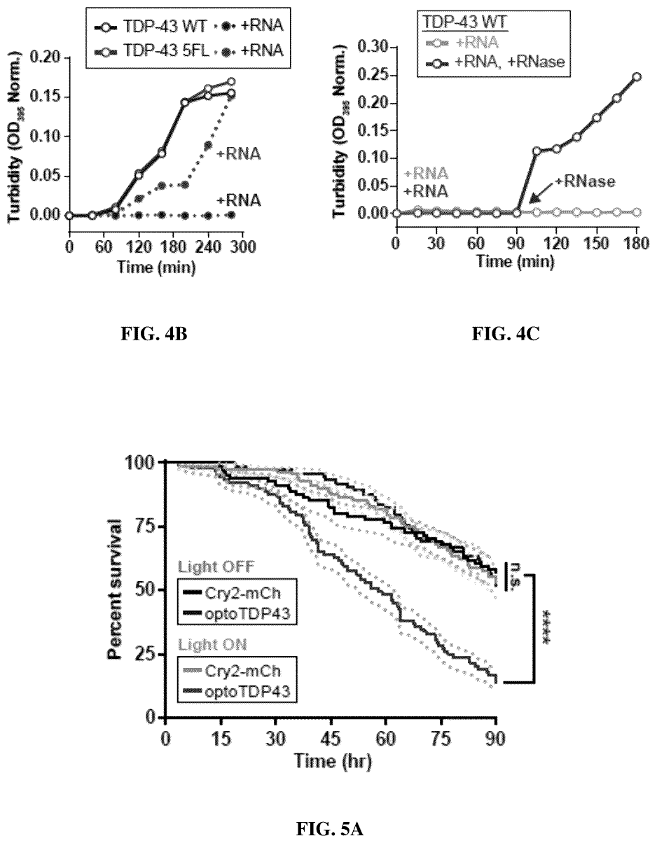

Next, purified WT TDP-43 and a TDP-43 protein with identical RRM point mutations (TDP-43 5FL, henceforth referred to as RNA-binding deficient TDP-43) were used to examine whether RNA-binding altered TDP-43 LLPS and aggregation in vitro. In the absence of RNA, C-terminal MBP-tagged TDP-43 WT and 5FL formed liquid-like droplets at physiological salt concentrations that did not form in the presence of 1,6 hexanediol, which disrupts weak contacts between LCDs that drive LLPS (Gopal, et al., (2017), Proc. Natl. Acad. Sci. USA 114, E2466-E2475; Patel, et al., (2007), Cell 129, 83-96). However, in the presence of increasing concentrations of total RNA, WT TDP-43 LLPS was inhibited in a dose-dependent manner (FIG. 4A). In contrast, the RNA-binding deficient TDP-43 5F1L species was unaffected by exogenous RNA (FIG. 4A). Upon selective cleavage of the MBP tag with TEV protease, TDP-43 WT and 5FL formed solid-phase aggregates. While WT and RNA-binding-deficient TDP-43 displayed similar aggregation kinetics in the absence of RNA, the addition of RNA completely inhibited WT TDP-43 aggregation (FIG. 4B). The aggregation kinetics of TDP-43 5FL was initially delayed in the presence of RNA, likely due to residual RNA-binding (FIG. 4B). However, the final extent of aggregation was identical to TDP-43 in the absence of RNA upon further incubation (FIG. 4B). Moreover, the addition of RNase A (2.5 μg) 90 min after TEV cleavage reversed the initial inhibition of WT TDP-43 aggregation conferred by RNA addition (FIG. 4C).

If RNA-binding prevented aberrant phase separation and TDP-43 inclusion formation, then RNA-binding deficient protein expression should generate TDP-43 proteinopathy. Thus, non-optogenetic EGFP-tagged TDP-43 constructs containing wildtype or RNA-binding-reduced (5FL) RRM domains were expressed in HEK293 cells. Mere expression of the RNA-binding-deficient TDP-43 protein (EGFP-TDP43 5FL) generated large nuclear inclusions that were hyperphosphorylated and p62-positive. When the protein's NLS was mutated (EGFP-TDP43cyto 5FL), the RNA-binding-deficient protein localized to the cytoplasm and formed hyperphosphorylated and p62-positive inclusions that resembled the neuropathology observed in ALS/FTLD. This finding showed that cytoplasmic mislocalization is an upstream event preceding formation of RNA-deficient cytoplasmic inclusions. FRAP analysis revealed that RNA-deficient TDP-43 inclusions in both the nucleus and cytoplasm did not recover from photo-bleaching. Nuclear RNA-binding deficient TDP-43 inclusions also exhibited slightly enhanced fluorescence recovery after photo-bleaching compared to the cytoplasmic RNA-deficient TDP-43. This may be due to the abundance of accessible RNA species in the nucleus whereas RNAs destined for the cytoplasm are pre-assembled into heterogenous RNP complexes (K6hler, et al., (2007), Nat. Rev. Mol. Cell Biol. 8, 761-773). These findings show that RNA-binding dictates the propensity for TDP-43 to form pathologically-relevant inclusions.

### Impaired SG Recruitment Promotes Aberrant TDP-43 Phase Transitions.

SG components are modifiers of toxicity and pathological aggregation associated with TDP-43-overexpression in yeast, fly, and mouse models (Becker, et al., (2017), Nature 544, 367-371; Elden, et al., (2010), Nature 466, 1069-1075; Kim, et al., (2014), Nat. Genet. 46, 152-160). SGs are composed of high concentrations of LCD-containing proteins and form through LLPS (Shin, et al., (2017), Science 357), and maturation of TDP-43-containing SGs may seed pathological inclusions (Harrison, et al., (2017), Biochem. J. 474, 1417-1438). Since RNA-binding dictates the ability of TDP-43 to undergo LLPS and inclusion formation, it was determined whether reducing the RNA-binding ability of TDP-43 would confer enhanced SG recruitment and subsequent maturation into insoluble inclusions. Because cytoplasmic mislocalization is required for TDP-43 recruitment to SGs (Bentmann, et al., (2012), J. Biol. Chem. 287, 23079-23094), SG formation was first induced in cells expressing cytoplasmic TDP-43 (EGFP-TDP43cyto) with functional (WT) or RNA-binding deficient (5FL) RRMs. Co-localization with known SG markers was examined. Notably, RNA-binding-deficient TDP-43 (TDP43cyto 5FL) was essentially completely excluded from endogenous SGs upon heat shock or sodium arsenite-induced cell stress, whereas about 70% of RNA-binding-competent TDP-43 was recruited to SGs under both stressors. However, during this analysis a subset (about 30%) of cytoplasmic WT TDP-43 granules was found which formed under stress conditions and did not co-localize with SG markers. Further investigation revealed that these TDP-43 granules (SG− granules) lacked mRNA and were enlarged by about 3-4 fold mean granule area. In contrast, cytoplasmic TDP-43 recruited to SGs (SG+) co-localized with mRNA signal. FRAP analysis of these two subsets of cytoplasmic TDP-43 granules in live cells similarly revealed that TDP-43 recruited to RNA-containing stress granules (SG+) remained dynamic, whereas the excluded TDP-43 species (SG−) were static. Furthermore, TDP-43 residing in SG− inclusions were hyperphosphorylated and p62-positive, thus exhibiting pathological hallmarks of TDP-43 proteinopathy. This result shows that TDP-43 recruitment to SGs required RNA binding and that localization to acutely-formed, RNA-rich SGs promoted TDP-43 solubility, whereas TDP-43 outside of these RNA-containing assemblies were insoluble.

In support, TDP-43 5FL inclusions did not co-localize with SG proteins in the nucleus or in the cytoplasm in HEK293 cells, likely due to RNA-binding deficiencies. Furthermore, although optoTDP43 proteins were recruited to SGs upon sodium arsenite treatment, optoTDP43 inclusions of all sizes were devoid of key stress granule components (Boeynaems, et al., (2016), Acta Neuropathol. 132, 159-173) after illumination. Neuropathological analysis of ALS/FTLD patient tissue similarly revealed an absence of co-localization between TDP-43 inclusions and G3BP1/ATXN2.

### Bait Oligonucleotides Rescue Aberrant TDP-43 Phase Transitions and Neurotoxicity

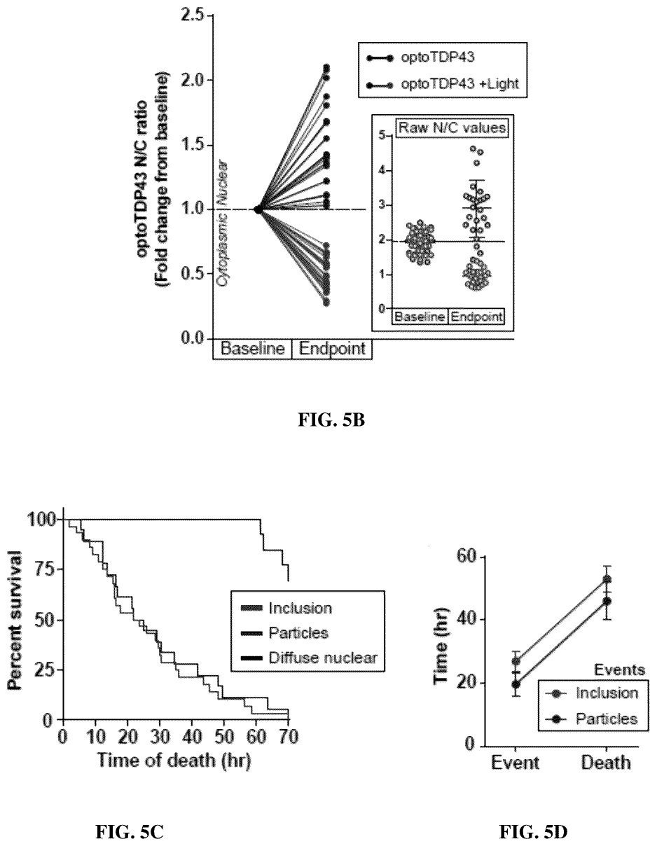

TDP-43 proteinopathy correlates with regions of neurodegeneration in ALS/FTD patients (Mackenzie, et al., (2013), Acta Neuropathol. 126, 859-879). To determine whether the formation of TDP-43 inclusions is toxic to human neurons, human ReNcell VM neural progenitor cells were differentiated into cortical-like neurons and obtained highly enriched neuronal cultures (Donato, et al., (2007), BMC Neurosci. 8, 36). A lentiviral expression system was used to selectively express optoTDP43 and a far-red fluorescent reporter (iRFP670) in human ReNcell cortical neuron cultures under the control of the human synapsin promoter (hSyn) prior to induction of optoTDP43 inclusion formation with chronic light stimulation. Longitudinal live-imaging at 30 min intervals revealed no significant neuronal loss in optoTDP43-expressing neurons kept in the dark (FIG. 5A). However, optoTDP43 neurons exposed to blue light exhibited progressive blebbing and loss of iRFP670 fluorescent signal in the soma after about 28 hr of light exposure (determined by Chi-Square analysis) (FIG. 5A). At the final 90 hr timepoint, a 4.3-fold increase in cell death in optoTDP43-expressing neurons exposed to light was observed in comparison to those maintained in darkness (FIG. 5A). No significant decrease in cell survival was recorded in neurons expressing the Cry2-mCh photoreceptor alone regardless of light treatment (FIG. 5A). Light stimulation alone also failed to induce alterations in neuron morphology or cell viability, indicating no phototoxicity due to the light exposure. Subsequent analyses revealed a striking cytoplasmic shift of optoTDP43 signal in dying cells about 1 hr before cell death (FIG. 5B) that was followed by formation of either large optoTDP43 inclusions or smaller, more circular assemblies (particles). No significant differences were observed in either the overall survival patterns (FIG. 5C) or the time-to-death following detection of either of these assembly types in individual neurons (Event-Death) (FIG. 5D). Importantly, neurons harboring either optoTDP43 assemblies exhibited reduced survival as compared to those with diffuse nuclear signal throughout the imaging period (FIG. 5C). Immunofluorescence analysis of neurons at the 48 hr timepoint confirmed the presence of hyperphosphorylated and p62-positive optoTDP43 inclusions in the cytoplasm of ReNcell neurons exposed to light. However, examination of the smaller and equally toxic optoTDP43 particles revealed no co-localization with these markers. These observations show that aberrant cytoplasmic phase transitions drive neurodegeneration and exert toxic downstream effects independent of 5409/5410 phosphorylation or p62-colocalization status.

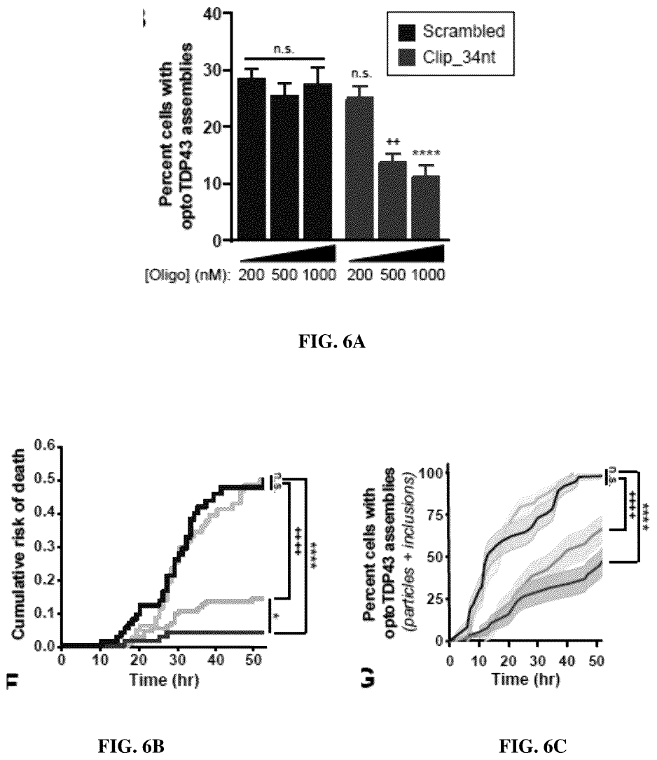

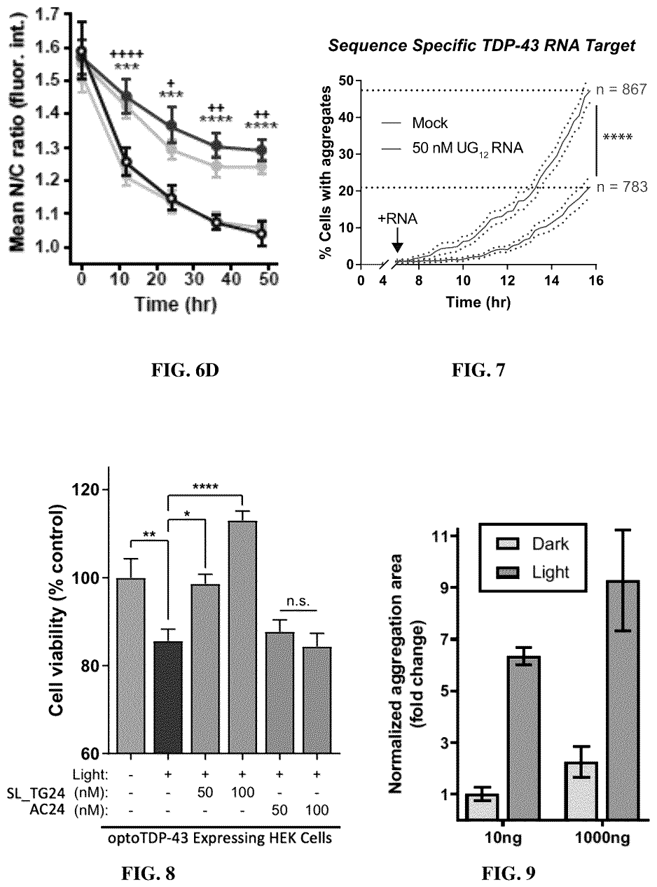

To investigate whether the neurotoxicity associated with aberrant optoTDP43 phase transitions could be mitigated through enhanced RNA binding, a 2′OMe-modified RNA oligonucleotide was designed based on a well-characterized TDP-43 binding sequence (Clip_34nt; SEQ ID NO:13) previously shown to exhibit a high affinity for TDP-43 RRMs and a Kd of 112 nM (Ayala, et al., (2011), EMBO J. 30, 277-288; Bhardwaj, et al., (2013), 41, 5062-5074). The Clip_34nt was used to assess the ability of these bait oligonucleotides (bONs) to prevent light-induced optoTDP43 phase transitions in HEK293 cells. Cells expressing optoTDP43 were treated with either Clip_34nt or a scrambled oligonucleotide of equal length and nucleic acid composition prior to an 8 hr light stimulation. Similar to the effect produced by total HEK293 RNA, treatment with the bONs resulted in a dose-dependent reduction in cytoplasmic optoTDP43 assemblies (FIG. 6A), in contrast to the non-targeting scrambled control. To test whether preventing light-induced optoTDP43 phase transitions enhanced neuronal survival, automated longitudinal imaging of optoTDP43-expressing ReNcell cortical neurons was performed following a 4 hr pre-treatment with either Clip_34nt 20 or the control oligonucleotide. optoTDP43 expression was induced for 96 hrs, then the neurons were co-incubated with RNAs for 4 hrs, after which time automated imaging in the presence of blue light occurred over the next 50 hrs. While no significant differences were observed in cumulative risk-of-death, a significant and dose-dependent reduction in neurotoxicity during light treatment following treatment with the bONs was observed (FIG. 6B). Decreased toxicity was associated with similar dose-dependent reductions in light-induced optoTDP43 phase transitions, both inclusions and particles (FIG. 6C), and nuclear-cytoplasmic redistribution of the optoTDP43 protein (FIG. 6D). Consistent with results presented above, these studies show that the neuroprotective effect of a TDP-43-binding oligonucleotide occurs through the prevention of aberrant and neurotoxic cytoplasmic TDP-43 phase transitions.

### DISCUSSION

TDP-43 proteinopathy is a pathological hallmark in several neurodegenerative disorders. Modeling this pathology has proven challenging and no standard currently exists to reliably reproduce TDP-43 inclusions in live cells. To address this problem, a photokinetic system was developed to selectively induce TDP-43 proteinopathy that recapitulates pathological features observed in ALS/FTD and other neurodegenerative diseases. This system facilitated the study of intracellular mechanisms driving pathological phase separation. The intracellular phase transitions of TDP-43 are mediated by the LCD, and events which promote homo-oligomerization of the TDP-43 LCD seed neuropathological inclusions. Surprisingly, RNA-binding status dictates TDP-43 LCD-mediated oligomerization and RNA treatment reduces LLPS, aggregation of purified TDP-43, and light-induced optoTDP43 inclusion formation in live cells. Given recent evidence suggesting a role for SGs in seeding TDP-43 inclusions (Fernandes, et al., (2018), Adv. Neurobiol. 20, 173-212), it was hypothesized that the heightened ability of RNA-binding deficient TDP-43 to undergo LLPS and aggregation confers enhanced recruitment to phase-separated SGs. Surprisingly, RNA-binding deficient TDP-43 is excluded from acutely-induced SGs and instead found in inclusions devoid of SG components. In contrast, TDP-43 recruited to SGs remained dynamic within these RNA-rich, liquid-like compartments. Since RNA binding inhibits aberrant phase transition of TDP-43, it was tested whether a TDP-43-binding oligonucleotide sequence inhibited a pathogenic event. Remarkably, treatment with the oligonucleotide mitigated the cytoplasmic mislocalization and aberrant phase transition of optoTDP43 in response to light and rescued neurotoxicity.

Therefore, RNA-binding dictates the ability of optoTDP43 to form light-induced inclusions. LCD oligomerization of RNA-bound optoTDP-43 is blocked upon Cry2-mediated increases in local protein concentration while RNA-binding deficient optoTDP43 LCDs associate, thereby promoting inclusion formation. This phenomenon could arise from a conformational change associated with nucleic acid binding, as shown for other RNA-binding proteins (Williamson, J.R. (2000), Nat. Struct. Biol. 7, 834-837). Nucleic acid binding also maintains the TDP-43 dimer and promotes solubility, potentially through an allosteric prevention of LCD:LCD interactions (Afroz, et al. (2017), Nat. Commun. 8, 45; Sun, et al., (2014), Biochemistry 53, 5885-5894). It is also possible that RNA-binding impedes TDP-43 homo-oligomerization through competitive inhibition. While the LCD appears to be dispensable for RNA binding (Buratti, et al., (2001), J. Biol. Chem. 276, 36337-36343), it contains an RGG motif that can mediate RNA interactions (Conicella, et al., (2016), Structure 24, 1537-1549). Consistent with this, RNA depletion results in the in vitro oligomerization and aggregation of a C-terminal TDP-43 cleavage product lacking RRM1 and a portion of RRM2 (TDP-25) (Kitamura, et al., (2016), Sci. Rep. 6, 19230). Contacts within the TDP-43 RRMs themselves may also cooperate and play a distinct role in the TDP-43 aggregation process, as both RRM1 and RRM2 can form tetrameric assemblies in vitro (Kuo, et al., (2009), Nucleic Acids Res. 37, 1799-1808), and the RRM2 contains various segments capable of forming amyloid-like conformations (Guenther, et al., (2018), Nat. Struct. Mol. Biol. 25, 311-319). Overall, interactions between these domains and RNA may block regions that mediate aberrant TDP-43 phase transitions. Similarly, FUS protein LLPS is inhibited by the nuclear import receptor Karyopherin-P2 due to competitive interactions within domains that drive self-association (Guo, et al., (2018), Cell 173, 677-692.e20; Yoshizawa, et al., (2018), Cell 173, 693-705.e22).

This work shows that an altered TDP-43:RNA ratio creates an aggregation-prone environment for TDP-43 and serves as an upstream event in TDP-43 proteinopathy. This may occur due to disease causing mutations such as C9orf72 ALS/FTD that disrupt nuclear transport dynamics resulting in elevated cytoplasmic TDP-43 protein (Zhang, et al., (2015b), Nature 525, 56-61) and retention of RNA in the nucleus (Freibaum, et al., (2015), Nature 525, 129-133). Disease-causing mutations in the TDP-43 RRMs that abolish RNA binding have not yet been described. However, disease-associated mutations within the TDP-43 LCD likely promote aberrant phase transitions of RNA-deficient TDP-43 proteins. This RNA-dependent model explains the aggregation of TDP-43 observed in overexpression models, in which insoluble inclusions may result from concentration-dependent phase transitions of cytoplasmic TDP-43 due to a lack of RNA substrates, as well as the cytoplasmic localization of TDP-43 inclusions observed in patient tissue (Scotter, et al., (2015), Neurotherapeutics 12, 352-363). Furthermore, this phenomenon likely serves as the convergence point for a number of pathways proposed to contribute to ALS/FTD including altered TDP-43 expression, half-life and clearance; disrupted nucleocytoplasmic transport; and abnormal RNP assembly and trafficking (Boeynaems, et al., (2016), Acta Neuropathol. 132, 159-173; Gopal, et al., (2017), Proc. Natl. Acad. Sci. USA 114, E2466-E2475; Ling, et al., (2013), Neuron 79, 416-438).

SG-mediated seeding likely underlies the formation of TDP-43 inclusions. For example, the modulation of SG components alleviates TDP-43 inclusion formation and toxicity in overexpression models (Becker, et al., (2017), Nature 544, 367-371; Elden, et al., (2010), Nature 466, 1069-1075; Kim, et al., (2014), Nat. Genet. 46, 152-160). While previous studies show that both the TDP-43 RRMs and LCD are necessary for SG targeting (Colombrita, et al., (2009), J. Neurochem. 111, 1051-1061), it is shown herein that TDP-43 RNA binding facilitates SG localization. Furthermore, it was observed that cytoplasmic TDP-43 excluded from acute SGs forms pathological inclusions lacking mRNA, whereas TDP-43 recruited to SGs is dynamic and mobile. This is likely due to the abundance of RNA and other LCD-containing RBPs within SGs. These dynamic protein:protein, protein:RNA, and RNA:RNA interactions within SGs likely contribute to the liquid-like properties of these structures and may convey a diminished propensity to initiate pathological maturation/fibrillization as compared to a more homogenous, protein-rich granule. Therefore, these findings delineate between normal, physiological LLPS interactions, specifically in the formation of heterogeneously-composed membraneless organelles, and pathological homogeneous LLPS interactions prone to seed disease-associated protein inclusions.

It is unclear whether aberrant phase transitions or inclusions themselves are inherently neurotoxic and/or capable of driving neurodegeneration. ALS-linked mutations in TDP-43 that increase aggregation propensity result in enhanced toxicity in a variety of in vitro and in vivo models (Janssens, et al., (2013), Hum. Mol. Genet. 22, R77-87). In contrast, neurodegeneration has also been characterized prior to significant accumulation of detergent-insoluble TDP-43 species in other systems (Arnold, et al., (2013), Proc. Natl. Acad. Sci. USA 110, E736-45). TDP-43 sequestration within artificial inclusions recapitulates loss-of-function phenotypes (Prpar Mihevc, et al., (2016), Sci. Rep. 6, 33996) and TDP-43 knockout is embryonic lethal while conditional knockdown models produce ALS-like phenotypes in vivo (Xu, et al., (2014), Rare Dis. 2, e944443). This suggests that perturbations in TDP-43 function may also drive neurotoxicity. Data disclosed herein show that cytoplasmic 20 mislocalization precedes light-induced TDP-43 particle or inclusion formation and neuronal death, while neuroprotective oligonucleotide treatment delays/prevents these events. Thus, it is possible that cytoplasmic sequestration in the form of aberrant TDP-43 assemblies may produce neurodegeneration through both gain- and loss-of-function mechanisms.

In sum, disclosed herein is an optogenetic-based method to induce controlled TDP-43 proteinopathy in live cells, and it is established that RNA regulates the formation of TDP-43 inclusions. These studies show that aberrant phase transitions are toxic to human cortical-like neuronal cells. Finally, these data show bONs or bait nucleic acid (bNA) strategies can inhibit aberrant phase transitions of TDP-43, providing a therapeutic approach to disrupt pathological DNA/RNA binding protein inclusions.

### Supplemental Information

Chronic Blue Light Stimulation Alone does not Induce Mislocalization of Endogenous TDP-43.

HEK293 cells expressing Cry2olig-mCh were exposed to chronic blue light stimulation (16 h, ~0.3 mW/cm², 465 nm) and analyzed for mislocalization of endogenous TDP-43. Cells exposed to chronic blue light stimulation or darkness both showed no cytoplasmic mislocalization of endogenous TDP-43 by immunofluorescence. Cell nuclei were identified.

Cell lysates were collected following chronic blue light stimulation or darkness and separated into nuclear and cytoplasmic fractions. Western blot analysis of endogenous TDP-43 showed no cytoplasmic mislocalization of TDP-43 with or without blue light stimulation.

Chronic Blue Light Stimulation Promotes the Formation of High-Molecular-Weight optoTDP43 Oligomers and Recruitment of Non-Optogenetic TDP-43 to Inclusions.

Cell lysates from HEK293 cells expressing optoTDP43 were collected and analyzed by size exclusion chromatography. Samples were collected from cells either exposed to stimulation with blue light (16 h, ~0.3 mW/cm², 465 nm, bottom) or kept in darkness. The void volume of the column was determined with blue dextran (2000 kDa). Column fractions were subjected to SDS-PAGE and western blot analysis for TDP-43. The elution peak of column size standards (232 kDa and 60 kDa) were identified. optoTDP43 proteins exposed to blue light stimulation demonstrated a shift towards higher-molecular weight species, indicating light-induced oligomerization.

Lysates from optoTDP43-expressing cells exposed to chronic blue light treatment (16 h, ~0.3 mW/cm², 465 nm) or darkness were collected and separated into detergent-soluble and RIPA-insoluble, urea-soluble fractions. Western blot analysis of mCherry proteins showed an enhanced shift of full-length and n-terminal cleaved optoTDP43 products in the detergent-insoluble fraction.

ALS/FTLD patient tissue from the hippocampus and spinal cord was separated into detergent-soluble and RIPA-insoluble, urea-soluble fractions prior to western blot analysis of TDP-43.

Cell lysates collected from Cry2-mCh-expressing cells following chronic blue light stimulation or darkness were separated into detergent-soluble and RIPA-insoluble, urea-soluble fractions. Western blot analysis probing for endogenous TDP-43 showed no recruitment of TDP-43 to the insoluble fraction with or without chronic blue light stimulation.

To confirm the ability of optoTDP43 to recruit non-optogenetic TDP-43 species into light-induced inclusions, HEK293 cells were co-transfected with either optoTDP43 or the photoreceptor-only control Cry2olig-mCh and EGFP-TDP43. optoTDP43-expressing cells exposed to chronic blue light stimulation (16 h, approximately 0.3 mW/cm², 465 nm) showed co-localization of light-induced inclusions with EGFP-TDP43. Light-induced Cry2olig-mCh clusters showed no co-localization with EGFP-TDP43 signal, indicating a TDP-43:TDP-43 interaction-dependence of recruitment to induced optoTDP43 inclusions. Cell nuclei were identified.

Cells expressing optoTDP43 were exposed to chronic blue light stimulation (16 h, ~0.3 mW/cm², 465 nm) or darkness and immunostained for ubiquitin. Light-induced optoTDP43 inclusions strongly co-localized with ubiquitin signal. Cell nuclei were identified. optoLCD granules display properties of phase-separated droplets and full-length optoTDP43 does not form optoDroplets.

Images of individual optoLCD granules showing fusion events following acute blue light stimulation (1 sec, 1% laser power, 488 nm) were obtained. optoLCD granules underwent fusion and subsequent relaxation upon granule:granule contact, showing a liquid-like state of light-induced LCD granules.

Images of optoLCD particle formation following acute blue light stimulation (1 sec, 1% laser power, 488 nm) in cells expressing increasing concentrations of optoLCD proteins (as determined by relative fluorescence intensity) were obtained. As more protein was expressed, more optoLCD particle formation was observed.

Cells expressing optoLCD were exposed to acute blue light stimulation of increasing laser intensity (1 sec, 1-10% as indicated, 488 nm) and normalized granule number per cell was tracked over time. optoLCD proteins displayed enhanced phase transition responses following acute blue light stimulation of increasing light input, showing a tunable property of light-induced LCD phase transitions as a function of activated photoreceptor molecules.

Western blot analysis of WT and mutant optoLCD protein expression levels showed roughly comparable levels of expression for each protein.

Full-length optoTDP43 was expressed in HEK293 cells exposed to acute blue light stimulation (1 sec, 10% laser power, 488 nm) to determine whether full-length TDP-43 could undergo LIPS. No optoTDP43 granule formation was observed in response to acute blue light stimulation.

### Cry2Olig-LCD Confers an Enhanced Phase Separation Response to Light.

HEK293 cells expressing either optoLCD or the Cry2olig modified photoreceptive domain fused to the TDP-43 LCD (Cry2olig-LCD) were subjected to acute blue light stimulation (1 sec, 1% laser power, 488 nm) and granule formation was tracked over time.

Quantification of normalized granules per cell over time showed an enhanced phase transition response to light when the TDP-43 LCD is fused to the Cry2olig domain versus the WT Cry2 PHR domain.

Images of individual Cry2olig-LCD granules undergoing fusion events following acute blue light stimulation (1 sec, 1% laser power, 488 nm) were obtained. As was observed with optoLCD droplets, Cry2olig-LCD granules underwent fusion and relaxation upon granule:granule contact.

A Pearson's correlation was executed to analyze the protein concentration-dependence of Cry2olig-LCD phase transitions in response to acute blue light stimulation (1 sec, 10% laser power, 488 nm). Baseline fluorescence intensity was plotted against maximum granule number per cell during the imaging period. The Cry2olig-LCD concentration-dependence correlation had an r² of 0.874.

Cry2olig-LCD-expressing cells were exposed to acute blue light stimulation of increasing laser intensity (1 sec, 1-10% as indicated, 488 nm) and normalized granule number per cell was quantified post-stimulation. Again, an enhanced phase separation response was observed following blue light stimulation of increasing intensity.

Cells expressing a protein containing the TDP-43 RRMs with RNA-binding mutations 15 fused to the Cry2olig photoreceptor (RRMs 5FL) were exposed to acute blue light stimulation (1 sec, 10% laser power, 488 nm). No droplet formation was observed following light stimulation, showing the TDP-43 LCD facilitates LIPS behavior.

TDP-43 Inclusions in Patient Tissue and optoTDP43 System Show an Absence of RNA.

optoTDP43 was expressed in HEK293 cells that were exposed to chronic blue light stimulation (16 h, approximately 0.3 mW/cm², 465 nm) to induce optoTDP43 inclusion formation. Cells were fixed in ice-cold methanol and stained with SYTO RNAselect non-selective RNA dye to determine whether optoTDP43 inclusions contained any RNA species, in addition to mRNA. optoTDP43 inclusions did not co-stain with SYTO RNAselect dye, showing there were no RNA species contained within inclusions.

HEK293 cells expressing WT optoTDP43, RNA-binding-deficient optoTDP43 5FL, or non-optogenetic TDP43-mCh were chronically stimulated with blue light (approximately 0.3 mW/cm², 465 nm) and simultaneously imaged over time in an automated microscopy screen to assess whether RNA-binding affects full-length TDP-43 inclusion formation. Quantification of percentage of cells with inclusions over time showed a significantly increased rate of light-induced inclusion formation with reduced RNA-binding efficiency (optoTDP43 5FL).

Additional representative images of mRNA FISH analysis of ALS/FTLD patient tissue. Hippocampal and spinal cord sections from two FTLD cases (C90RF72-FTLD and FTLD-TDP43) and one ALS case (sporadic ALS) were examined by immunohistochemistry and RNA FISH. In all cases, no co-localization was observed between mRNA and pTDP-43 signal.

RNA-Deficient TDP-43 Inclusions do not Co-Localize with Stress Granule Components.

HEK293 cells expressing EGFP-TDP43cyto were immunostained for hyperphosphorylated TDP-43 or p62 and G3BP1 following sodium arsenite treatment, and representative images were obtained.

Images of cells containing TDP-43 5FL and TDP-43cyto 5FL inclusions immunostained for stress granule proteins G3BP1 and eIF4G were obtained.

HEK293 cells expressing optoTDP43 were subjected to sodium arsenite treatment, chronic blue light stimulation (16 hr, approximately 0.3 mW/cm², 465 nm), or darkness and immunostained for known stress granule components G3BP1 and eIF4G or ATXN2 and TIAR, and representative images were obtained.

Cells treated with sodium arsenite showed that optoTDP43 could be recruited to stress granules through the activation of endogenous cellular stress pathways. However, optoTDP43 inclusions induced with blue light stimulation did not co-localize with any of the tested stress granule markers.

Representative images were obtained of neuropathological examination of TDP-43 inclusions and SG component proteins (G3BP1; ATXN2) in FTLD-TDP and ALS/FTLD hippocampal sections. Co-localization between SG components and TDP-43 inclusions was observed.

### ReNcell VM Cortical Differentiation Yields a Highly Neuronally-Enriched Culture.

The ReNcell VM neuronal differentiation protocol schematic included a neurosphere preparation beginning on day−10. On day−4, neursphere dissociation occurred, and proliferation began on proliferation media (DMEM/F12+Glut, B27, Heparin (2 ng/mL), bFGF (20 μg/mL), and hEGF (20 μg/mL)). On day 0, neuronal differentiation occurred on Stage 1 Media (DMEM/F12+Glut, B27, GDNF (2 ng/mL), d,b,cAMP (1 mM)). On day 6, neuronal maintenance began on Stage II Media (DMEM/F12+Glut, B27, GDNF (2 ng/mL)).

Immunofluorescence images were obtained of ReNcell VM neurons (differentiation day 18) following the differentiation protocol described in the immediately preceding paragraph. MAP2 and βIII-tubulin were identified at 20× and 60× magnification.

The percentage of MAP2/βIII-tubulin double-labeled cells was quantified and showed a highly enriched (about 75%) neuronal population following the differentiation protocol.

ReNcell neurons were exposed to chronic blue light stimulation (48 hr, approximately 0.3 mW/cm², 465 nm) or darkness prior to examination of neuronal morphology and cell viability, as assessed by measurements of ATP levels (CellTiter-Glo, Promega). No differences in morphology or cell viability were observed between neurons in blue light or darkness conditions. Treatment with 0.1% Triton X-100 served as a positive death control.

### STAR Methods

### Experimental Model and Subject Details

### Cell Culture

HEK293 cells were maintained in DMEM (Thermo Fisher Scientific) supplemented with 10% HyClone Bovine Growth Serum (GE Healthcare Life Sciences) and 1× GlutaMAX (Thermo Fisher Scientific) at 37° C. and 5% CO₂. Cells were seeded onto coverslips or plates coated with collagen (50 μg/mL, Gibco) and allowed to incubate overnight prior to transfections (Lipofectamine 3000, Thermo Fisher Scientific) with 100 ng of DNA performed according to manufacturer's instructions. Cells were plated on collagen-coated glass bottom plates for live-cell imaging, high throughput screening, and FRAP analysis. All manipulations of cells expressing optogenetic constructs were performed under red lamp illumination to avoid ambient light exposure.

### Neuronal Progenitor Maintenance and Differentiation

ReNcell VM (Millipore) were maintained and differentiated according to previously described protocols but with minor modifications (Donato, et al., (2007), BMC Neurosci. 8, 36). In brief, cells were maintained in proliferation media (DMEM/F-12, 1× GlutaMAX™ supplemented with 1×B27 (Gibco), 2 ng/mL heparin (Sigma), 20 μg/mL bFGF (Millipore) and 20 μg/mL hEGF (Millipore). Differentiation was initiated by plating ReNcell VM onto low attachment plates in proliferation media in order to establish neurosphere production. Neurospheres were then mechanically dissociated and grown to full confluency. Cells were further differentiated in DMEM/F-12, supplemented with 1× GlutaMAX™, 1×B27, 2 ng/mL GDNF (PeproTech) and 1 mM diburtyrl-cAMP (Tocris) for one week and then maintained in DMEM/F-12 supplemented with 1× GlutaMAX™, 1×B27, and 2 ng/mL GDNF.

### Lentiviral Production and Transduction

Lentiviral transfer vectors encoding optoTDP43, Cry2-mCh or iRFP670 were co-transfected with packaging plasmids (OriGene) into HEK293T cells using the Turbofectin transfection reagent (OriGene) according to manufacturer's instructions. Following an initial media change, lentiviral supernatant was collected at 24 and 48 hr post-transfection prior to filtration and overnight incubation at 4° C. with 1× Lentivirus concentration solution (OriGene). The following day, concentrated lentiviral supernatant was centrifuged at 3,500×g for 25 min at 4° C. The resulting pellet was recentrifuged at 3,500×g for 5 min prior to re-suspension in ice-cold, sterile PBS. Pellets were then allowed to dissolve for 1-2 days at 4° C. Resuspended lentiviral particles were then aliquoted and titers were determined utilizing the One-Wash Lentivirus Titer Kit, HIV-1 p24 ELISA (OriGene). Neuron transductions were performed by diluting lentiviral particles at an MOI of 5 in neuronal differentiation media. Media changes were performed after 48 hr of incubation and all experiments were initiated at 96 hr post-transduction.

### Stress Treatments

Sodium arsenite (0.5 mM, 30 min) and heat shock (43° C., 45 min) treatment were used to induce stress granule formation where indicated.

### Blue Light Treatments

Blue light stimulation was performed in 24-well or 6-well plates using custom-built LED arrays designed to fit plate dimensions and withstand common temperature/humidity requirements of cell culture incubators. Individual LED diodes were positioned ~3.0 cm above the culture surface to provide a range of about 0.1-0.3 mW/cm² of 465 nm light stimulation to the cultured cells.

### RNA and Oligonucleotide Treatments

Total HEK293 RNA was extracted utilizing the miRNeasy RNA isolation kit (Qiagen) according to manufacturer's instructions. RNA oligonucleotides with 2′OMe modifications were synthesized by GenScript. For experiments utilizing HEK293 cells, total RNA (2.5 μg) or RNA oligonucleotides (200 nM-1Ip M as indicated) were transfected into cells using the Lipofectamine RNAiMAX reagent (Thermo Fisher) according to manufacturer's instructions. For ReNcell neuron experiments, oligonucleotides were diluted into normal differentiation medium at the indicated concentrations prior to direct addition to cells.

### Bacterial Growth and Protein Expression

For purification of MBP-tagged TDP-43 WT and 5FL proteins, BL21 (DE3) E. coli cell cultures were grown at 37° C. to an OD600 of 0.6-0.9 as previously reported (Wang, et al., (2018), EMBO J. 37). Protein expression was induced by IPTG (1 mM) prior to overnight incubation at 16° C. Cells were then harvested by centrifugation.

### Method Details

### Cloning

All full-length and truncated optoTDP43 plasmids were constructed by inserting PCR-generated fragments at the SmaI restriction enzyme site by Gibson Assembly (HiFi DNA Assembly Master Mix, NEB) of Cry2olig-mCh and Cry2 PHR-mCh base vectors (Plasmids 60032 and 26866, Addgene). TDP43-mCh was generated by inserting full-length TDP-43 CDS (synthesized by Genecopoeia) between the NheI and SmaI restriction enzyme sites of the Cry2olig-mCh backbone. All wild-type optoTDP43 vectors were constructed using the same TDP-43 insert. optoTDP43 constructs containing the TDP43cyto, 5FL and/or M337V point mutations were generated from mutant TDP43 plasmids (Plasmids 84912, 84914, 98674, Addgene). Constructs containing the fusRRM (WT or 4FL) were generated by three-fragment Gibson Assembly, inserting the fusRRM fragment and TDP-43 LCD simultaneously into the Cry2olig-mCh backbone at the SmaI restriction site. The plasmid encoding TDP as a C-terminal MBP-tagged protein (TDP43-MBP-His6) was purchased from Addgene (Plasmid 104480) and the 5FL mutant was generated via QuikChange Multi Site-directed Mutatagenesis (Agilent). EGFP-TDP43 constructs were generated by inserting the wild-type or mutant TDP-43 fragments between the HindIII and KpnI restriction enzyme sites of EGFP-alpha-synuclein vector (Plasmid 40822, Addgene). G3BP1-mCh was generated through the insertion of the full-length G3BP1 CDS (synthesized by Genecopoeia) into the mCherry2-C1 backbone (Plasmid 54563, Addgene) at the SmaI restriction site.

### Detergent Solubility Assay

Solubility of TDP-43 was assessed as previously described with minor modifications (van Eersel et al., PLOS ONE, (2011), 6(7):e22850). Cells were washed once with ice-cold PBS, lysed with modified RIPA buffer (25 mM Tris-HCl pH 7.6 (Sigma-Aldrich), 150 mM NaCl (Millipore Sigma), 5 mM EDTA (Sigma-Aldrich), 1% Triton X-100 (Sigma-Aldrich), 1% sodium deoxycholate (Sigma-Aldrich), 0.1% SDS (Fisher Scientific), protease inhibitor cocktail (Sigma-Aldrich), 1 mM PMSF (Thermo Fisher Scientific), phosphatase inhibitor cocktails 2 and 3 (Sigma-Aldrich)) and incubated on ice for 10 min. Following brief sonication on ice, lysates or homogenates were centrifuged for 1 hr at 100,000×g at 4° C. Supernatants were collected as the detergent-soluble fraction. Protein concentrations were determined using the RC DC protein assay (Bio-Rad). Pellets were then resuspended in RIPA buffer and re-sonicated and re-centrifuged. The resulting supernatant was discarded and pellets were resuspended in urea buffer (30 mM Tris pH 8.5 (Sigma-Aldrich), 7 M urea (Sigma-Aldrich), 2 M thiourea (Sigma-Aldrich), 4% CHAPS (Thermo Fisher Scientific), protease inhibitor cocktail (Sigma-Aldrich), 1 mM PMSF (Thermo Fisher Scientific), phosphatase inhibitor cocktails 2 and 3 (Sigma-Aldrich)). Following brief sonication on ice, lysates were centrifuged for 1 hr at 100,000×g at 22° C. This final supernatant was then collected as the detergent-insoluble, urea-soluble fraction. Proteins from each fraction were then separated by SDS-PAGE and analyzed by western blot analysis.

### Nuclear/Cytoplasmic Fractionation

Nuclear/cytoplasmic subcellular fractionation was performed utilizing NE-PER Nuclear and Cytoplasmic Extraction Reagents (Thermo Fisher Scientific) according to manufacturer's instructions. Protein concentrations for individual fractions were determined using the Pierce BCA Protein Assay Kit (Thermo Fisher Scientific) and subsequently analyzed by western blot.

### Size-Exclusion Chromatography

A 90 mL Sephacryl S-300 column equilibrated in 25 mM Tris pH 7.8, 150 mM NaCl, 5 mM EDTA, 0.5% Triton X-100, 0.5% deoxycholate was calibrated with the size standards Blue Dextran (2000 kDa), Catalase (232 kDa), and Hemoglobin (60 kDa). Lysates from cells expressing optoTDP43 which had been subjected to blue light stimulation or darkness were loaded in a 0.5 mL volume and the column was run at a flow rate of 0.3 mL/min at 4° C. An initial volume of 30 mL was passed through the column, which corresponded to the column void volume, then 2.5 mL fractions were collected. A sample from each fraction was mixed with SDS sample buffer and subjected to SDS-PAGE, followed by western blotting as described below.

### SDS-PAGE/Western Blotting

Prior to SDS-PAGE, protein concentrations were determined using the Pierce BCA Protein Assay (Thermo Fisher Scientific) or RC DC Protein Assay (Bio-Rad). Samples were separated by SDS-PAGE (4-20% Mini-PROTEAN TGX Precast Gels, Bio-Rad) and transferred to PVDF membranes (Immobilon-FL, EMD Millipore) using the Trans-Blot Turbo Transfer System (Bio-Rad). Following water and TBS washes, membranes were blocked in Odyssey Blocking Buffer (Li-Cor) for 1 hr at room temperature. Membranes were then washed and incubated with primary antibodies in TBS-T (0.1% Tween) supplemented with 50% blocking buffer overnight at 4° C. Primary antibody dilutions used were as follows: rabbit anti-TDP43 (Proteintech, 1:1000), mouse anti-α-tubulin (Sigma-Aldrich, 1:10000), rabbit anti-Lamin B1 (Abcam, 1:5000), rabbit anti-GAPDH (Sigma-Aldrich, 1:10000), mouse anti-mCherry (Novus Biologicals, 1:1000). Following TBS-T washes, membranes were incubated with secondary antibodies (Li-Cor, IRDye 680/800, 1:10000) for 1 hr at room temperature. Membranes were then washed with TBS-T and bands were visualized using the Odyssey CLx imaging system.

### Immunofluorescence

For standard immunofluorescence analysis, cells were then fixed with 4% PFA for 15 min at room temperature following one PBS wash. Following three additional PBS washes, cells were blocked with 5% normal donkey serum (NDS) in 0.3% Triton X-100 for 1 hr at room temperature. Primary antibodies were prepared in 1×PBS supplemented with 0.3% Triton X-100/5% NDS and cells were incubated in antibody solution overnight at 4° C. Primary antibody dilutions used were as follows: mouse anti-SQSTM1/p62 (Abcam, 1:100), rabbit anti-SQSTM1/p62 (Abcam, 1:500), rat anti-phospho TDP-43 (S409/410), Clone 1D3 (EMD Millipore, 1:200), rabbit anti-G3BP1 (Proteintech, 1:500), mouse anti-eIF4G (Santa Cruz, 1:300), rabbit anti-ATXN2 (Proteintech, 1:400), rabbit anti-TIAR (Santa Cruz, 1:300), mouse anti-digoxin (Jackson ImmunoResearch, 1:200), mouse anti-ubiquitin (Santa Cruz, 1:200). Following three PBS washes, secondary antibodies were diluted in 0.3% Triton X-100/5% NDS and incubated with cells for 1-2 hrs at room temperature. Cells were then washed and coverslips were mounted onto slides (ProLong Diamond Antifade Mounting Media with DAPI, Invitrogen) to be visualized by confocal microscopy.

For total RNA staining, cells were fixed in 100% methanol for 15 min at −20° C. Following three PBS washes, cells were incubated in SYTO® RNASelect™ Green Fluorescent Cell Stain (Thermo Fisher Scientific) staining solution (500 nM in PBS) for 20 min at room temperature. Cells were then washed three additional times in PBS prior to blocking and counterstaining as described above.

### Immunohistochemistry

Formalin-fixed, paraffin-embedded human hippocampus and cervical spinal cord sections of ALS/FTLD subjects retrieved from the Neurodegenerative Brain Bank at the University of Pittsburgh, following protocols approved by the University of Pittsburgh Committee for Oversight of Research and Clinical Training Involving Decedents (CORID). Following antigen retrieval with Target Retrieval Solution, pH 9 (Dako Agilent), immunofluorescence staining was performed using the following primary antibodies: TIA1 (1:1000, Abcam), Ataxin 2 (1:50, Proteintech), G3BP1 (1:100, Proteintech) and pTDP-43 (1D3, 1:500, provided by Manuela Neumann, Helmholtz Zentrum, Munich, Germany). Immunofluorescence signal was visualized using Alexa Fluor 488 and Cy3 labeled secondary antibodies (both 1:200, Jackson ImmunoResearch) and DAPI nuclear counterstain (1:1000, Thermo Scientific).

### RNA Fluorescent In Situ Hybridization (RNA FISH)

RNA-FISH using PolyT (TTTTTTTTTTTTTTTTTTTTTTTTTVN/3Dig_N) (Exiqon) or scramble control (5DigN/GTGTAACACGTCTATACGCCCA) (Batch 233334, Exiqon) probes was conducted as previously described but with minor modifications (Zhang, et al., (2015b), Nature 525, 56-61). In short, cells were fixed in 3.2% PFA (Electron Microscopy Science), permeabilized for 10 min in 0.3% Triton X-100 (Sigma-Aldrich), equilibrated for 10 min in 1×SSC (Thermo Fisher Scientific) and then incubated in 40% formamide (Sigma-Aldrich) at 55° C. for 10 min. Hybridization buffer (100% formamide, 1 mg/mL BSA Fraction V (Fisher Scientific), 20 mM ribonucleoside vanadyl complex (Sigma-Aldrich), 0.1M NaPO₄, 20×SSC) and then probe mixture (10 g/L salmon sperm (Thermo Fisher Scientific), 20 mg/mL E. Coli tRNA (Thermo Fisher Scientific), 80% formamide, 25 μM RNA-Probe preheated to 85° C.) were added to the cells for 2 hrs at 55° C. Next, the cells were washed with 40% formamide, 1×SSC, TBS-50 pH 7.4, 5M NaCl (Thermo Fisher Scientific Scientific) 1M Tris pH 7 (Thermo Fisher Scientific), 1M Tris, pH 8 (Thermo Fisher Scientific Scientific)), and then 1×PBS. Following a cross-linking step with 3.2% PFA and 0.3% Triton X-100, the cells were immunostained and washed with IF Buffer (TBS-50, 0.5 g BSA Fraction V, Protease-free BSA (Fisher Scientific)) and then incubated in blocking buffer (TBS-50, BSA Fraction V, 5% Normal Donkey Serum). Finally, the cells were incubated with primary antibodies at 4° C. overnight, and the following day were washed with IF buffer and incubated with secondary antibodies for 1 hr at room temperature. Additional washes with IF Buffer, TBS-50, MgCl₂, and 1×PBS were performed prior to mounting coverslips with ProLong Diamond Antifade Mounting Media and visualized by confocal microscopy.

### Cell Viability Assays

Analysis of cell viability was performed utilizing the CellTiter Glo Luminescent Cell Viability Assay (Promega) according to manufacturer's instructions. Treatment of cells with 0.1% Triton X-100 for 1 hr served as negative viability controls. Raw luminescence values were normalized to control group means and compared across experimental conditions.

### Microscopy

### Live-Cell Imaging

All live-cell imaging experiments were performed on a Nikon Al laser-scanning confocal microscope system outfitted with a Tokai HIT stagetop incubator utilizing 40× and/or 60×oil immersion objectives (CFI Plan Apo Lambda 60× Oil, Nikon; CFI Plan Fluor 40× Oil, Nikon). Following transfections and/or treatments, medium was changed to phenol red-free growth medium (Gibco) and cells were allowed to equilibrate on the preheated (37° C. and 5% CO₂) stagetop incubator for 10 min prior to imaging. Acute blue light stimulation was achieved by utilizing the 488 nm laser line and the stimulation module within Nikon Elements imaging software. Activation duration varied from 1-8 sec and laser power ranged from 1-20% as indicated in different experiments. Stimulation regions of interest (ROIs) were drawn over fields of view prior to image acquisition. Following 2-5 baseline images, laser stimulation was performed and cells were imaged for up to 1 hr post-activation.

Fluorescence Recovery after Photo-Bleaching (FRAP) Imaging

Initially, granules or inclusions were identified using a 60× oil immersion objective by confocal microscopy and a 2×2 μm² bleaching ROIs were drawn over objects of interest. Reference ROIs of the same size were drawn in adjacent, non-bleached cells to control for photo-bleaching. Following 2-5 baseline images, objects were bleached for 500 ms using 50% laser power (488 nm or 594 nm laser lines) and cells were imaged for up to 5 min post-bleaching without delay.

### High-Throughput LED Screening

Following transfection or transduction, plates were placed into a pre-warmed Tokai HIT stage-top incubator and allowed to equilibrate for 30 min prior to imaging. Custom 96-well LED arrays were positioned above wells to provide blue light stimulation (-0.3 mW, 465 nm) and were interfaced with Nikon Elements imaging software to cooperate with imaging protocols. Communication between the LED array and microscope occurred through a 5 V analog output transmitted from a Texas Instruments BNC-2110 triggering device. Voltage signals were interpreted by a microcontroller by taking real-time voltage measurements corresponding to a specific light group combination. Through the use of a map function, the software was able to assign a value to each light group and turn the group on or off following the reading of the corresponding value by the microcontroller. Automated imaging protocols were designed and executed utilizing the Jobs module within Nikon Elements imaging software. The microscope was programmed to perform three sequential baseline plate scans prior to light exposure. Epifluorescent images were acquired with a Prime 95B CMOS camera (Photometrics) using the ET-dsRED filter set (Chroma). Nine fields of view were imaged over time per well in duplicate per experiment per condition. Baseline images were acquired every 10 min for 30 min in total. This was followed by simultaneous blue light stimulation and image acquisition for up to 24 hrs. Every 10 min during the stimulation period, LED diodes were programmed to sequentially switch off and allow for image acquisition. Following completion of imaging within wells, LED diodes were programmed to switch back on to resume light stimulation and a subsequent set of diodes was programmed to turn off to allow for imaging. This pattern was repeated throughout the remainder of the imaged wells to complete the plate scan.

### Recombinant Protein Purification

MBP-tagged TDP-43 WT and 5FL proteins were purified as previously reported (Wang, et al., (2018), EMBO J. 37). Cells were harvested by centrifugation, resuspended in TDP-43 binding buffer (20 mM Tris-Cl pH 8.0, 1 M NaCl, 10 mM imidazole, 10% (v/v) glycerol, 1 mM DTT), supplemented with complete EDTA-free protease inhibitor cocktail, and lysed via sonication. The protein was purified over Ni-NTA agarose beads (Qiagen) and eluted from the beads using 20 mM Tris-Cl pH 8.0, 1 M NaCl, 300 mM imidazole, 10% (v/v) glycerol, 1 mM DTT. The eluate was further purified over amylose resin (NEB) and eluted using 20 mM Tris-Cl pH 8.0, 1 M NaCl, 10 mM maltose, 10% (v/v) glycerol, 1 mM DTT. The protein was concentrated, flash frozen in liquid N₂, and stored as aliquots in −80° C. until further use.

### In Vitro Phase Separation and Aggregation Assays

To induce formation of droplets, the proteins were first buffer exchanged into 20 mM HEPES (pH 7.4), 150 mM NaCl, and 1 mM DTT using a Micro Bio-Spin P-6 Gel column (Bio-Rad). The protein was then centrifuged at 16,000 rpm for 10 min to remove any preformed aggregates and the protein concentration was measured via a Bradford assay. LLPS was initiated with the addition of 10% dextran (final buffer conditions of 5 μM TDP43, 15 mM Hepes (pH 7.4), 150 mM NaCl, and 1 mM DTT with the indicated amounts of total yeast RNA) and droplets were imaged using DIC microscopy.

To measure aggregation kinetics, TDP-43 was thawed and centrifuged at 16,000 rpm for 10 min. Protein concentration was measured via Bradford and TDP-43 WT or 5FL were diluted to a final concentration of 5 μM (in 20 mM Hepes (pH 7.0), 150 mM NaCl, 1 mM DTT, with indicated quantities of RNA). Aggregation was initiated by cleavage of the MBP tag using 5 μg/mL TEV protease and monitored via turbidity measurements using a TECAN M1000 plate reader.

### Electron Microscopy

Transmission electron microscopy (TEM) of purified TDP-43 WT and 5FL aggregates was performed as described previously (Guo, et al., (2018), Cell 173, 677-692.e20). Following the aggregation assays described above, 10 μL of each sample was adsorbed onto 300-mesh Formvar/carbon-coated copper grids (Electron Microscopy Sciences, Hatfield, PA) and stained with 2% (w/v) uranyl acetate. Excess uranyl acetate solution was removed prior to drying of the grids. Samples were then imaged using a JEOL-1010T12 transmission electron microscope.

### Quantification and Statistical Analysis

### Image Quantification and Analysis

All image visualization and quantification was performed with Nikon Elements Imaging Software and analyses were performed in a double-blind fashion. For fixed-cell image quantification of stress granule co-localization and granule size, 4-5 fields of view were analyzed per condition. EGFP-TDP43 granules were examined and percentages of granules co-localizing with stress granule markers (G3BP1, eIF4G) were calculated. Maximum intensity projections for each field of view were generated and granule area was determined using automated object detection.

For FRAP imaging experiments, mean fluorescence intensity was tracked within bleaching ROIs over time. Intensity values were corrected for photo-bleaching utilizing reference ROIs drawn within adjacent, non-bleached cells in the imaging field. Fluorescence intensity values were converted to percentages of baseline (pre-bleach) fluorescence intensity means, with minimum fluorescence intensity values collected throughout the imaging period set to 0%, and percentage fluorescence recovery to baseline values was plotted over time.

Time-lapse image sequences acquired during high-throughput LED screening were analyzed utilizing automated object recognition to identify intracellular optoTDP43 particles and inclusions. Objects were defined by fluorescence intensity and object area thresholding. The number of cells containing detected objects were tracked over time and divided by the total number of cells within the imaging field to generate percentage of cells with inclusions for each timepoint within the imaging sequence. Analysis of neuronal survival in longitudinal imaging datasets was performed through monitoring of the far-red iRFP670 fluorescent reporter. Cell death was defined by somatic rounding, neurite retraction/blebbing and loss of fluorescence signal. Times-of-death were recorded as the last timepoint at which neurons were observed to be alive and were used to generate Kaplan-Meier survival curves.

Quantification of light-induced granule formation and dissociation was performed using the spot detection function within Nikon Elements imaging software. Granules were identified according to size and contrast thresholding. Granule number per cell was monitored over time in pre-identified cells that were labeled by an ROI. Raw granule number per cell values were first normalized to baseline values per cell over time. In order to calculate normalized granule number per cell over time, a weighted baseline intensity and cell area was determined. Baseline cell fluorescence intensity and area were determined from the image acquired prior to light stimulation and from this means were calculated across all experimental groups to control for differences in protein concentration and cell size. Total group baseline mean fluorescence intensity and cell area were then used to weight granule number values per cell by dividing individual cell values by total group means. For granule dissociation rate quantification, raw granule number per cell values were first normalized to baseline values. Normalized granule number values were then converted to percentages of each individual cell's maximum granule number per stimulation cycle. Mean percentages of cell maximum granule number were then plotted over time for each stimulation cycle.

### Statistics

Statistical significance was calculated by Graphpad Prism software (Version 7.03) and resulting P values less than or equal to 0.05 were deemed to be significant. Unpaired Student's t-tests were used to determine statistical significance in data sets comparing two variables. Two-way ANOVAs with Sidak Post-hoc analysis was used for comparisons of FRAP and granule formation/intracellular inclusion screening curves. Pearson's correlations were performed to determine r² values between data sets. Exponential decay nonlinear regression analysis was performed to compare optoLCD dissociation curves across groups. For survival analysis, Kaplan-Meier estimates were used to generate survival curves and Gehan-Breslow-Wilcoxon tests were used to compare across groups. Cumulative risk-of-death curves were generated as described previously (Malik et al., Elife, (2018), 7:e35977) using custom scripts in RStudio.

### Example 2. Poly-UG Nucleic Acid Substrates Prevent Formation of TDP-43 Inclusions

Live imaging was performed on HEK cells transfected with optoTDP-43 and exposed to blue light stimulation. At four hours into light stimulation, a synthesized UG RNA sequence repeated 12 times was added (“UG₁₂”; SEQ ID NO:10). Using automated quantitative analysis to identify cells with optoTDP-43 inclusions at each timepoint, FIG. 7 shows 50 nM UG1₂ RNA prevented formation of optoTDP-43 inclusions at each timepoint and the percent of cells without optoTDP-43 inclusions at the final timepoint was reduced by about 50% compared to mock control RNA.

### Example 3. Modified, Designed DNA Substrates Prevent Formation of TDP-43 Inclusions

A modified 2′OMe DNA oligonucleotide (SEQ ID NO: 11) was designed to bind TDP-43 based on other targeting 3′UTR RNA sequences. 2′-O-methylation (2′OMe) modifications enhance stability of oligonucleotides. HEK cells expressing full-length optoTDP43 (Cry2olig-TDP43-mCherry) were plated in 96-well plates and remained in the dark (light −) or exposed to blue light (light+) 8 hrs after transfection for 32 hours. HEK cell viability/health was determined using the CellTiter-Glo® assay to quantify intracellular ATP (energy production) as a function of relative luminescence (RLU). Cells expressing optoTDP-43 but pretreated with the TDP-43 targeting/binding oligonucleotide Stem Loop TG24 (SL_TG24; SEQ ID NO: 11) exhibited a complete rescue of cell viability at 50 nM and 100 nM pretreatment in a dose-dependent manner (FIG. 8). Oligonucleotides consisting of 12 repeating adenine-cytosine (AC) motifs with a 2′OMe modified backbone fused to the same stem structure as SL_TG24 (AC24; SEQ ID NO:120) did not bind TDP-43 and failed to rescue TDP-43 inclusions-induced cytotoxicity at 50 nM and 100 nM pretreatment. For statistical analysis, a one-way ANOVA was performed with a Tukey's multiple comparisons posthoc. (*, p<0.05; **, p<0.01, ****, p<0.0001). These results show that modified DNA inhibitors can prevent formation of TDP-43 inclusions.

### Example 4. OptoTDP43 Model Comprising V5 Tag and VVD Light-Inducible Dimerization Protein

An additional optoTDP43 model construct was constructed in which full-length TDP-43 was used. A V5 tag (GKPIPNPLLGLDST; an amino acid sequence of viral RNA polymerase commonly used for tagging recombinant proteins) was N-terminally fused to full-length TDP-43. The C-terminal end of TDP-43 was fused to the about 64 kDa Vivid (VVD) protein, a Neurospora crassa fungal photoreceptor which dimerizes in response to blue light. VVD has been used as a light-inducible dimerization domain fused to target proteins to induce dimerization (Zoltowski, Biochemistry, (2008), 47(27):7012-7019). The V5-TDP43-VVD fusion construct was placed under doxycycline-inducible expression control using a Tet expression cassette. SEQ ID NO:6 is a polynucleotide sequence encoding V5-TDP43-VVD. SEQ ID NO:7 is a polypeptide sequence comprising V5-TDP43-VVD.

HEK293 cells were transfected with a V5-TDP43-VVD construct and incubated for 20 16 hrs. 100 ng/mL doxycycline were added to the media, and HEK293 cells expressing V5-TDP43-VVD were exposed to 84 h blue light stimulation. Cells were fixed and lysates were collected. Cells showed light-induced assemblies following fixation and immunofluorescence for the n-terminal V5 tag. Further, light-induced V5-TDP43-VVD assemblies also co-localized with phospho-TDP43 signal, resembling the pathological hallmarks of TDP-43 proteinopathy. Co-localization with phospho-TDP43 signal was not observed for cells expressing V5-TDP43-VVD but kept in the dark. Lysates of HEK293 cells exposed to 84 hrs of light or kept in the dark were then separated into detergent-soluble and insoluble fractions prior to SDS-PAGE and immunoblotting for total TDP-43. Cells exposed to blue light exhibited a shift of both exogenous V5-TDP43-VVD and endogenous TDP-43 into the insoluble fraction. These results show that the V5-TDP43-VVD construct inducibly forms aggregates and recapitulates TDP-43 proteinopathies in neurodegenerative disease.

An additional optoTDP43 model construct was constructed in which full-length TDP-43 was N-terminally fused with VVD protein and C-terminally fused with a V5 tag (henceforth, “VVD-TDP43-V5”), and placed under doxycycline-inducible expression control using a Tet expression cassette. SEQ ID NO:8 is a polynucleotide sequence encoding VVD-TDP43-V5. SEQ ID NO:9 is a polypeptide sequence comprising VVD-TDP43-V5.

HEK293 cells were transfected with a VVD-TDP43-V5 construct and incubated for 16 hrs. 100 ng/mL doxycycline were added to the media, and HEK293 cells expressing VVD-TDP43-V5 were exposed to 24 h of blue light stimulation. Cells were fixed and lysates were collected. After only 24 hrs of blue light exposure, cells showed light-induced assemblies following fixation and immunofluorescence for the n-terminal V5 tag. Quantification of normalized aggregation area demonstrated a significant induction of VVD-TDP43-V5 inclusion formation following blue light exposure compared to cells kept in darkness (FIG. 9). A dose-dependent effect was also observed between low doxycycline treatments (10 ng/mL) and high doxycycline treatment (1000 ng/mL), producing relatively low and high levels of protein expression respectively during light stimulation. Further, light-induced VVD-TDP43-V5 assemblies also co-localized with phospho-TDP43 signal, resembling the pathological hallmarks of TDP-43 proteinopathy. Co-localization with phospho-TDP43 signal was not observed for cells expressing VVD-TDP43-V5 but kept in the dark. These results show that the VVD-TDP43-V5 construct inducibly forms aggregates and recapitulates TDP-43 proteinopathies in neurodegenerative disease.

### Example 5. Prevention of and Disaggregation of FUS Protein Aggregates In Vitro

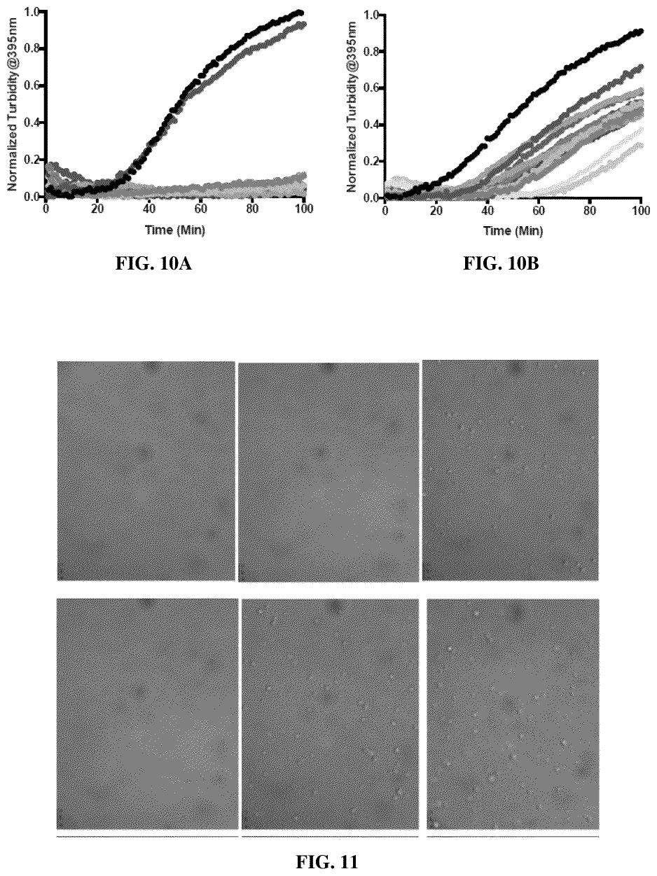

Full-length FUS protein was N-terminally fused with a TEV cysteine protease cleavage recognition sequence from Tobacco Etch Virus nuclear-inclusion-a endopeptidase. A Glutathione S-transferase (GST) tag was also N-terminally fused to the TEV cleavage recognition sequence, resulting in a GST-TEV-FUS construct. Upon expression and purification, GST-TEV-FUS (5 μM) was incubated with TEV protease (to cleave the GST-TEV motif) in the presence or absence of 20 μM specific RNAs known to bind FUS protein (for example, the polynucleotide sequence GGUG is a known FUS-binding RNA sequence motif). RNasin was also used to protect against RNA degradation. Samples were measured for turbidity every minute to assess the extent of aggregation. Turbidity results showed a subset of RNAs which were strong inhibitors of aggregation (FIG. 10A) and a subset of effective but intermediate inhibitors of aggregation (FIG. 10B). Strong RNA inhibitors essentially completely inhibited increases in turbidity while intermediate inhibitors partially inhibited turbidity increases. Strong inhibitors included RNA7 (SEQ ID NO:97), RNA8 (SEQ ID NO:98), RNA15 (SEQ ID NO:96), RNA19 (SEQ ID NO:99), RNA25 (SEQ ID NO:100), RNA26 (SEQ ID NO:101), RNA31 (SEQ ID NO:102), and RNA32 (SEQ ID NO:103). Intermediate inhibitors included RNA1 (SEQ ID NO:104), RNA2 (SEQ ID NO:105), RNA9 (SEQ ID NO:106), RNA10 (SEQ ID NO:107), RNA11 (SEQ ID NO:108), RNA13 (SEQ ID NO:109), RNA14 (SEQ ID NO:110), RNA17 (SEQ ID NO:111), RNA27 (SEQ ID NO:112), RNA28 (SEQ ID NO:113), RNA29 (SEQ ID NO:114), and RNA30 (SEQ ID NO:115).

To determine whether the selected RNA inhibitors could inhibit FUS phase transition, aggregation, or both, GST-TEV-FUS (5 μM) was incubated with TEV protease in the presence or absence of selected RNA inhibitors (20 μM) for 0-90 min. At 0, 30, 60, and 90 minutes, reactions were processed for sedimentation analysis. Pellet and supernatant fractions were resolved by SDS-PAGE and stained with Coomassie Brilliant Blue. The amount of FUS in the pellet fraction was determined by densitometry in comparison to known quantities of FUS. Results showed that RNA9 had little effect on reducing the amount of FUS found in the pellet fraction (representing protein aggregates) by 90 minutes, whereas RNA15 and RNA8 reduced the percentage of FUS in the pellet fraction by about 40% and 60%, respectively, by 90 minutes. RNA19 had intermediate effects (about 50% reduction by 30 minutes but about 10% reduction by 90 minutes) on reducing the amount of FUS found in the pellet fraction.

Fibrillization reactions were performed as described in the immediately preceding paragraph and processed for electron microscopy (EM) at the end of the reaction. Images identified regions of high-density stain that indicated formation of protein phases having high protein concentration. Differential interference contrast (DIC) images of GST-TEV-FUS (5 μM) droplets formed in the presence and absence of the specific RNAs (20 μM) were also obtained. Results showed that RNA8, RNA15, and RNA19 significantly reduced droplet formation, whereas droplet formation in the RNA9 sample was not substantially changed from that of an untreated sample (FIG. 11). These results show that strong RNA inhibitors prevent phase transition and aggregation, while intermediate RNA inhibitors prevent aggregation only.

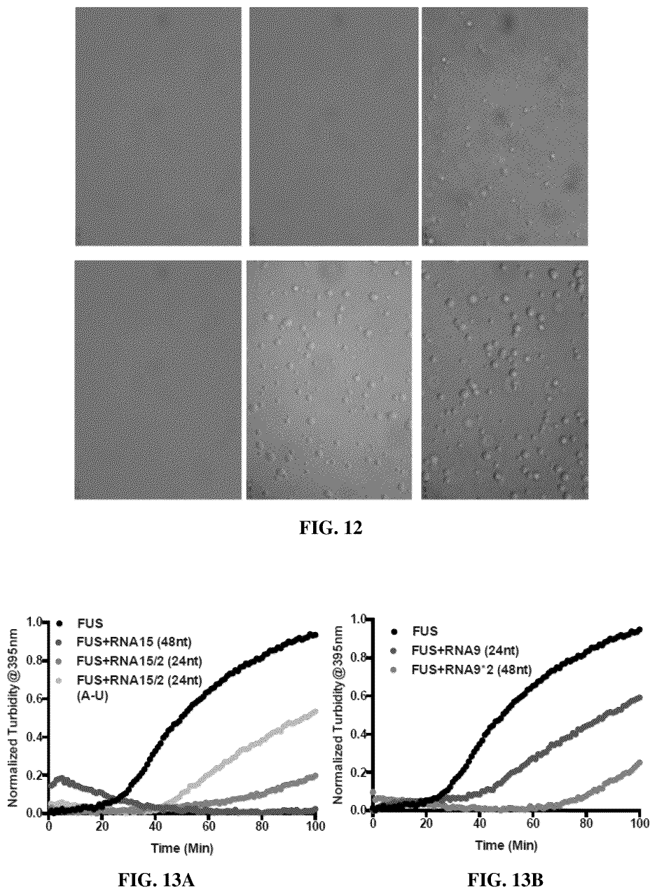

RNA inhibitors were next evaluated for their ability to disaggregate FUS aggregation and phase transition. GST-TEV-FUS (5 μM) droplets were first formed, and then selected RNA inhibitors were added to the protein droplets. DIC images showed that RNA8, RNA15, and RNA19 (strong inhibitors) essentially eliminated (disaggregated) all FUS protein droplets, whereas RNA9 (an intermediate inhibitor) modestly reduced the size and overall number of FUS protein droplets (FIG. 12). Fibrillization reactions were performed and, at the end of the reaction, water or RNA inhibitors (20 μM) were added to the reactions. Turbidity measurements were taken every minute to assess the extent of disaggregation. Results showed that strong RNA inhibitors RNA7 (SEQ ID NO:97), RNA8 (SEQ ID NO:98), RNA15 (SEQ ID NO:96), RNA19 (SEQ ID NO:99), RNA25 (SEQ ID NO:100), RNA26 (SEQ ID NO:101), RNA31 (SEQ ID NO:102), and RNA32 (SEQ ID NO:103) significantly reduced turbidity of FUS samples. Intermediate RNA inhibitors RNA1 (SEQ ID NO:104), RNA2 (SEQ ID NO:105), RNA9 (SEQ ID NO:106), RNA10 (SEQ ID NO:107), RNA11 (SEQ ID NO:108), RNA13 (SEQ ID NO:109), RNA14 (SEQ ID NO:110), RNA17 (SEQ ID NO:111), RNA27 (SEQ ID NO:112), RNA28 (SEQ ID NO:113), RNA29 (SEQ ID NO:114), and RNA30 (SEQ ID NO:115) also reduced turbidity of FUS samples, although to a lesser degree than strong RNA inhibitors. Disaggregation reactions were also processed for EM imaging at 20 and 120 minutes post-addition of RNA inhibitors. Results showed that RNA8, RNA15, and RNA19 significantly reduced the amount of dark staining in the images, demonstrating reduced FUS protein aggregation.

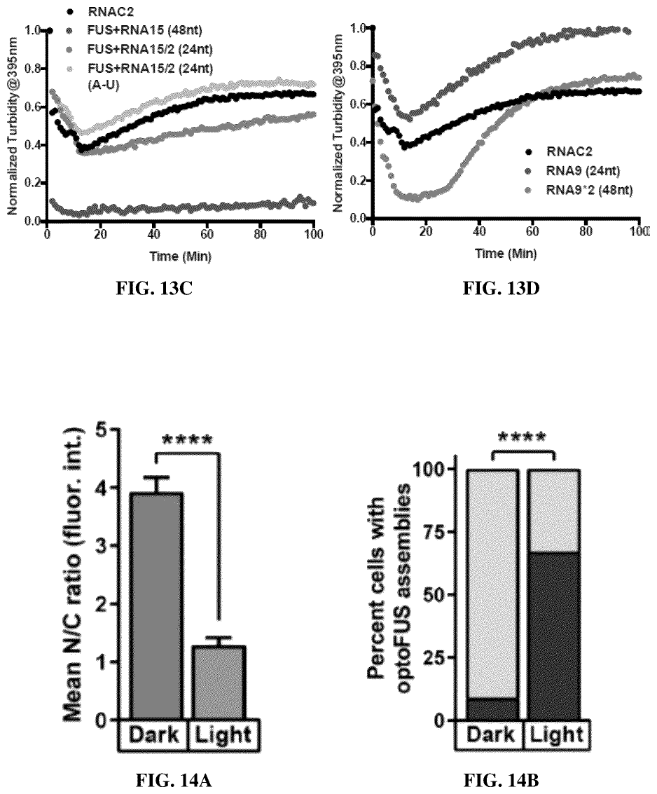

Next, inhibition activity of RNA inhibitors was assessed depending on the length and sequence of the RNA. Fibrillization reactions were performed by incubating GST-TEV-FUS (5 μM) with TEV protease in the presence or absence of selected RNA inhibitors (20 μM) for 0-100 min. Turbidity measurements were taken every minute to assess the extent of aggregation (and prevention thereof). When the strong inhibitor RNA15 (48 nucleotides) was shortened to 24 nucleotides, the shortened RNA became the intermediate inhibitor RNA22 (SEQ ID NO:117; “FUS+RNA15/2”) (FIG. 13A). When the length of the intermediate inhibitor RNA9 (24 nucleotides) was doubled to 48 nucleotides, the resultant RNA (“RNA9\hat{ }2 ”) (SEQ ID NO: 119) had increased inhibition activity (FIG. 13B). A single A to U mutation was introduced in RNA22, which also introduced a known FUS binding motif GUGGU into the sequence, resulting in RNA15/2 (A-U) (SEQ ID NO: 118). Interestingly, this single mutation in RNA15/2 (A-U) significantly weakened its inhibition activity compared to RNA22, indicating the specific RNA sequence is important for inhibition activity (FIG. 13A).

To test disaggregation potential of the RNA inhibitors, fibrillization reactions were repeated and, at the end of the reaction, selected RNA inhibitors (20 μM) were added to the reaction. Turbidity measurements were taken every minute to assess the extent of disaggregation. As with prevention of aggregation, disaggregation activity also depended on the length and sequence of the RNA. For example, RNA15 completely disaggregated pre-formed FUS aggregates, while the shortened version of RNA15 (RNA22) did not show disaggregation activity compared to the control RNAC2 (FIG. 13C). On the other hand, doubling the length of RNA9 (which did not have disaggregating activity) to result in RNA9\hat{ }2 rendered RNA9\hat{ }2 able to disaggregate pre-formed FUS aggregates (FIG. 13D). These results showed that RNA inhibitors can disaggregate existing FUS aggregates, but that the length and specific sequence of the RNA determines the extent of disaggregation activity.

Next, RNA inhibitors were assessed for inhibition activity when the RNA Recognition Motif (RRM) of FUS was mutated. Mutations in the FUS RRM reduce the binding affinity between RNA and FUS. Specifically, four phenylalanine (F) to leucine (L) mutations at positions 305, 341, 359, and 368 of FUS (FUS^{F305L:F341L:F359L:F368L}; henceforth known as “FUS 4FL”) are known to disrupt RNA binding (Daigle et al., Hum. Mol. Genet. (2013), 22(6):1193-1205). 8 nM fluorescein-labeled RNA inhibitors were combined with varying concentrations of GST-TEV-FUS or GST-TEV-FUS 4FL, and anisotropy of RNA binding was measured. Results showed that mutations in the FUS RRM (FUS 4FL) reduced binding affinity between RNA inhibitors and FUS. For example, the dissociation constant (Kd) for binding to WT FUS vs. FUS 4FL for RNA8 was 40.82±8.32 nM and 113.1±54.36 nM, respectively; for RNA15 was 104.7±9.2 nM and 769.0±281.2 nM, respectively; and for RNA19 was 101.5±28.1 nM and 296.6±132.6 nM, respectively.

To assess the requirement of RNA-binding for prevention of aggregate formation, RNA-binding deficient mutant GST-TEV-FUS 4FL (5 μM) was incubated with TEV protease in the presence or absence of separate strong inhibitors RNA8, RNA15, RNA19, or RNA31 (20 μM each) for 0-180 min. Turbidity measurements were taken every minute to assess the extent of aggregation. Each of the strong inhibitors failed to significantly reduce turbidity of FUS 4FL protein, demonstrating that RNA binding is required to prevent FUS aggregation.

To assess the requirement of RNA-binding for disaggregation, fibrillization reactions were performed and, at the end of the reaction, water or separate RNA inhibitors RNA8, RNA15, RNA19, RNA31, or RNA32 (20 μM each) were added to the reaction. Turbidity measurements were taken every minute to assess the extent of disaggregation. Results showed that RNA8, RNA15, RNA19, RNA31 each failed to disaggregate FUS 4FL (as measured by turbidity reduction) by 100 minutes. RNA32 modestly reduced FUS 4FL aggregation below control RNA RNAC2 and water alone, perhaps indicating some residual binding of this RNA to FUS 4FL. These results demonstrate that RNA binding is required to disaggregate existing FUS aggregates.

### Example 6. Construction and Characterization of an optoFUS Inducible Model of FUS Proteinopathy

Photoresponsive mCherry (mCh) protein was fused to the N-terminus of full-length FUS protein, and Cry2 was fused to the N-terminus of mCherry. The resultant construct Cry2-mCh-FUS was placed under the inducible control of a doxycycline-responsive Tet expression cassette. HEK293 cells were transfected with the construct and, after 16 hrs of incubation, doxycycline (5 ng/mL) were added to the media. Cells were exposed to blue light or darkness for 8 hrs, then fixed for fluorescence imaging. Significantly more optoFUS assemblies were readily visible in cells exposed to blue light than in cells exposed to darkness. The ratio of nuclear to cytoplasmic optoFUS was quantified and showed that significantly more optoFUS was found in the cytoplasm than the nucleus of cells exposed to light, as compared to cells maintained in darkness (FIG. 14A). Further, cells exposed to light showed a significantly increased proportion of transfected cells with optoFUS assemblies (FIG. 14B).

To test whether light-induced optoFUS assemblies were insoluble inclusions in cells, HEK293 cells expressing optoFUS were exposed to 8 h of darkness or blue light prior to fluorescence recovery after photobleaching (FRAP) analysis of optoFUS assemblies. Light-induced optoFUS assemblies show extremely little fluorescence recovery following bleaching, showing formation of static, immobile inclusions (FIG. 14C). To the contrary, cells exposed to darkness exhibited extensive fluorescence recovery following bleaching, showing dynamic assemblies. HEK293 cells expressing optoFUS were then exposed to 16 h of darkness or blue light prior to fractionation into soluble supernatant and insoluble pellet fractions. Extracts were then separated on a Western blot and probed using anti-mCh or an anti-FUS antibodies. Cells treated with blue light showed dose-dependent increases in optoFUS protein present within sarkosyl-insoluble, urea-soluble fractions at low (10 ng/mL) and high (100 ng/mL) doxycycline concentrations. Increases in endogenous FUS protein were similarly observed within sarkosyl-insoluble fractions in cells treated with high doxycycline and blue light. However, the majority of optoFUS protein and endogenous FUS protein in cells exposed to darkness remained in the soluble supernatant. These results show that light-induced optoFUS assemblies are insoluble inclusions similar to those found in FUS proteinopathy.

To test whether optoFUS inclusions recapitulate ALS-like pathologies, HEK293 cells expressing optoFUS were exposed to 8 h of darkness or blue light prior to immunofluorescence analysis of pathological hallmarks of FUS proteinopathy observed in ALS and FTLD. optoFUS inclusions were negative for FET family protein markers TAF15 and EWSR1, which are commonly found in FTLD, but not ALS, patient tissue inclusions. Further, fluorescence image analysis also showed co-localization of optoFUS inclusions with the 9G6 methylated FUS antibody, indicating an ALS-like methylation-status of optoFUS inclusions. These results show optoFUS inclusions recapitulate ALS-like pathological hallmarks.

### Example 7. RNA Inhibits optoFUS Proteinopathy in Cells

optoFUS was shown to form aggregates resembling those of FUS-related proteinopathy, and RNA inhibitors were shown to both prevent and disaggregate such optoFUS aggregates. As such, the model was examined in live cells to determine whether bait RNA oligonucleotides (bONs) could prevent and disaggregate optoFUS inclusions.

A 25-nucleotide bait RNA referred to as RNAS1 (SEQ ID NO:98) was constructed for cellular experiments. RNAS1 contains a known FUS binding motif GGUG polynucleotide sequence. A 24-nucleotide control RNA (SEQ ID NO:121) was also constructed and used for cellular experiments. HEK293 cells were transfected with the optoFUS construct, incubated for 16 hrs, and then doxycycline (5 ng/mL) and 500 nM, 1 μM, or 2.5 μM of either bait RNAS1 or control RNA were added to the media. Cells were then incubated under either blue light or darkness for 8 hrs and then fixed for analysis. Cells were fluorescently imaged and quantified for amount of aggregation observed. While no significant changes in optoFUS inclusion formation were observed following treatment with control RNA oligonucleotides, treatment with bait S1 oligonucleotides resulted in a dose-dependent inhibition of optoFUS inclusion formation (FIG. 15). These results show that bONs can prevent optoFUS inclusions in cells.

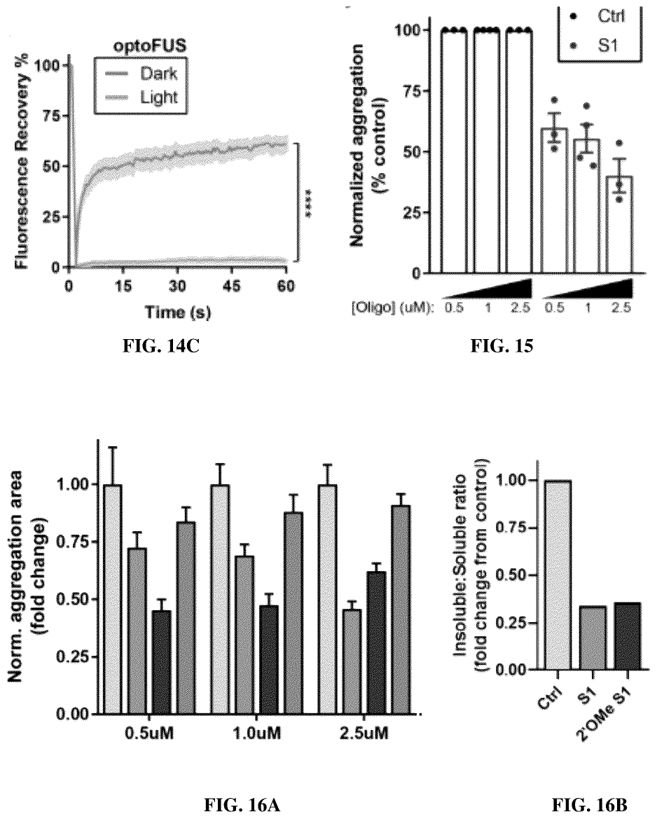

It was then tested whether modified RNAs could also prevent optoFUS inclusion formation in cells. Thus, experiments were repeated using the additional bONs RNA2′S1 (SEQ ID NO:116), which contained 2′OMe (a methyl group covalently linked to the 2′ hydroxyl of the ribose moiety) modifications at each nucleotide, and RNAPS-2′S1 (SEQ ID NO:116; identical to RNA2′S1 but containing both phosphorothioate bonds (PS) and 2′OMe modifications at each nucleotide). Both RNA2′S1, RNAPS-2′S1 contained the same nucleotide sequence as RNAS1 (with the exception of the noted modifications). HEK293 25 cells were transfected with the optoFUS construct, incubated for 16 hrs, and then doxycycline (5 ng/mL) and 500 nM, 1 μM, or 2.5 μM of either bait RNAS1, RNA2′S1, RNAPS-2′S1, or control RNA were added to the media. Cells were then incubated under either blue light or darkness for 8 hrs and then fixed for immunofluorescence analysis. Cells were fluorescently imaged and quantified for amount of aggregation observed. Results showed that HEK293 cells expressing optoFUS pre-treated with RNAS1 and RNA2′S1 exhibited significant inhibition of optoFUS inclusion formation, whereas control RNA and RNAPS-2′S1 oligonucleotides showed little inhibitory effect (FIG. 16A). Cells were then lysed and lysates were collected and separated into RIPA-soluble, and RIPA-insoluble, urea soluble fractions prior to SDS-PAGE and immunoblotting for optoFUS. Densitometry analysis of Western blots showed that a much greater percentage of RNAS1- and RNA2′S1-treated samples had soluble optoFUS protein compared to control RNA-treated samples (FIG. 16B). These results show that bONs can prevent optoFUS inclusion formation in cells.

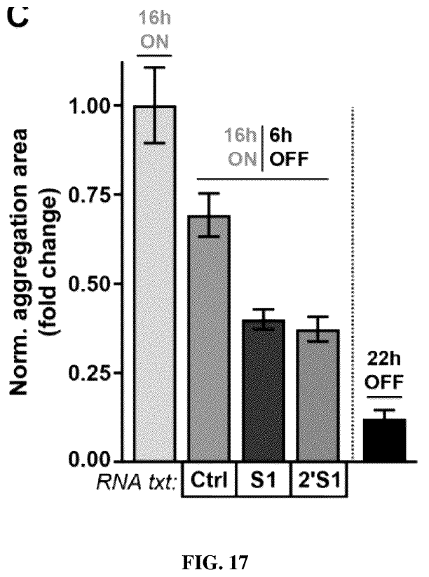

It was then tested whether modified RNAs could also disaggregate existing optoFUS inclusions in cells. The bait oligonucleotides (bONs) RNAS1 (SEQ ID NO:98) and RNA2′S1 (SEQ ID NO:116), as well as control RNA, were used in experiments. HEK293 cells were transfected with the optoFUS construct, incubated for 24 hrs, and then doxycycline (5 ng/mL) and AraC (to slow cell proliferation, thereby inhibiting exogenously expressed optoFUS) were added to the media. Cells were then incubated under either blue light or darkness for 16 hrs. 1 μM of either RNAS1, RNA2′S1, or control RNA were then added to cells, and a sample of cells were fixed for immunofluorescence analysis. The remainder of the cells treated with light were then incubated in darkness for 6 hrs (16 hrs light/6 hrs darkness), and then fixed for immunofluorescence analysis. Cells were fluorescently imaged and quantified for amount of aggregation observed. Quantification of aggregation normalized to untreated (no RNA) controls showed treatment with both RNAS1 and RNA2′S1 significant increased optoFUS inclusion clearance compared to control RNA treatment, demonstrating enhanced disassembly of pre-formed optoFUS inclusions (FIG. 17). These results show that bONs can disaggregate optoFUS inclusions in cells.

In the disclosed sequences, the term “m” appearing after any nucleotide in a sequences refers to 2′OMe modification of that nucleotide. While all disclosed polynucleotide sequences of polynucleotides that binds a nucleic acid-binding polypeptide can be used singly or repeated one or more times in a sequence, the polynucleotide sequences of SEQ ID NO:33-56 are well-suited for use singly or as repeated motifs in a polynucleotide that binds a nucleic acid-binding polypeptide.

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

1. A method to treat a neurodegenerative disease comprising administering to a subject with a neurodegenerative disease a therapeutically effective amount of a polynucleotide that binds a nucleic acid-binding polypeptide, wherein the nucleic acid-binding polypeptide can aggregate in cells and is associated with a neurodegenerative disease.

2. The method of claim 1, wherein the neurodegenerative disease is selected from Limbic-predominant Age-related TDP-43 Encephalopathy (LATE), Multisystem Proteinopathy, Amyotrophic Lateral Sclerosis, Frontotemporal Dementia, Alzheimer's Disease, Chronic Traumatic Encephalopathy, Traumatic Brain Injury, Cortical Basal Degeneration, and Huntington's Disease.

3. (canceled)

4. The method of claim 1, wherein the polynucleotide is administered in a pharmaceutical composition comprising a pharmaceutically acceptable excipient.

5. The method of claim 1, wherein the method reduces a rate of aggregation or an amount of aggregate of the nucleic acid-binding polypeptide in the subject.

6. (canceled)

7. The method of claim 1, wherein the nucleic acid-binding polypeptide is selected from TDP-43, FUS, HNR NPA1, HNR NPA2/B1, EWSR1, TIA-1, TTP, TAF15, MATR3, ATXN2, OPTN, Tau, HTT, and biologically active fragments and variants thereof.

8. (canceled)

9. The method of claim 1, wherein the polynucleotide comprises a synthetic RNA or a modified RNA.

10. The method of claim 1, wherein the polynucleotide comprises at least 24 consecutive nucleotides comprising at least 60% uracil and guanine nucleotides.

11. A method to inhibit protein aggregation in a neuronal cell comprising contacting the neuronal cell with a composition comprising a polynucleotide that binds a nucleic acid-binding polypeptide, wherein the nucleic acid-binding polypeptide can aggregate in cells and is associated with a neurodegenerative disease.

12. The method of claim 11, wherein the method reduces a rate of aggregation or an amount of aggregate of the nucleic acid-binding polypeptide in the cell compared to a control.

13. (canceled)

14. The method of claim 11, wherein the polynucleotide comprises any one of SEQ ID NO:10-84.

15. The method of claim 11, wherein the polynucleotide comprises
SEQ ID NO:85, SEQ ID NO:62, and SEQ ID NO:86, in that order;
SEQ ID NO:87, SEQ ID NO:63, and SEQ ID NO:88, in that order;
SEQ ID NO:92, SEQ ID NO:64, and SEQ ID NO:92, in that order;
SEQ ID NO:92, SEQ ID NO:65, and SEQ ID NO:92, in that order;
SEQ ID NO: 92, SEQ ID NO:66, and SEQ ID NO:92, in that order;
SEQ ID NO:92, SEQ ID NO:67, and SEQ ID NO:92, in that order;
SEQ ID NO:92, SEQ ID NO:68, and SEQ ID NO:92, in that order;
SEQ ID NO:92, SEQ ID NO:69, and SEQ ID NO:92, in that order;
SEQ ID NO:92, SEQ ID NO:70, and SEQ ID NO:92, in that order;
SEQ ID NO:92, SEQ ID NO:71, and SEQ ID NO:92, in that order;
SEQ ID NO:92, SEQ ID NO:72, and SEQ ID NO:92, in that order;
SEQ ID NO:92, SEQ ID NO:73, and SEQ ID NO:92, in that order;
SEQ ID NO:92, UGUGUGUG, and SEQ ID NO:92, in that order;
SEQ ID NO:92, UGUGUG, and SEQ ID NO:92, in that order;
SEQ ID NO:89, SEQ ID NO:74, and SEQ ID NO:89, in that order;
SEQ ID NO:90, SEQ ID NO:74, and SEQ ID NO:90, in that order;
SEQ ID NO:91, SEQ ID NO:74, and SEQ ID NO:91, in that order;
SEQ ID NO:92, SEQ ID NO:74, and SEQ ID NO:92, in that order;
SEQ ID NO:93, SEQ ID NO:74, and SEQ ID NO:93, in that order;
SEQ ID NO:94, SEQ ID NO:74, and SEQ ID NO:94, in that order;
SEQ ID NO:95, SEQ ID NO:74, and SEQ ID NO:95, in that order;
CGCGCGCG, SEQ ID NO:74, and CGCGCGCG, in that order;
CGCGCG, SEQ ID NO:74, and CGCGCG, in that order;
CGCG, SEQ ID NO:74, and CGCG, in that order:
CG, SEQ ID NO: 74, and CG, in that order;
SEQ ID NO:92, SEQ ID NO:75, and SEQ ID NO:92, in that order;
SEQ ID NO:92, SEQ ID NO:76, and SEQ ID NO:92, in that order;
SEQ ID NO:92, SEQ ID NO:77, and SEQ ID NO:92, in that order;
SEQ ID NO:92, SEQ ID NO:78, and SEQ ID NO:92, in that order;
SEQ ID NO:92, SEQ ID NO:79, and SEQ ID NO:92, in that order;
SEQ ID NO:92, SEQ ID NO:80, and SEQ ID NO:92, in that order;
SEQ ID NO:92, SEQ ID NO:81, and SEQ ID NO:92, in that order;
SEQ ID NO:92, SEQ ID NO:82, and SEQ ID NO:92, in that order;
SEQ ID NO:92, SEQ ID NO:83, and SEQ ID NO:92, in that order;
SEQ ID NO:92, SEQ ID NO:84, and SEQ ID NO:92, in that order;
SEQ ID NO:92, TGTGTGTG, and SEQ ID NO:92, in that order; or
SEQ ID NO:92, TGTGTG, and SEQ ID NO:92, in that order.

16. The method of claim 11, wherein the polynucleotide comprises any one of SEQ ID NO:96-119.

17. A composition comprising a polynucleotide that binds a nucleic acid-binding polypeptide selected from TDP-43, FUS, HNRNPA1, HNRNPA2/B1, EWSR1, TIA-1, TTP, TAF15, MATR3, ATXN2, OPTN, Tau, HTT, and biologically active fragments and variants thereof, wherein the nucleic acid-binding polypeptide can aggregate in cells, and is associated with a neurodegenerative disease.

18. The composition of claim 17, wherein the polynucleotide comprises 100 nucleotides or less.

19. A pharmaceutical composition comprising the polynucleotide of claim 17, and a pharmaceutically acceptable excipient.

20. A The composition of claim 17, wherein the polynucleotide comprises any one of SEQ ID NO:10-14, 33-56, 61-84, 99-101, and 106-119.

21. The composition of claim 20, wherein the polynucleotide comprises any one of SEQ ID NO:10-14, 33-56, and 61-84.

22. The composition of claim 20, wherein the polynucleotide comprises any one of SEQ ID NO:10, SEQ ID NO:11, SEQ ID NO:12, SEQ ID NO:13, and SEQ ID NO:14.

23. The composition of claim 20, wherein the polynucleotide comprises any one of SEQ ID NO: 99-101, and 106-119.

24. The composition of claim 17, wherein the polynucleotide comprises
SEQ ID NO:85, SEQ ID NO:62, and SEQ ID NO:86, in that order;
SEQ ID NO:87, SEQ ID NO:63, and SEQ ID NO:88, in that order;
SEQ ID NO:92, SEQ ID NO:64, and SEQ ID NO:92, in that order;
SEQ ID NO:92, SEQ ID NO:65, and SEQ ID NO:92, in that order;
SEQ ID NO:92, SEQ ID NO:66, and SEQ ID NO:92, in that order;
SEQ ID NO:92, SEQ ID NO:67, and SEQ ID NO:92, in that order;
SEQ ID NO:92, SEQ ID NO:68, and SEQ ID NO:92, in that order;
SEQ ID NO: 92, SEQ ID NO:69, and SEQ ID NO:92, in that order;
SEQ ID NO:92, SEQ ID NO:70, and SEQ ID NO:92, in that order;
SEQ ID NO:92, SEQ ID NO:71, and SEQ ID NO:92, in that order;
SEQ ID NO:92, SEQ ID NO:72, and SEQ ID NO:92, in that order;
SEQ ID NO:92, SEQ ID NO:73, and SEQ ID NO:92, in that order;
SEQ ID NO:92, UGUGUGUG, and SEQ ID NO:92, in that order;
SEQ ID NO:92, UGUGUG, and SEQ ID NO:92, in that order;
SEQ ID NO:89, SEQ ID NO:74, and SEQ ID NO:89, in that order;
SEQ ID NO:90, SEQ ID NO:74, and SEQ ID NO:90, in that order;
SEQ ID NO:91, SEQ ID NO:74, and SEQ ID NO:91, in that order;
SEQ ID NO:92, SEQ ID NO:74, and SEQ ID NO:92, in that order;
SEQ ID NO:93, SEQ ID NO:74, and SEQ ID NO:93, in that order;
SEQ ID NO:94, SEQ ID NO:74, and SEQ ID NO:94, in that order;
SEQ ID NO:95, SEQ ID NO:74, and SEQ ID NO:95, in that order;
CGCGCGCG, SEQ ID NO:74, and CGCGCGCG, in that order;
CGCGCG, SEQ ID NO:74, and CGCGCG, in that order;
CGCG, SEQ ID NO:74, and CGCG, in that order;
CG, SEQ ID NO:74, and CG, in that order;
SEQ ID NO:92, SEQ ID NO:75, and SEQ ID NO:92, in that order;
SEQ ID NO:92, SEQ ID NO:76, and SEQ ID NO:92, in that order;
SEQ ID NO: 92, SEQ ID NO:77, and SEQ ID NO:92, in that order;
SEQ ID NO:92, SEQ ID NO:78, and SEQ ID NO:92, in that order;
SEQ ID NO:92, SEQ ID NO:79, and SEQ ID NO:92, in that order;
SEQ ID NO:92, SEQ ID NO:80, and SEQ ID NO:92, in that order;
SEQ ID NO:92, SEQ ID NO:81, and SEQ ID NO:92, in that order;
SEQ ID NO:92, SEQ ID NO:82, and SEQ ID NO:92, in that order;
SEQ ID NO:92, SEQ ID NO:83, and SEQ ID NO:92, in that order;
SEQ ID NO:92, SEQ ID NO:84, and SEQ ID NO:92, in that order;
SEQ ID NO:92, TGTGTGTG, and SEQ ID NO:92, in that order; or
SEQ ID NO:92, TGTGTG, and SEQ ID NO:92, in that order.
